(19) United States
(12) Patent Application Publication
(10) Pub. No.: US 2024/0298968 A1
(43) Pub. Date: Sep. 12, 2024

(54) Smart Bra with Light Transmission for Optical Detection of Abnormal Breast Tissue

(71) Applicant: Robert A. Connor, Wyoming, MN (US)

(72) Inventor: Robert A. Connor, Wyoming, MN (US)

(73) Assignee: Holovisions LLC, Ham Lake, MN (US)

(21) Appl. No.: 18/666,352

(22) Filed: May 16, 2024

Related U.S. Application Data
(63) Continuation-in-part of application No. 18/237,231, filed on Aug. 23, 2023.
Application No. 18/237,231 is a continuation-in-part of application No. 18/096,748, filed on Jan. 13, 2023.
Application No. 18/096,748 is a continuation-in-part of application No. 17/897,182, filed on Aug. 28, 2022, now Pat. No. 11,950,881.
Application No. 17/897,182 is a continuation-in-part of application No. 16/933,138, filed on Jul. 20, 2020, now abandoned.
(60) Provisional application No. 63/648,156, filed on May 15, 2024.
(60) Provisional application No. 62/879,485, filed on Jul. 28, 2019.

(51) Int. Cl. A61B 5/00; A41C 3/00

(52) U.S. Cl. CPC A61B 5/6804; A41C 3/0028; A61B 5/0091

(57) ABSTRACT

A smart bra enables optical detection of abnormal breast tissue. A cup on the bra has an inelastic portion which is closer to a person's chest wall and an elastic portion which is farther from the chest wall. Light from light emitters on one side of the cup is transmitted through a breast to light receivers on the other side of the cup. Changes in this light caused by interaction with tissue are analyzed to detect abnormal breast tissue. Expandable chambers in the cup gently compress the breast to improve optical scanning.

### CROSS-REFERENCE TO RELATED APPLICATIONS

This application claims the priority benefit of U.S. provisional application No. 63/648,156 filed on 2024 May 15. This application is a continuation-in-part of U.S. application Ser. No. 18/237,231 filed on 2023 Aug. 23. U.S. application Ser. No. 18/237,231 was a continuation-in-part of U.S. application Ser. No. 18/096,748 filed on 2023 Jan. 13. U.S. application Ser. No. 18/096,748 was a continuation-in-part of U.S. application Ser. No. 17/897,182 filed on 2022 Aug. 28 which issued as U.S. Pat. No. 11,950,881 on 2024 Apr. 9. U.S. application Ser. No. 17/897,182 was a continuation-in-part of U.S. application Ser. No. 16/933,138 filed on 2020 Jul. 20. U.S. application Ser. No. 16/933,138 claimed the priority benefit of U.S. provisional application No. 62/879,485 filed on 2019 Jul. 28. The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to wearable devices for medical imaging and diagnosis.

### Introduction

Early detection of breast cancer is vital. Current methods for breast cancer detection include x-ray mammography, MRI, and ultrasound. However, these methods have limitations. X-ray mammography causes exposure to ionizing radiation and is not very mobile, MRI equipment is expensive and not very mobile, and ultrasound can be labor-intensive and have low sensitivity. Optical imaging and spectroscopic analysis is another method for breast cancer detection which can address these limitations.

Optical imaging and spectroscopic analysis of breast tissue takes advantage of differences in light absorption and scattering by normal tissue vs. abnormal tissue. There are several biomarkers which are present in different concentrations in abnormal tissue. These biomarkers include deoxygenated hemoglobin, oxygenated hemoglobin, lipids, collagen, oxygen, and water. When near-infrared light is transmitted through and/or reflected from breast tissue, changes in light transmission caused by these biomarkers can be used to detect and image abnormal breast tissue.

With continuous wave (CW) optical analysis methods, light emitters direct light with constant intensity into and/or onto breast tissue. With frequency domain (FD) optical analysis methods, light emitters direct modulated light into and/or onto breast tissue. With time domain (TD) optical analysis methods, light emitters direct pulses of light into and/or onto breast tissue.

Variations on optical imaging and spectroscopic analysis methods in the prior art include: Diffuse Optical Imaging (DOI), Diffuse Optical Spectroscopic Imaging (DOSI), Diffuse Optical Spectroscopy (DOS), Diffuse Optical Tomography (DOT), Frequency-Domain Photon Migration (FDPM), Functional Near-Infrared Spectroscopy (fNIRS), Near-Infrared Spectroscopy (NIRS), Raman spectroscopy, Reflectance Diffuse Optical Tomography (RDOT), Transillumination Imaging (TI), and/or Transmittance Diffuse Optical Tomography (TDOT).

Although optical methods for detection of breast cancer are promising, there some limitations with devices and methods in the prior art. One challenge is the difficulty of using optical methods to scan greater tissue depth because light is increasingly scattered through larger spans of tissue. There remains a need for innovative optical devices and methods to detect breast cancer which solve these challenges.

### Review of the Relevant Art

In the patent literature, U.S. patent application 20050043596 (Chance, Feb. 24, 2005, “Optical Examination Device, System and Method”) discloses a brush-form optical coupler with freely extending fiber end portions, sized and positioned to make optical contact with a subject, examination, and monitoring systems utilizing one or more of such couplers. U.S. patent application 20060058683 (Chance, Mar. 16, 2006, “Optical Examination of Biological Tissue Using Non-Contact Irradiation and Detection”) and U.S. Pat. No. 7,904,139 (Chance, Mar. 8, 2011, “Optical Examination of Biological Tissue Using Non-Contact Irradiation and Detection”) disclose an optical system for examination of biological tissue which includes a light source, a light detector, optics and electronics. Sometimes inventions are the result of serendipitous insights; in this case, optical scanning of biological tissue may actually have been invented by chance.

U.S. Pat. No. 6,081,322 (Barbour, Jun. 27, 2000, “NIR Clinical Opti-Scan System”) and RE38800 (Barbour, Sep. 20, 2005, “NIR Clinical Opti-Scan System”) disclose three-dimensional optical imaging techniques for the detection and three-dimensional imaging of absorbing and/or scattering structures in complex random media, such as human body tissue, by detecting scattered light. U.S. patent application 20150182121 (Barbour, Jul. 2, 2015, “Low-Cost Screening System for Breast Cancer Detection”) discloses a portable and wearable tumor detector including a brassier and devices for optical tomography. U.S. patent application publication 20150119665 (Barbour et al., Apr. 30, 2015, “Self-Referencing Optical Measurement for Breast Cancer Detection”) and U.S. Pat. No. 9,724,489 (Barbour et al., Aug. 8, 2017. “Self-Referencing Optical Measurement for Breast Cancer Detection”) disclose obtaining optical data from a pair of breasts, employing a simultaneous bilateral referencing protocol, and employing a self-referencing data analysis method.

U.S. patent applications 20100292569 (Hielscher et al., Nov. 18, 2010, “Systems and Methods for Dynamic Imaging of Tissue Using Digital Optical Tomography”) and 20150223697 (Hielscher et al., Aug. 13, 2015, “Systems and Methods for Dynamic Imaging of Tissue Using Digital Optical Tomography”) disclose methods for imaging tissue using diffuse optical tomography including directing a amplitude modulated optical signals from optical signal sources. U.S. patent application 20140330116 (Hielscher et al., Nov. 6, 2014, “Systems and Methods for Simultaneous Multi-Directional Imaging for Capturing Tomographic Data”) discloses devices, systems, and method for tomographic imaging in which light transmitted and backscattered surface light is imaged by an optical system that minimizes reflection back to the target object. U.S. patent applications 20130289394 (Hielscher et al., Oct. 31, 2013, “Dynamic Optical Tomographic Imaging Devices Methods and Systems”), 20170027480 (Hielscher et al., Feb. 2, 2017, “Dynamic Optical Tomographic Imaging Devices Methods and Systems”), and 20190282134 (Hielscher et al., Sep. 19, 2019, “Dynamic Optical Tomographic Imaging Devices Methods and Systems”), and U.S. Pat. No. 10,178,967 (Hielscher et al., Jan. 15, 2019, “Dynamic Optical Tomographic Imaging Devices Methods and Systems”) disclose an optical tomographic systems for acquiring and displaying dynamic data representing changes in a target tissue sample to external provocation. U.S. patent applications 20130338496 (Hielscher et al., Dec. 19, 2013, “Medical Imaging Devices, Methods, and Systems”) and 20140088415 (Hielscher et al., Mar. 27, 2014, “Medical Imaging Devices, Methods, and Systems”) disclose devices, methods, and systems for generating optical tomographic data including volumetric and surface geometric data.

U.S. patent application publication 20140236003 (Hielscher et al., Aug. 21, 2014, “Interfacing Systems, Devices, and Methods for Optical Imaging”) discloses an imaging interface with a plurality of concentric rings for diffuse optical tomography of breast tissue. U.S. patent applications 20140243681 (Hielscher et al., Aug. 28, 2014, “Compact Optical Imaging Devices, Systems, and Methods”) and 20190239751 (Hielscher et al., Aug. 8, 2019, “Compact Optical Imaging Devices, Systems, and Methods”), and U.S. Pat. No. 10,111,594 (Hielscher et al., Oct. 30, 2018, “Compact Optical Imaging Devices, Systems, and Methods”) disclose a handheld optical imaging system with a plurality of detectors. U.S. patent application 20150286785 (Hielscher et al., Oct. 8, 2015, “Systems, Methods, and Devices for Image Reconstruction Using Combined PDE-Constrained and Simplified Spherical Harmonics Algorithm”) and U.S. Pat. No. 9,495,516 (Hielscher et al., Nov. 15, 2016, “Systems, Methods, and Devices for Image Reconstruction Using Combined PDE-Constrained and Simplified Spherical Harmonics Algorithm”) disclose systems, methods, and devices for image reconstruction using combined PDE-constrained and simplified spherical harmonics (SPN) algorithms. U.S. Pat. No. 10,376,150 (Hielscher et al., Aug. 13, 2019, “Interfacing Systems, Devices, and Methods for Optical Imaging”) discloses an imaging interface for diffuse optical tomography of breast with a plurality of concentric rings.

U.S. patent application publication 20140236021 (Islam, Aug. 21, 2014, “Near-Infrared Super-Continuum Lasers for Early Detection of Breast and Other Cancers”) and U.S. Pat. No. 9,993,159 (Islam, Jun. 12, 2018, “Near-Infrared Super-Continuum Lasers for Early Detection of Breast and Other Cancers”) disclose a system and method using near-infrared or short-wave infrared light sources for early detection and monitoring of breast cancer. U.S. patent application publication 20180289264 (Islam, Oct. 11, 2018, “High Signal-to-Noise Ratio Light Spectroscopy of Tissue”) discloses a diagnostic system which delivers an optical beam to a nonlinear element that broadens a spectrum of the first optical beam to at least 10 nanometers through a nonlinear effect in the nonlinear element. U.S. patent application 20210038083 (Islam, Feb. 11, 2021, “Multi-Wavelength Wearable Device for Non-Invasive Blood Measurements in Tissue”) discloses a system for measuring one or more physiological parameters with a wearable device that includes a light source comprising a driver and semiconductor sources that generate an output optical light.

U.S. patent application publication 20090005692 (Intes et al., Jan. 1, 2009, “Optical Imaging Method for Tissue Characterization”) and U.S. Pat. No. 8,565,862 (Intes et al., Oct. 22, 2013, “Optical Imaging Method for Tissue Characterization”) disclose a method for detecting and characterizing abnormalities within biological tissue by characterizing optical properties of the tissue. U.S. patent application publication 20180070891 (Jepsen, Mar. 15, 2018, “Imaging With Infrared Imaging Signals”) discloses using an infrared imaging signal to image tissue. U.S. patent application publication 20180335753 (Jepsen et al., Nov. 22, 2018, “Co-Located Imaging and Display Pixel”) discloses an optical transformation engine coupled between an image pixel and a display pixel. U.S. patent application publication 20190072897 (Jepsen et al., Mar. 7, 2019, “Applications of Diffuse Medium Imaging”) discloses methods and an apparatus for imaging translucent materials.

U.S. Pat. No. 9,314,218 (Stearns et al., Apr. 19, 2016, “Integrated Microtomography and Optical Imaging Systems”) and 10130318 (Stearns et al., Nov. 20, 2018, “Integrated Microtomography and Optical Imaging Systems”) disclose an integrated microtomography and optical imaging system with a rotating table that supports an imaging object, an optical stage, and separate optical and microtomography imaging systems. U.S. Pat. No. 9,770,220 (Stearns et al., Sep. 26, 2017, “Integrated Microtomography and Optical Imaging Systems”) discloses a rotating table that supports an imaging object, an optical stage, and separate optical and microtomography imaging systems. U.S. patent application 20170209083 (Zarandi et al., 2017, “Hand-Held Optical Scanner for Real-Time Imaging of Body Composition and Metabolism”) and U.S. Pat. No. 10,653,346 (Zarandi et al., May 19, 2020, “Hand-Held Optical Scanner for Real-Time Imaging of Body Composition and Metabolism”) disclose a handheld system for diffuse optical spectroscopic imaging of human tissue.

U.S. patent application 20060173352 (Lilge et al., 2006, “Optical Transillumination and Reflectance Spectroscopy to Quantify Disease Risk”) discloses a method of illuminating tissue of a mammal with light having wavelengths covering a pre-selected spectral range, detecting light transmitted through, or reflected from, the volume of selected tissue, and obtaining a spectrum of the detected light. U.S. patent application 20200116630 (Zhu, 2020, “Compact Guided Diffuse Optical Tomography System for Imaging a Lesion Region”) discloses a compact diffuse optical tomography system with laser diodes and a laser diode driver board. U.S. Pat. No. 5,876,339 (Lemire, Mar. 2, 1999, “Apparatus for Optical Breast Imaging”) discloses an optical breast imager with an adjustable volume which encloses a patient's breast.

U.S. Pat. No. 5,999,836 (Nelson et al., Dec. 7, 1999, “Enhanced High Resolution Breast Imaging Device and Method Utilizing Non-Ionizing Radiation of Narrow Spectral Bandwidth”) and 6345194 (Nelson et al., Feb. 5, 2002, “Enhanced High Resolution Breast Imaging Device and Method Utilizing Non-Ionizing Radiation of Narrow Spectral Bandwidth”) disclose breast imaging using collimated non-ionizing radiation in the near ultraviolet, visible, infrared, and microwave regions. U.S. Pat. No. 6,240,309 (Yamashita et al., May 29, 2001, “Optical Measurement Instrument for Living Body”), 6640133 (Yamashita et al., Oct. 28, 2003, “Optical Measurement Instrument for Living Body”), and 7142906 (Yamashita et al., Nov. 28, 2006, “Optical Measurement Instrument for Living Body”) disclose an optical measurement instrument which applies visible-infrared light to several positions on a patient.

U.S. patent application 20020045833 (Wake et al., Apr. 18, 2002, “Medical Optical Imaging Scanner Using Multiple Wavelength Simultaneous Data Acquisition for Breast Imaging”) discloses a scanner for a medical optical imaging device with an illumination source which directs emitted light into a breast positioned below a support surface. U.S. Pat. No. 6,571,116 (Wake et al., May 27, 2003, “Medical Optical Imaging Scanner Using Multiple Wavelength Simultaneous Data Acquisition for Breast Imaging”) and 6738658 (Wake et al., May 18, 2004, “Medical Optical Imaging Scanner Using Multiple Wavelength Simultaneous Data Acquisition for Breast Imaging”) disclose a medical optical imaging device with an illumination source that directs emitted light into a breast positioned below a support surface.

U.S. patent application publication 20040092826 (Corbeil et al., May 13, 2004, “Method and Apparatus for Optical Imaging”) and U.S. Pat. No. 7,809,422 (Corbeil et al., Oct. 5, 2010, “Method and Apparatus for Optical Imaging”) disclose a platform with a cavity into which one of the person's breasts is suspended for optical imaging. U.S. patent application publication 20070287897 (Faris, Dec. 13, 2007, “Optical Vascular Function Imaging System and Method for Detection and Diagnosis of Cancerous Tumors”) discloses an in-vivo optical imaging system and method of identifying unusual vasculature associated with tumors. U.S. Pat. No. 8,027,711 (Jones et al., Sep. 27, 2011, “Laser Imaging Apparatus with Variable Patient Positioning”) discloses a tabletop to support a patient in front-down position and an opening to permit a breast of the patient to be vertically pendant below the tabletop.

U.S. Pat. No. 8,224,426 (Lilge et al., Jul. 17, 2012, “Optical Transillumination and Reflectance Spectroscopy to Quantify Disease Risk”) discloses spectroscopic tissue volume measurements with non-ionizing radiation to detect pre-disease transformations in tissue. U.S. patent application publication 20160066811 (Mohamadi, Mar. 10, 2016, “Handheld and Portable Scanners for Millimeter Wave Mammography and Instant Mammography Imaging”) discloses an array of ultra-wide band radio frequency sensors for breast imaging. U.S. Pat. No. 9,513,276 (Tearney et al., Dec. 6, 2016, “Method and Apparatus for Optical Imaging via Spectral Encoding”) disclose a method, apparatus and arrangement for obtaining information associated with a sample such as a portion of an anatomical structure. U.S. patent application publication 20170007187 (Breneisen et al., Jan. 12, 2017, “Cancer Detector Using Deep Optical Scanning”) discloses Deep Optical Scanning (DEOS) for the detection of breast cancer and the determination of response to therapy.

U.S. Pat. No. 9,597,046 (Goossen et al., Mar. 21, 2017, “Method and Device for Imaging Soft Body Tissue Using X-Ray Projection and Optical Tomography”) discloses breast imaging using X-ray projection techniques and optical tomography techniques. U.S. patent application 20170105625 (Eum, Apr. 20, 2017, “Diagnostic Device of Optics Type for Breast”) discloses an optical breast diagnostic apparatus with a hemispherical cover. U.S. patent Ser. No. 10/200,655 (Kim et al., Feb. 5, 2019, “Tomographic Imaging Methods, Devices, and Systems”) discloses a multispectral bioluminescence optical tomography algorithm makes use of a partial differential equation (PDE) constrained approach. U.S. Pat. No. 10,215,636 (Fujii et al., Feb. 26, 2019, “Imaging Device Provided With Light Source That Emits Pulsed Light and Image Sensor”) discloses an imaging device with a light source that emits pulsed light at different wavelengths. U.S. Pat. No. 10,506,181 (Delgado et al., Dec. 10, 2019, “Device for Optical Imaging”) discloses the capture of an infrared image.

Turning to the non-patent literature, Ahmed et al., (2021), “Differential Optical Absorption Spectroscopy-Based Refractive Index Sensor for Cancer Cell Detection,” Optical Review, 28, 134-143, discloses a spectroscopic optical sensor for cancerous cell detection in various parts of the human body. Altoe et al., (2019), “Diffuse Optical Tomography of the Breast: A Potential Modifiable Biomarker of Breast Cancer Risk with Neoadjuvant Chemotherapy,” Biomedical Optics Express, Aug. 1, 2019, 10(8), 4305-4315, studied whether a diffuse optical tomography breast imaging system (DOTBIS) can provide a comparable optical-based image index of mammographic breast density. Altoe et al., (2021), “Changes in Diffuse Optical Tomography Images During Early Stages of Neoadjuvant Chemotherapy Correlate with Tumor Response in Different Breast Cancer Subtypes”, Clinical Cancer Research, Apr. 1, 2021, 27(7), 1949-1957, studied changes in optically derived parameters acquired with a diffuse optical tomography breast imaging system (DOTBIS) in the tumor volume of patients with breast carcinoma receiving neoadjuvant chemotherapy (NAC).

Altoe et al., (2021), “Effects of Neoadjuvant Chemotherapy on the Contralateral Non-Tumor-Bearing Breast Assessed by Diffuse Optical Tomography,” Breast Cancer Research, 2021, 23, 16, studied whether changes in optically derived parameters acquired with a diffuse optical tomography breast imager system (DOTBIS) in the contralateral non-tumor-bearing breast in patients administered neoadjuvant chemotherapy (NAC) for breast cancer are associated with pathologic complete response (pCR). Anabestani et al. (2022), “Advances in Flexible Organic Photodetectors: Materials and Applications,” Nanomaterials, 2022, 12(21), 3775, discusses recent advances in flexible organic photodetectors, including their applications in health-monitoring, X-ray detection, and imaging. Anderson et al., (2017), “Optical Mammography in Patients with Breast Cancer Undergoing Neoadjuvant Chemotherapy: Individual Clinical Response Index,” Academic Radiology, October 2017, 24(10), 1240-1255, discloses an optical mammography study to develop quantitative measures of pathologic response to neoadjuvant chemotherapy (NAC) in patients with breast cancer.

Angelo et al., (2018), “Review of Structured Light in Diffuse Optical Imaging,” Journal of Biomedical Optics, Sep. 14, 2018, 24(7), 071602, discloses diffuse optical imaging probes in living tissue enabling structural, functional, metabolic, and molecular imaging. Applegate et al., (2018), “Multi-Distance Diffuse Optical Spectroscopy with a Single Optode via Hypotrochoidal Scanning,” Optics Letters, 2018, 43, 747-750, studied a new method of frequency-domain diffuse optical spectroscopy (FD-DOS) to rapidly acquire a wide range of source-detector (SD) separations by mechanically scanning a single SD pair. Applegate et al. (2020), “Recent Advances in High Speed Diffuse Optical Imaging in Biomedicine,” APL Photonics, 2020, 5(4), 040802, 21, reviews recent advances in acquisition and processing speed for several Diffuse Optical Imaging modalities.

Chae et al., (2020), “Development of Digital Breast Tomosynthesis and Diffuse Optical Tomography Fusion Imaging for Breast Cancer Detection,” Scientific Reports, 10. 13127 (2020), studied a new digital breast tomosynthesis (DBT)/DOT fusion imaging technique for breast cancer detection. Chitnis et al. (2016), “Towards a Wearable Near Infrared Spectroscopic Probe for Monitoring Concentrations of Multiple Chromophores in Biological Tissue In Vivo,” Review of Scientific Instruments, June 2016, 87(6), 065112, discloses a wearable multi-wavelength technology for functional near-infrared spectroscopy with an 8-wavelength light emitting diode (LED) source. Cinquino et al. (2021), “Light-Emitting Textiles: Device Architectures, Working Principles, and Applications,” Micromachines, (Special Issue Emerging and Disruptive Next-Generation Technologies for POC: Sensors, Chemistry and Microfluidics for Diagnostics), 2021, 12(6), 652, discusses applications of light-emitting fabrics, including Organic LEDs.

Cochran et al., (2019), “Hybrid Time-Domain and Continuous-Wave Diffuse Optical Tomography Instrument with Concurrent, Clinical Magnetic Resonance Imaging for Breast Cancer Imaging.” Journal of Biomedical Optics, January 2019, 24(5), 1-11, discusses diffuse optical tomography (DOT) for three-dimensional (3-D) maps of tissue optical and physiological properties in human tissue. Costa et al. (2019), “Flexible Sensors: From Materials to Applications,” Technologies (Special Issue, Reviews and Advances in Internet of Things Technologies), 2019, 7(2), 35, reviews the current state of flexible sensor technologies and the impact of material developments on this field. Durduran et al., (2010, 2010), “Diffuse Optics for Tissue Monitoring and Tomography.” Reports on Progress in Physics, 2010, 73(7), 076701, discloses using near-infrared or diffuse optical spectroscopy to measure tissue hemodynamics.

Fakayode et al., (2020), “Molecular (Raman, NIR, and FTIR) Spectroscopy and Multivariate Analysis in Consumable Products Analysis.” Applied Spectroscopy, reviews, 55:8, 647-723, reviews the use of Raman, near-infrared (NIR), and Fourier-transform infrared (FTIR) spectrometers to evaluate consumable products such as food. Fantini et al., (2001), “Optical Spectroscopy and Imaging of Tissues,” NSF Award #0093840, Jun. 1, 2001, studied development of new improved methods and instrumentation for biomedical applications of near-infrared spectroscopy and imaging. Fantini (2005), “Optical Spectroscopy and Imaging of Tissues”. NSF Award, 2005 (abstract only viewed), researched techniques for optical spectroscopy and imaging of biological tissues. Fantini et al., (2012), “Near-Infrared Optical Mammography for Breast Cancer Detection with Intrinsic Contrast,” Annals of Biomedical Engineering, February 2012, 40(2), 398-407, reviews optical methods to detect breast cancer on the basis of increased opacity.

Farmani et al., (2020), “Optical Nanosensors for Cancer and Virus Detections,” Micro and Nano Technologies, Nanosensors for Smart Cities, Chapter 25, Han et al. editors, Elsevier, 2020, 419-432, ISBN 9780128198704, discusses photonic crystal (PhC)-based optical nanosensors. Feng et al. (2021), “MRI Guided Wearable Near Infrared Spectral Tomography: Simulation Study,” Proceedings SPIE, 11639, Optical Tomography and Spectroscopy of Tissue XIV, 116390D, Mar. 5, 2021, discloses a new low-cost imaging system for MRI-guided Near-Infrared Spectral Tomography (MRI-NIRST) for breast cancer detection. Flexman et al., (2008), “The Design and Characterization of a Digital Optical Breast Cancer Imaging System,” 30th Annual International Conference of the IEEE Engineering in Medicine and Biology Society, 2008, 3735-3738, discusses how optical imaging has the potential to play a major role in breast cancer screening and diagnosis due to its ability to image cancer characteristics such as angiogenesis and hypoxia.

Ghijsen et al., (2018), “Quantitative Real-Time Optical Imaging of the Tissue Metabolic Rate of Oxygen Consumption,” Journal of Biomedical Optics, Mar. 24, 2018, 23(3), 036013, discloses a noncontact method for quantitatively mapping tMRO2 over a wide, scalable field of view. Grosenick et al., (2016), “Review of Optical Breast Imaging and Spectroscopy.” Journal of Biomedical Optics, September 2016, 21(9), 091311, reviews the monitoring neoadjuvant chemotherapy and breast cancer risk assessment via optical breast imaging and spectroscopy. Gunther et al., (2018), “Dynamic Diffuse Optical Tomography for Monitoring Neoadjuvant Chemotherapy in Patients with Breast Cancer,” Radiology, June 2018; 287(3): 778-786, identifies dynamic optical imaging features associated with pathologic response in patients with breast cancer during neoadjuvant chemotherapy.

Hoi et al., (2018), “Non-Contact Dynamic Diffuse Optical Tomography Imaging System for Evaluating Lower Extremity Vasculature,” Biomedical Optics Express, 2018, 9, 5597-5614, discloses a multi-view non-contact dynamic diffuse optical tomographic imaging system for the clinical evaluation of vasculature in the lower extremities. Imamura et al., (2018), “In Vivo Optical Imaging of Cancer Cell Function and Tumor Microenvironment,” Cancer Science, 2018, 109, 912-918, discusses in vivo optical imaging using fluorescence and bioluminescence. Intes et al. (2004), “Time-Domain Optical Mammography Softscan: Initial Results On Detection and Characterization of Breast Tumors,” Proceedings SPIE 5578, Photonics North 2004: Photonic Applications in Astronomy, Biomedicine, Imaging, Materials Processing, and Education, Dec. 9, 2004 presents initial results obtained using a breast-imaging system developed by Advanced Research Technologies comprising a 4-wavelength time-resolved scanning system.

Jeong et al., (2020), “Emerging Advanced Metasurfaces: Alternatives to Conventional Bulk Optical Devices,” Microelectronic Engineering, 2020, Vol. 220, 111146, ISSN 0167-9317, discusses the use of optical metasurfaces as color filters, metalenses, beam generators or splitters, and meta-holograms. Jiang et al. (2021), “MRI-Guide Near Infrared Spectroscopic Tomographic Imaging System with Wearable Optical Breast Interface for Breast Imaging.” Proceedings SPIE, 11639, Optical Tomography and Spectroscopy of Tissue XIV, 116391J, Mar. 5, 2021, discloses a new photo-detector (PD) and source fiber based wearable MRI-guide near infrared spectroscopic tomographic imaging (MRg-NIRST) system. Joshi et al., (2018), “Targeted Optical Imaging Agents in Cancer: Focus on Clinical Applications,” Contrast Media and Molecular Imaging. Aug. 27, 2018, discusses molecular imaging for in vivo visualization of cancer over time based on biological mechanisms of disease activity.

Jung et al. (2015), “Non-Contact Deep Tissue Imaging using a Hand-Held Near-infrared Optical Scanner,” Journal of Medical Diagnostic Methods, Mar. 24, 2015, 4(2), 1-10, discloses fiber-free non-contact near-infrared (NIR) imaging devices using wide-field detectors. Khan (2013), “Image Reconstruction in Diffuse Optical Tomography With Sparsity Constraints”, NSF Award, 2013 (abstract only viewed), researched the use of sparsity-constrained regularization for solving the diffuse optical tomography inverse problem. Kim et al., (2016), “US-Localized Diffuse Optical Tomography in Breast Cancer: Comparison with Pharmacokinetic Parameters of DCE-MRI and With Pathologic Biomarkers,” BMC Cancer, Feb. 1, 2016, 16:50, discloses correlating parameters of ultrasonography-guided diffuse optical tomography with the pharmacokinetic features of dynamic contrast-enhanced MRI and pathologic markers of breast cancer. Koetse et al., (2007), “Optical Sensor Array Platform Based on Polymer Electronic Devices,” Proceedings SPIE 6739, Electro-Optical Remote Sensing, Detection, and Photonic Technologies and Their Applications, 67391D, Nov. 7, 2007, discusses devices based on polymer semiconductors fabricated with thin film technology.

Koomson, 2019), “A Noninvasive Biological Research Tool for Measurement of Tissue and Cerebral Oxygenation,” NSF Award #1919038, Jul. 15, 2019, (abstract only viewed) investigates compact wearable devices with advanced NIRS capability. Krishnamurthy, 2018), “Using Near-Infrared Spectroscopy to Study Static and Dynamic Hemoglobin Contrast Associated with Breast Cancer,” Tufts University, Dissertation, 2018, discloses an instrument for diffuse optical mammography with parallel plate geometry. Leo et al. (2017), “Optical Imaging of the Breast: Basic Principles and Clinical Applications,” American Journal of Roentgenology, 2017, 209:1, 230-238, discloses summarizes the physical principles, technology features, and first clinical applications of optical imaging techniques to the breast. Li et al. (2018), “Sensitive and Wearable Optical Microfiber Sensor for Human Health Monitoring,” Advanced Materials Technologies, 2018, 3, 1800296, discloses a sensor with a hybrid plasmonic microfiber knot resonator embedded in a polydimethylsiloxane membrane.

Liu et al., (2018), “Diffuse Optical Spectroscopy for Monitoring the Responses of Patients with Breast Cancer to Neoadjuvant Chemotherapy: A Meta-Analysis,” Medicine, 2018, 97(41), 12683, investigated the potential of diffuse optical spectroscopy (DOT) for monitoring the responses of patients with breast cancer to neoadjuvant chemotherapy (NAC). Liu et al., (2020), “Recent Progress in Flexible Wearable Sensors for Vital Sign Monitoring.” Sensors, 2020, 20(14), 4009, discusses the development of flexible electronic materials, as well as the wide development and application of smartphones, the cloud, and wireless systems, flexible wearable sensor technology. Liu et al., (2021), “Simultaneous Measurements of Tissue Blood Flow and Oxygenation Using a Wearable Fiber-Free Optical Sensor,” Journal of Biomedical Optics, Jan. 29, 2021, 26(1), 012705, discusses a wearable dual-wavelength diffuse speckle contrast flow oximetry (DSCFO) device for simultaneous measurements of blood flow and oxygenation variation in deep tissues.

Lutzweiler et al., (2013), “Optoacoustic Imaging and Tomography: Reconstruction Approaches and Outstanding Challenges in Image Performance and Quantification,” Sensors, 2013, 13(3), 7345-7384, reviews optoacoustic imaging from image reconstruction and quantification perspectives. Ma et al. (2020b), “Fiber-Free Parallel-Plane Continuous Wave Breast Diffuse Optical Tomography System.” SPIE 11229, Advanced Biomedical and Clinical Diagnostic and Surgical Guidance Systems XVIII, Proceedings, 112290L, Feb. 21, 2020 discusses near infrared diffuse optical tomography (DOT) for detecting breast cancer. Mabou et al., (2018), “Breast Cancer Detection Using Infrared Thermal Imaging and a Deep Learning Model,” Sensors, 2018, 18(9), 2799, discloses the use of infrared digital imaging for breast cancer detection based on thermal comparison between a healthy breast and a breast with cancer.

Moreno et al. (2019), “Evaluation on Phantoms of the Feasibility of a Smart Bra to Detect Breast Cancer in Young Adults,” Sensors, 2019, 19(24), 5491, discloses the use of breast tissue phantoms to investigate the feasibility of quantifying breast density and detecting breast cancer tumors using a smart bra. Nguyen et al., (2020), “Preliminary Development of Optical Computed Tomography (Optical CT) Scanner Using Transillumination Imaging NAD,” Conference: International Symposium on Applied Science 2019, Hochiminh City, Vietnam, May 14, 2020, discusses the use of near-infrared transillumination imaging for biomedical applications such as human biometrics and animal experiments. Pan et al., (2020), “A Multifunctional Skin-Like Wearable Optical Sensor Based on an Optical Micro-/Nanofibre,” Nanoscale, 2020, Issue 33, discusses multifunctional skin-like sensors for next-generation healthcare, robotics, and bioelectronics.

Park et al., (2013), “Multispectral Imaging Using Polydimethylsiloxane (PDMS) Embedded Vertical Silicon Nanowires,” OSA Technical Digest (online) (Optical Society of America, 2013), paper CTu3O.1, reports on the demonstration of a compact multispectral imaging system that uses vertical silicon nanowires for a filter array. Park et al., (2015), “Vertically Stacked Photodetector Devices Containing Silicon Nanowires with Engineered Absorption Spectra.” ACS Photonics, Mar. 16, 2015, 2(4), 544-549, discloses a vertically stacked photodetector device containing silicon nanowire photodetectors formed above a silicon substrate that also contains a photodetector. Perumal et al., (2019), “Near Infra-Red Polymeric Nanoparticle Based Optical Imaging in Cancer Diagnosis,” Journal of Photochemistry and Photobiology, Biology, 2019, Vol. 199, 111630, ISSN 1011-1344, reviews the recent progress in NIRF polymeric nanoparticles used for optical imaging particularly on cancer diagnosis.

Pinti et al. (2018), “A Review on the Use of Wearable Functional Near-Infrared Spectroscopy in Naturalistic Environments,” Japanese Psychology Research, October 2018, 60(4), 347-373, reviews the use of wearable fNIRS in naturalistic settings in the field of cognitive neuroscience. Qiu (2018), “Implantable Ultra-low Power VO2 MEMS Scanner Based Surface-Enhanced Raman Spectroscope for Wide-field Tumor Imaging in Free Moving Small Animals”, NSF Award, 2018 (abstract only viewed), discloses tumor-targeting surface enhanced Raman scattering nanoparticles based on multiplexed Raman spectroscopy. Rahman et al., (2016), “Electromagnetic Performances Analysis of an Ultra-Wideband and Flexible Material Antenna in Microwave Breast Imaging: To Implement a Wearable Medical Bra,” Scientific Reports, 2016, Vol. 6, 38906, discloses a compact and ultra-wide band antenna on a flexible substrate for microwave imaging.

Ray et al. (2017), “A Systematic Review of Wearable Systems for Cancer Detection: Current State and Challenges,” Journal of Medical Systems, Oct. 2, 2017, 41(11), 180, reviews cancer detection using wearable systems, including sensor-based smart systems with a microcontroller, Bluetooth module, and smart phone. Robbins et al. (2021), “Two-Layer Spatial Frequency Domain Imaging of Compression-Induced Hemodynamic Changes in Breast Tissue,” Journal of Biomedical Optics, May 24, 2021, 26(5), 056005, studied hemodynamic changes in response to localized breast compression using a handheld SFDI device. Roblyer et al. (2020b), “Tracking Breast Cancer Therapies with Handheld and Wearable Diffuse Optics,” Biophotonics Congress: Biomedical Optics 2020 (Translational, Microscopy, OCT, OTS, BRAIN), OSA Technical Digest (Optical Society of America, 2020), paper TM4B.1 disclose an NIR-II imaging system), “Detection of Optically Luminescent Probes using Hyperspectral and Diffuse Imaging in Near-infrared” (DOLPHIN) for noninvasive real-time tracking of a 0.1 mm-sized fluorophore through the gastrointestinal tract of a mouse.

Saikia et al. (2017), “A Cost-Effective LED and Photodetector Based Fast Direct 3D Diffuse Optical Imaging System,” Proc. SPIE 10412, Diffuse Optical Spectroscopy and Imaging VI. Jul. 28, 2017, European Conferences on Biomedical Optics, 2017, Munich, Germany, discloses a cost-effective and high-speed 3D diffuse optical tomography system using high power LED light sources and silicon photodetectors. Saikia et al. (2019), “A Point-of-Care Handheld Region-of-Interest (ROI) 3D Functional Diffuse Optical Tomography (fDOT) System,” Proc. SPIE 10874, Optical Tomography and Spectroscopy of Tissue XIII, Mar. 1, 2019, discloses a 3D Functional Diffuse Optical Tomography (fDOT) system based on an Internet-of-things (IoT) concept. Satharasinghe et al. (2018), “Photodiodes Embedded Within Electronic Textiles,” Science Reports, 2018, 8, 16205, discloses a novel photodiode-embedded yarn with possible applications including monitoring body vital signs.

Schoustra et al. (2021), “Pendant Breast Immobilization and Positioning in Photoacoustic Tomographic Imaging.” Photoacoustics, 2021, 21, 100238 describes the design, development and added value of breast-supporting cups to immobilize and position the pendant breast in photoacoustic tomographic imaging. Shokoufi et al. (2017), “Novel Handheld Diffuse Optical Spectroscopy Probe for Breast Cancer Assessment: Clinical Study,” Journal of Biomedical Science, 6(5), 34, discloses a hand-held continuous-wave radio-frequency modulated diffuse optical spectroscopy probe. Soliman et al., (2010), “Functional Imaging Using Diffuse Optical Spectroscopy of Neoadjuvant Chemotherapy Response in Women with Locally Advanced Breast Cancer,” Clinical Cancer Research, Apr. 20, 2010, 15, 2605-2614, discloses functional imaging with tomographic near-infrared diffuse optical spectroscopy to measure tissue concentration of deoxyhemoglobin, oxyhemoglobin, percent water, and scattering power.

Spink et al. (2020), “High Optode-Density Wearable Probe for Monitoring Breast Tumor Dynamics During Neoadjuvant Chemotherapy.” Biophotonics Congress: Biomedical Optics 2020 (Translational, Microscopy, OCT, OTS, BRAIN), OSA Technical Digest (Optical Society of America, 2020), paper TTu1B.2 disclose an NIR-II imaging system), “Detection of Optically Luminescent Probes using Hyperspectral and diffuse Imaging in Near-infrared” (DOLPHIN). Spink et al. (2021), “High Optode-Density Wearable Diffuse Optical Probe for Monitoring Paced Breathing Hemodynamics in Breast Tissue,” Journal of Biomedical Optics, Jun. 2, 2021, 26(6), 062708, discloses a high optode-density wearable continuous wave diffuse optical probe for the monitoring of breathing hemodynamics in breast tissue. Tank et al. (2020), “Diffuse Optical Spectroscopic Imaging Reveals Distinct Early Breast Tumor Hemodynamic Responses to Metronomic and Maximum Tolerated Dose Regimens,” Breast Cancer Research, 2020, 22, 29 reports on a dual-center study which examined 54 breast tumors receiving NAC measured with DOSI before therapy and the first week following chemotherapy administration.

Teng (2018), “A Wearable Near-Infrared Diffuse Optical System for Monitoring in Vivo Breast Tumor Hemodynamics During Chemotherapy Infusions,” Boston University, Dissertation, 2018, discloses a new wearable diffuse optical device to investigate if very early timepoints during a patient's first chemotherapy infusion are predictive of overall response (pCR versus non-pCR) to NAC. Teng et al. (2017), “Wearable Near-Infrared Optical Probe for Continuous Monitoring During Breast Cancer Neoadjuvant Chemotherapy Infusions,” Journal of Biomedical Optics, 22(1), 14001 presents a new continuous-wave wearable diffuse optical probe for investigating the hemodynamic response of locally advanced breast cancer patients during neoadjuvant chemotherapy infusions. Tiwari et al. (2022), “Role of Sensor Technology in Detection of the Breast Cancer,” BioNanoScience, 2022, 12, 639-659, reviews different sensors developed to detect breast cancer over the past few years.

Tromberg et al., (2016), “ACRIN 6691 Investigators. Predicting Responses to Neoadjuvant Chemotherapy in Breast Cancer,” Cancer Research, Aug. 15, 2016, 76(20), 5933-5944, investigates whether changes from baseline to mid-therapy in a diffuse optical spectroscopic imaging (DOSI)-derived imaging endpoint, the tissue optical index, predict pathologic complete response in women undergoing breast cancer neoadjuvant chemotherapy. Uddin et al., (2020a), “Optimal Breast Cancer Diagnostic Strategy Using Combined Ultrasound and Diffuse Optical Tomography.” Biomedical Optics Express, 11(5), 2722-2737, presents a two-stage diagnostic strategy that is both computationally efficient and accurate. Upputuri. (2019), “Photoacoustic Imaging in the Second Near-Infrared Window: A Review,” Journal of Biomedical Optics, Apr. 9, 2019, 24(4), 040901, discusses photoacoustic (PA) imaging that combines optical excitation and ultrasound detection.

Vavadi et al., (2018), “Compact Ultrasound-Guided Diffuse Optical Tomography System for Breast Cancer Imaging.” Journal of Biomedical Optics, 2018, 24(2), 1-9, discusses an ultrasound-guided DOT system. Wang et al. (2020), “Development of a Prototype of a Wearable Flexible Electro-Optical Imaging System for the Breast,” Biophotonics Congress: Biomedical Optics 2020 (Translational, Microscopy, OCT, OTS, BRAIN), OSA Technical Digest (Optical Society of America, 2020), paper TM4B.4, discloses a wearable breast imaging system which combines a garment and a flexible electronic system. Yu et al. (2010), “Near-Infrared, Broad-Band Spectral Imaging of the Human Breast for Quantitative Oximetry: Applications to Healthy and Cancerous Breasts,” Journal of Innovative Optical Health Sciences, October 2010, 03(4):267-277 discusses the examination of ten human subjects with a previously developed instrument for near-infrared diffuse spectral imaging of the female breast.

Yuan et al. (2014), “Light-Emitting Diode-Based Multiwavelength Diffuse Optical Tomography System Guided by Ultrasound,” Journal of Biomedical Optics, Dec. 4, 2014, 19(12) 126003, discloses a low-cost DOT system using LEDs of four wavelengths in the NIR spectrum as light sources. Zhang et al., (2020), “Efficacy of Shear-Wave Elastography Versus Dynamic Optical Breast Imaging for Predicting the Pathological Response to Neoadjuvant Chemotherapy in Breast Cancer,” European Journal of Radiology, 2020, 129, 109098, discusses the value of shear-wave elastography (SWE) parameters and dynamic optical breast imaging features for predicting pathological responses to neoadjuvant chemotherapy (NACT) in breast cancer (BC).

Zhao et al. (2021), “High Resolution, Deep Imaging Using Confocal Time-of-Flight Diffuse Optical Tomography,” IEEE Transactions on Pattern Analysis and Machine Intelligence, Jul. 1, 2021, 43(7), 2206-2219, discloses how time-of-flight diffuse optical tomography (ToF-DOT) can achieve millimeter spatial resolution in the highly scattered diffusion regime. Zhao et al. (2022), “MRI-Guided Near-Infrared Spectroscopic Tomography (MRg-NIRST): System Development for Wearable, Simultaneous NIRS and MRI Imaging.” Proc. SPIE 11952, Multimodal Biomedical Imaging XVII, Mar. 2, 2022, discloses a novel wearable MRg-NIRST system for breast cancer detection with eight flex circuit strips, each with six photo-detectors (PDs) and six source fibers.

Zhu et al. (2020), “A Review of Optical Breast Imaging: Multi-Modality Systems for Breast Cancer Diagnosis,” European Journal of Radiology, August 2020, 129:109067, reviews optical breast imaging using multi-modality platforms. Zhu et al., (2021), “Early Assessment Window for Predicting Breast Cancer Neoadjuvant Therapy Using Biomarkers, Ultrasound, and Diffuse Optical Tomography,” Breast Cancer Research and Treatment, 2021, assesses the utility of tumor biomarkers, ultrasound (US) and US-guided diffuse optical tomography (DOT) in early prediction of breast cancer response to neoadjuvant therapy (NAT).

### SUMMARY OF THE INVENTION

Disclosed herein is a design for a bra cup for a smart bra which enables optical detection of abnormal breast tissue. The bra cup has a relatively-inelastic proximal portion which is closer to a person's chest wall and a relatively-elastic distal portion which is farther from the person's chest wall. There is an array of light emitters on a first side of the bra cup and an array of light receivers on a second side of the bra cup. Changes in light from the light emitters which is received by the light receivers caused by interaction between the light and breast tissue are analyzed to detect abnormal breast tissue. There is also a first set of one or more expandable chambers on the first side of the cup and a second set of one or more expandable chambers on the second side of the cup. When the expandable chambers are expanded, they gently compress the breast to improve optical scanning and detection of abnormal breast tissue.

### INTRODUCTION TO THE FIGURES

FIGS. 1 and 2 show two views of a multi-layer wearable device for optical detection of breast cancer. FIG. 1 shows this device in an unexpanded configuration. FIG. 2 shows this device in an expanded configuration.

FIGS. 1 through 6 show different views of a smart bra cup which enables optical detection of abnormal breast tissue.

FIG. 1 shows a lateral view of the cup before it has been expanded.

FIG. 2 shows a frontal view of a lateral cross-section of the cup before it has been expanded.

FIG. 3 shows the lateral view of the cup after it has been expanded.

FIG. 4 shows the frontal view of the lateral cross-section of the cup after it has been expanded.

FIGS. 5 and 6 show the frontal view of the lateral cross-section of the cup, while the breast is being optically scanned.

FIG. 7 shows the cup as part of a smart bra, wherein the central axis of the cup is vertical.

FIG. 8 shows the cup as part of a smart bra, wherein the central axis of the cup is oblique.

FIG. 9 shows a bra cup with an elliptical, oval, and/or oblate-circular shape.

FIG. 10 shows a bra cup with a teardrop shape.

FIG. 11 shows a bra cup with a football shape.

FIG. 12 shows a bra cup with a kidney-bean and/or banana shape.

FIGS. 13 and 14 show sequential views of a lateral cross-section of a cup with a partially-undulating inner component between light emitters or receivers and expandable chambers.

FIGS. 15 and 16 show sequential views of a lateral cross-section of a cup with one expandable chamber on each side.

FIGS. 17 and 18 show sequential views of a lateral cross-section of a cup with expandable chambers on only one side.

FIG. 19 shows a configuration of expandable chambers wherein each expandable chamber is connected to a separate tube.

FIG. 20 shows a configuration of expandable chambers wherein all expandable chambers on a side are connected to a common tube.

FIG. 21 shows a configuration of expandable chambers wherein a tube goes through the interiors of expandable chambers.

FIG. 22 shows a configuration wherein an expandable chamber comprises undulating sections along its length.

FIG. 23 shows a cup with an arcuate sequence of expandable chambers on each side of a virtual plane.

FIG. 24 shows a cup with two parallel sequences of expandable chambers on each side of a virtual plane.

### DETAILED DISCUSSION OF THE FIGURES

Before discussing the specific embodiments of this invention which are shown in FIGS. 1 through 24, this disclosure provides an introductory section which covers some of the general concepts, components, and methods which comprise this invention. Where relevant, these concepts, components, and methods can be applied as variations to the examples shown in FIGS. 1 through 24 which are discussed afterwards.

In an example, a bra for optical detection of abnormal breast tissue can comprise: a cup which is configured to be worn on a person's breast; wherein the cup includes a proximal portion; wherein the cup includes a distal portion, wherein the proximal portion is configured to be closer to the person's chest wall than the distal portion, and wherein the distal portion is more flexible or elastic than the proximal portion; a first set of one or more expandable chambers on a first side of a virtual plane which passes through the cup; a second set of one or more expandable chambers on a second side of the virtual plane; an array of light emitters configured to be between the first set of one or more expandable chambers and the breast; and an array of light receivers configured to be between the second set of one or more expandable chambers and the breast; wherein changes in light emitted from the light emitters and received by the light receivers which are caused by transmission of the light through the breast are analyzed to detect abnormal breast tissue.

In an example, a device can be embodied as a cup of a smart bra. In an example, a device for breast tissue imaging and/or identifying abnormal tissue in a breast can be embodied in a wearable garment (e.g. smart bra) with a plurality of light emitters and light receivers. In an example, a bra can have two cups, each of which have optical sensors and expandable chambers. In an example, a device can be embodied in a smart bra with a right-side cup and a left-side cup, each having optical sensors. In an example, right-side and left-side versions of this device can be embodied as right-side and left-side cups of a smart bra. In an example, a bra can be custom-fitted by recording the optimal expansion parameters (e.g. found by trial and error) for expandable chambers in the bra when the bra is first fitted to a person and then replicating those optimal expansion parameters when the bra is worn again. Custom-fitting the bra can enhance the quality of optical sensing, achieve results more quickly, and avoid compression-related pain. In an example, a smart bra can come in different sizes corresponding to conventional smart bra sizes.

In an example, a bra cup can have a concave shape. In an example, expandable chambers, light emitters, and light receivers can be located within the concavity of a cup in a smart bra. In an example, a bra cup can have a frustal shape, wherein there is a distal opening on a cup to allow maximum forward expansion of a breast during compression. In an example, a bra cup can have a generally hemispherical shape. In an example, the proximal perimeter of a bra cup can be substantially circular. In an example, a bra cup can have an elliptical, oval, circular, oblate-circular, and/or globular shape. In an example, a cross-section of a cup can have an elliptical or oval shape. In an example, the proximal perimeter of a bra cup can be substantially elliptical and/or oval, wherein the longest axis of the ellipse and/or oval has an oblique orientation (e.g. from the upper outer breast quadrant to the lower inner breast quadrant) (e.g. when the person wearing the bra is standing up).

In an example, a bra cup can have a teardrop-shaped perimeter. In an example, a cross-section of a cup can have a teardrop cross-sectional shape. In an example, a cup can have a teardrop shape with a vertex or point, wherein the vertex or point is configured to be placed over the Tail of Spence. In an example, a perimeter of the portion of a cup which contacts a chest wall can have a teardrop shape, wherein the apex and/or vertex of this perimeter is aligned with the Tail of Spence. In an example, an apex of a teardrop shaped cup can encompass the Tail of Spence. In an example, the perimeter of a portion of a cup which is closest to the chest wall can have a teardrop shape, wherein the vertex of the teardrop is aligned with the Tail of Spence. In an example, the proximal perimeter of a bra cup can be teardrop shaped, wherein the point of the teardrop is oriented toward the upper-outer quadrant of the breast.

In an example, the proximal perimeter of a bra cup can be substantially crescent shaped, wherein the longest axis of the crescent has a vertical orientation (e.g. when the person wearing the bra is standing up). In an example, a bra cup can have a football shape. In an example, portions of a cup which cover the upper inner, lower inner, and lower outer quadrants of the breast can have quarter-circle (e.g. quarter pie slice) cross-sectional perimeters. In an example, a portion of a cup which covers the upper outer quadrant and the Tail of Spence can have a quadrilateral cross-sectional perimeter. In an example, the diameter of the perimeter of a cup can be measured in a plane which is substantially parallel to the chest wall.

In an example, a cup can be gently adhered to the chest wall—encompassing the Tail of Spence, the upper outer quadrant, the upper inner quadrant, the lower inner quadrant, and the lower outer quadrant of a breast. In an example, the interior layer of a cup can be gently adhesive. Mild adhesion can help to keep the cup in the same position relative to breast tissue during an optical, even during expansion of the expandable chambers and/or respiratory movement. In an example, there can be a first disposable adhesive strip (e.g. half-ring) which is attached to a cup on one side of an oblique virtual plane and a second disposable adhesive strip (e.g. half-ring) which is attached to the cup on the opposite side of the oblique virtual plane.

In an example, a bra cup can be made from a soft and flexible fabric. In an example, a central portion of a cup can be more elastic (e.g. lower Young's modulus) and/or less rigid than other portions of the cup. In an example, a portion of a cup which is closer to the apex of a cup can be more elastic and/or less rigid than other portions of the cup. In an example, perimeter portions of a cup can be less elastic and/or more rigid than other portions of the cup. In an example, the center of a cup be more flexible, more elastic, and/or less rigid than the perimeter of the cup. In an example, there can be variation in the elasticity (e.g. Young's modulus), stretchability, and/or rigidity of different portions of a cup.

In an example, a bra cup with optical sensors can be made from one or more materials selected from the group consisting of: poly-Di-Methyl-Siloxane (PDMS), poly-Ethylene-Di-Oxy-Thiophene poly-Styrene Sulfonate (PEDOT:PSS), nanotubes, two-dimensional nanomaterial, Molybdenum Disulfide (MD), Thermoplastic poly-Urethane (TPU), and an multi-conjugated organic semiconductor. In an example a proximal portion of a cup can be less flexible, less elastic, and/or more rigid than a distal portion of a cup so that it resists outward expansion of the expandable chambers and directs their expansion inward toward the breast, causing gentle and partial outward flattening of the breast. In an example, a proximal portion of a cup can be less flexible and more rigid than a distal portion of a cup so that it resists outward expansion when expandable chambers are expanded.

In an example, a proximal portion of a cup can have a frustal and/or partial-conical shape. In an example, the perimeter of a cross-section of a proximal portion of a bra cup can have a football shape. In an example, the proximal portion of a cup which is closer to the chest wall can have a ring, band, column-section, or frustal shape. In an example, a proximal portion of a cup can comprise a band (e.g. cylinder or frustum) with a perimeter diameter in the range of 7 cm to 15 cm. In an example, a proximal portion of a cup can have a perimeter diameter in the range of 5 cm to 20 cm. In an example, a proximal portion of a cup can have a height (e.g. distance outward from chest wall) in the range of 3 cm to 8 cm.

In an example, a cup can comprise three layers: an inner optical layer (closest to the surface of the breast), and middle expandable layer, and an outer structural layer (farthest from the surface of the breast). In an example, an (outer) structural layer of a cup can be concave when the cup is in an expanded configuration. In an example, having some portions of a cup's structural layer provide more resistance to outward expansion than other portions can help to direct expansion of components in an expandable layer inward toward a breast so as to selectively compress the breast into a flatter configuration for better optical scanning.

In an example, a proximal portion of a bra cup can comprise a circumferential band of fabric or a textile with imbedded (or attached) circumferential wires. In an example, portions of a bra cup (e.g. of a structural layer of the cup) which are farther from a virtual plane can be reinforced with wires, but portions of the cup which are closer to virtual plane are not. In an example, proximal and distal portions of a bra cup can be made from a single (e.g. continuous) piece of material and then separately modified to change their respective levels of flexibility and/or elasticity (e.g. by embedding or attaching wires only to the proximal portion). In an example, a distal portion of a cup (farther from the chest wall) can be more flexible and/or more elastic than a proximal portion of a cup (closer to the chest wall). In an example, a distal portion of a cup which is farther from the chest wall can have a hemispherical, semi-ellipsoidal, paraboloidal, dome, or other concave shape. In an example, the perimeter of a cross-section of a distal portion of a bra cup can have a football shape. In an example, a distal perimeter of a cup can be completely open to allow maximum forward expansion of a breast during compression.

In an example, a distal portion of a cup can comprise a band (e.g. cylinder or frustum) with a perimeter diameter in the range of 4 cm to 18 cm. In an example, a distal portion of a cup can have a pre-expansion configuration before expansion of expandable chambers and outward expansion of a breast and a second post-expansion configuration after expansion of the expandable chambers and outward expansion of a breast, wherein the post-expansion configuration is at least 10% greater than the pre-expansion configuration. In an example, a distal portion of a cup can have a pre-expansion configuration before expansion of expandable chambers and outward expansion of a breast and a post-expansion configuration after expansion of the expandable chambers and outward expansion of a breast. In an example, the height or width (measured in a plane which is orthogonal and/or perpendicular to the chest wall) of a distal portion of a cup can be substantially the same as the height of the proximal portion of the cup. In an example, the height or width (measured in a plane which is orthogonal and/or perpendicular to the chest wall) of a distal portion of a cup can be between 50% and 90% of the height of the proximal portion of the cup.

In an example, a bra can comprise a plurality of hydraulic chambers and/or sections. In an example, an expandable chamber can be an air-tight compartment or pocket. In an example, an expandable chamber can be expanded by pumping water into it. In an example, an expandable chamber can be expanded by filling it with a gas (such as air), a liquid (such as water), or a gel. In an example, an expandable chamber can be expanded by a hydraulic mechanism. In an example, expandable chambers can be expanded by being filled with a gas. In an example, expandable chambers can be filled with a flowable substance (e.g. water or some other fluid).

In an example, a first expandable chamber can be in a first half of a cup which is on a first side of a virtual plane which intersects the cup and no closer than ¼ inch from the plane. In an example, a first expandable chamber can be in a first (e.g. upper) half of a cup which is on a first side of a horizontal virtual plane which intersects the cup and a second expandable chamber can be in a second (e.g. lower) half of a cup which is on a second side of the plane. In an example, a first part of an expandable layer which is on a first side of an oblique virtual plane can have more expandable chambers over the lower outer quadrant of the breast than over the lower inner quadrant of the breast. In an example, a first part of an expandable layer on a first side of a virtual plane can have the same number of expandable chambers as a second part of the expandable layer on a second (e.g. opposite) side of the virtual plane.

In an example, a first set of expandable chambers can be on one side of a virtual plane which passes through a bra cup and a second set of expandable chambers can be on the other side of this virtual plane, wherein both sets of expandable chambers are at least 1 cm away from the virtual plane. In an example, a side of an expandable chamber which faces toward an oblique virtual plane (spanning the upper out and lower inner quadrants) can be thinner than the side of the component which faces away from that plane. In an example, an expandable chamber can be in a half of a cup which is on a side of a virtual plane and no closer than ½ inch from the plane. In an example, expandable chambers can be at least 1 cm away from a virtual plane which passes through a bra cup. In an example, expandable chambers can expand predominately along (radial) vectors toward the virtual plane. In an example, first and second expandable chambers can be located to the lower left and to the upper right, respectively, of a 45-degree diagonal anterior-to-posterior virtual plane which intersects the cup.

In an example, one part of an expandable layer which is on one side of the virtual plane can have at least one expandable chamber in each quadrant spanned by the part. In an example, part of an expandable layer which is on a side of an oblique virtual plane can have at least one expandable chamber in each of: a portion of the expandable layer over the upper outer quadrant of a breast; a portion of the expandable layer over the upper inner quadrant of the breast; and a portion of the expandable layer over the lower inner quadrant of the breast. In an example, there can be a set of one or more expandable chambers on each side of a virtual plane through a bra cup. In an example, there can be a set of one or more expandable chambers on only one side (e.g. upper or lower) of a horizontal plane through a bra cup. In an example, there can be a single expandable chamber on each side of a vertical plane through a bra cup.

In an example, there can be a single expandable chamber on each side of an oblique plane through a bra cup, wherein the oblique plane spans from an upper outer quadrant of a breast to a lower inner quadrant of a breast. In an example, there can be multiple expandable chambers on each side of a vertical plane through a bra cup. In an example, there can be multiple expandable chambers on each side of an oblique plane through a bra cup, wherein the oblique plane spans from an upper outer quadrant of a breast to a lower inner quadrant of a breast. In an example, there can be three expandable chambers on each side of a virtual plane through a bra cup. In an example, there can be three expandable chambers on each side of an oblique plane through a bra cup, wherein the oblique plane spans from an upper outer quadrant of a bra cup to a lower inner quadrant of a bra cup.

In an example, a virtual plane which is between two sets of expandable chambers can be vertical when a person wearing the bra is standing (or sitting) upright. In an example, a virtual plane which is between two sets of expandable chambers can be oblique, extending from a Tail of Spence to the lower-inner quadrant of a breast. In an example, a virtual plane which passes through a cup can be oblique, wherein the oblique virtual plane is configured to span from the upper outer quadrant to the lower inner quadrant of the breast. In an example, an oblique virtual plane can be created by 45-degree rotation of a horizontal (e.g. axial) or vertical (e.g. sagittal). In an example, an oblique virtual plane which passes through a bra cup can spans from the upper outer quadrant of a breast to the lower inner quadrant of the breast. In an example, this virtual plane can be an oblique plane which spans from the upper outer quadrant (and the Tail of Spence) to the lower inner quadrant. Selectively placement of reinforcing wire in a cup can help to flatten the breast along the virtual plane to improve optical scanning.

In an example, a cup can comprise a first expandable chamber on (or in) the upper left quadrant (from a frontal corona view) of the cup for a left-side breast and a second expandable chamber on (or in) the lower right quadrant (from a frontal corona view) of the interior of the cup. In an example, the majority of expandable chambers can be located interior to the proximal portion of the bra cup, with the remaining expandable chambers located interior to the distal portion of the bra cup. In an example, there can be three expandable chambers on each side of the virtual plane.

In an example, a portion of the breast between the expandable chambers can be flatter in a second configuration than in a first configuration of the cup. It is anticipated that breast compression from this smart bra will be less uncomfortable than compression in traditional mammography because: uniform breast flatness is not as critical in optical scanning as in traditional mammography; and the bra cup does not compress a breast with rigid rectangular surfaces as in traditional mammography. In an example, expandable chambers can be expanded until a minimum target level and/or percentage of light emitted from light emitters is received by light receivers after passing through breast tissue. In an example, expansion of one or more expandable chambers can be adjusted and/or controlled to achieve a desired width of the breast without undue pressure.

In an example, an expandable chamber can be shaped like a quarter of a circle, ring, or torus. In an example, an expandable chamber can have a crescent, partial moon, and/or banana shape. In an example, an expandable chamber can have a disk shape in a first configuration and a cylindrical shape in an expanded second configuration. In an example, an expandable chamber can have a shape which is selected from the group consisting of: pancake, disk, ellipsoidal, oblong, oval, toroidal, hemispherical, and spherical. In an example, an expandable chamber can have undulating (e.g. width-varying and/or sinusoidal) sections along its length.

In an example, expandable chambers can comprise an undulating (e.g. alternatingly narrow and wide) sequence of connected fluid (e.g. gas or liquid) filled chambers. In an example, there can be a single crescent or banana shaped expandable chamber on each side of an oblique plane through a bra cup, wherein the oblique plane spans from an upper outer quadrant of a breast to a lower inner quadrant of a breast. In an example, there can be a single crescent or banana shaped expandable chamber on each side of an oblique plane through a bra cup. In an example, expandable chambers in portions of a cup which cover the lower outer and upper inner quadrants of the breast can be less triangular and/or more trapezoidal than expandable chambers in other portions of the cup. In an example, a plurality of expandable chambers can span the entire perimeter of a cup. In an example, a plurality of expandable chambers can span between 60% and 85% of the perimeter of a cup. In an example, expandable chambers on a first side of a virtual plane can have substantially the same sizes and/or shapes as expandable chambers on a second (e.g. opposite) side of the virtual plane.

In an example, a first expandable chamber can be expanded by a greater percentage than a second expandable chamber. In an example, expandable chambers and/or sections which are closer to the center of a cup can be expanded to a greater extent than components and/or sections which are farther from the center of the cup. In an example, expandable chambers in portions of a cup which cover the lower outer and upper inner quadrants of the breast can be larger than expandable chambers in other portions of the cup. In an example, expandable chambers in the expandable layer can be expanded to different extents, to different sizes, and/or to different internal pressure levels. In an example, expandable chambers which are closer to the center of the cup can be expanded more than expandable chambers which are farther from the center of the cup.

In an example, a cup can have a sufficiently large number of individually-controllable expandable chambers that it can cause arrays of light emitters and receivers to conform to the shape of a particular breast with a specific size and shape, thereby reducing (or even eliminating) air gaps between the light emitters and receivers and the surface of the breast. In an example, an expandable layer can comprise a plurality of inflatable chambers which can be individually and selectively inflated so that they are inflated by different degrees, by different amounts, to different sizes, and/or to different internal pressures, thereby compressing different portions of the breast by different extents. In an example, each expandable chamber can be connected to a separate tube. This enables independent (e.g. differential and/or sequential) expansion of different (e.g. selected) expandable chambers.

In an example, expandable chambers can be sufficiently controllable and flexible that they can greatly reduce (or even eliminate) air gaps between the optical layer and the surface of the breast, even for breasts with different sizes and shapes. In an example, hydraulic chambers can be individually and selectively expanded by different degrees, by different amounts, to different sizes, and/or to different internal air pressures. In an example, inflatable chambers can be individually and selectively inflated so that they are inflated by different degrees, by different amounts, to different sizes, and/or to different internal pressures, thereby exerting different levels of pressure on different portions of the breast. In an example, different expandable chambers in a set of expandable chambers can be expanded to different levels of internal pressure.

In an example, a side of an expandable chamber which faces toward a breast can be more elastic than a side of the component which faces away from the breast. In an example, having one side of an expandable chamber be more elastic than the other can help to direct expansion of the expandable chamber toward the surface of a breast. In an example, the side and/or surface of an expandable chamber which faces away from the perimeter of a cup can have a lower Young's modulus than the side of the component which faces toward the perimeter of the cup. In an example, the side of an expandable chamber which faces toward an oblique virtual plane (spanning the upper out and lower inner quadrants) can be more elastic than the side of the component which faces away from that plane. In an example, there can be multiple expandable chambers on each side of a virtual plane through a bra cup, wherein a second expandable chamber is closer to the center of the bra cup than a first expandable chamber, and wherein the second expandable chamber has a less-elastic perimeter layer than the first expandable chamber.

In an example, having one side of an expandable chamber be thinner than the other can help to direct expansion of the expandable chamber toward the surface of a breast. In an example, different expandable chambers in a set of expandable chambers can be expanded (e.g. inflated) at different times and/or in specific sequence. In an example, a compressive wave via a plurality of expandable chambers can achieve more-uniform post-compression tissue width and/or help to avoid pain during breast compression. In an example, expandable chambers in the expandable layer can be expanded at different times and/or in a sequential manner. In an example, expansion of expandable chambers in a sequential manner can create peristaltic motion which causes a wave of compression of breast tissue.

In an example, a bra can include tubes which conduct a flowable substance (e.g. air or water) from a pump to expandable chambers, wherein these tubes are imbedded in (or attached to) in a back strap of the bra. In an example, a smart bra can further comprise tubes or channels which conduct a flowable substance (e.g. a gas or liquid) into a plurality of expandable chambers. In an example, expandable chambers can be expanded by being filled with a flowable substance which is inserted into them through these tubes. In an example, this flowable substance can be a gas (e.g. air) or a liquid (e.g. water).

In an example, a bra can include one or more tubes which deliver a flowable substance (e.g. air or water) into the one or more expandable chambers. In an example, there can be a common flowable-substance tube which passes through all expandable chambers in a given set of expandable chambers (e.g. all expandable chambers on the same side of a virtual plane). In an example, there can be a common flowable-substance tube which is connected to all expandable chambers. In an example, there can be a separate air tube or channel for each expandable chamber. In an example, a bra can include an air pump which pumps air into the one or more expandable chambers. In an example, a pump can be separate, removably-connected to the bra for expansion of the expandable chambers, and detached for wearing the bra.

In an example, a person wearing the bra can control the amount of pressure exerted by expansion of expandable chambers on their breast by manually pumping a flowable substance (e.g. air or water) into the expandable chambers. In an example, expandable chambers can be expanded by a manual pump which is operated by the person wearing the bra so that that person can easily avoid pain from over-compression by stopping expansion. In an example, expandable chambers can be automatically expanded by an automatic pump which pumps a flowable substance (e.g. air or water) into the chambers.

In an example, a bra cup can include one or more pressure sensors which measure the pressure and/or force applied to a breast by expansion of expandable chambers. In an example, a compression-monitoring mechanism can comprise monitoring of the amount of light which is transmitted through the breast, especially the widest portion of the breast. In an example, a smart bra can further comprise a compression-monitoring mechanism to monitor the amount of breast compression from expansion of the expandable level to ensure sufficient compression to get good analysis of breast tissue from transmission of light through the breast, but not so much compression than it causes pain, circulation problems, or other adverse effects. In an example, expansion of expandable chambers can be controlled (e.g. limited) based on pressure levels on those chambers. In an example, pressure sensors can measure the amount of pressure applied by a cup to breast tissue to help avoid undue breast compression and discomfort. In an example, a smart bra can include pressure sensors which control a pump, wherein expansion is stopped when pressure levels reach a selected level. In an example, a bra cup can include one or more motion and/or bend sensors which measure the amount (e.g. relative distance) by which expansion of expandable chambers has compressed a breast in order to avoid uncomfortable levels of compression.

In an example, a light emitter can be a laser with a narrow pulse width. In an example, a light emitter can be a MicroLED. In an example, a light emitter can be a Nanoscale LED. In an example, a light emitter can be a Resonant Cavity Light Emitting Diode (RCLED). In an example, a light emitter can be a Super-Luminescent Light Emitting Diode (SLED). In an example, a light emitter can be an Organic Photovoltaic (OPV). In an example, light emitters can be continuous wave light emitters. In an example, light emitters can emit coherent light. In an example, one or more light emitters in a cup can be Super-Luminescent Light Emitting Diodes (SLEDs). In an example, one or more light emitters in a cup can be red-light lasers. In an example, one or more light emitters in a cup can be multi-wavelength lasers. In an example, one or more light emitters in a cup can be Light Emitting Diodes (LEDs). In an example, one or more light emitters in a cup can be coherent light emitters. In an example, one or more light emitters in a cup can be Light-Emitting Electrochemical Cells (LECs).

In an example, a first light emitter can emit intensity or amplitude modulated light into the breast with a wavelength in the range of 650 to 750 nm; a second light emitter can emit intensity or amplitude modulated light with a wavelength in the range of 750 nm to 850 nm; and a third light emitter can emit intensity or amplitude modulated light with a wavelength in the range of 850 nm to 950 nm. In an example, a first light emitter can emit light with a wavelength in the range of 650 to 750 nm; a second light emitter can emit light with a wavelength in the range of 750 nm to 850 nm; and a third light emitter can emit light with a wavelength in the range of 850 nm to 950 nm. In an example, a first light emitter can emit light with a wavelength in the range of 600 to 900 nm; a second light emitter can emit light with a wavelength in the range of 900 nm to 1200 nm; and a third light emitter can emit light with a wavelength in the range of 1200 nm to 1500 nm.

In an example, a first light emitter can emit light with a wavelength in the range of 600 to 900 nm at a first time and a second light emitter can emit light with a wavelength in the range of 900 nm to 1200 nm at a second time. In an example, a first light emitter can emit light with a wavelength in the range of 600 to 700 nm at a first time; a second light emitter can emit light with a wavelength in the range of 700 nm to 800 nm at a second time; and a third light emitter can emit light with a wavelength in the range of 800 nm to 900 nm at a third time. In an example, a first set of light emitters can emit light at a first frequency and/or wavelength (or in a first spectral range) and a second set of light emitters can emit light at a second frequency and/or wavelength (or in a second spectral range).

In an example, a light emitter can emit light at different wavelengths over time, within the range of 600 to 1100 nm. In an example, a light emitter can emit light at a frequency and/or wavelength which varies over time. In an example, a light emitter can emit light with a wavelength and/or frequency which changes over time. In an example, different light emitters in a cup can emit light with different wavelengths, within the range of 600 to 1100 nm. In an example, different light emitters in an array of light emitters can emit light in different ranges of the light spectrum. In an example, light emitters can emit light at a frequency and/or wavelength which varies over time. In an example, light emitters in a cup can emit light with one or more wavelengths, within the range of 600 to 1100 nm. In an example, light emitters in a cup can emit light at different wavelengths over time selected from the group consisting of: 600, 650, 660, 680, 690, 750, 775, 780, 785, 800, 808, 810, 830, 850, and 1000 nm. In an example, one or more light emitters in a cup can be ultraviolet light emitters. In an example, the wavelengths and/or frequencies of light from light emitters can be changed in periodic manner.

In an example, an array of light emitters can comprise an array of nested (e.g. concentric and/or coaxial) arcs, wherein light emitters are located along the arcs. In an example, light emitters can be configured in concentric (e.g. nested) half rings. In an example, light emitters can be configured in a spiral array. In an example, light emitters can be configured in a helical array. In an example, light emitters can be configured along undulating (e.g. sinusoidal) rings around a breast. In an example, light emitters can be evenly spaced along longitudinal lines around a breast. In an example, light emitters in a cup can be arranged in a hub-and-spoke configuration. In an example, light emitters in a cup can be arranged in (e.g. distributed along) an orthogonal mesh, grid, and/or matrix.

In an example, light emitters can be closer together toward the apex of a cup and farther apart toward the periphery of the cup. In an example, light emitters can be farther apart toward the apex of a cup and closer together toward the periphery of the cup. In an example, light emitters in the portion of a cup which covers the lower outer quadrant of the breast can be closer together than light emitters in other portions of the cup. In an example, light emitters which are closer to the chest wall can be farther apart than light emitters which are farther from the chest wall. In an example, the density of light emitters on a cup can be greater (and/or the distance between light emitters can be less) for portions of the cup which are farther from the center (and/or apex) of the cup than for portions of the cup which are closer to the center (and/or apex) of the cup.

In an example, the density of light emitters in the portion of a cup covering the upper outer quadrant and Tail of Spence of a breast can be greater (and/or the distance between light emitters can be less) than for the portion of the cup covering the lower inner quadrant of the breast. In an example, the distances between light emitters and detectors in the portion of a cup covering the upper outer quadrant and Tail of Spence of a breast can be greater than for the portion of the cup covering the lower inner quadrant of the breast. In an example, there can be a first average distance between light emitters and light receivers when a cup is in its first (unexpanded) configuration and a second average distance between light emitters and light receivers when the cup is in its second (expanded) configuration, wherein the second average distance is less than the first average distance. In an example, a cup can have an optical layer comprising a plurality of light emitters and light receivers. In an example, at least some light emitters can be between expandable chambers and the breast. In an example, light emitters can be encapsulated in acrylic material for protection from moisture. In an example, light emitters can be printed on a fabric or textile.

In an example, an array of light emitters can be to the right or to the left of a vertical plane which separates a first set of expandable chambers from a second set of expandable chambers. In an example, an array of light emitters can be above or below a horizontal plane which separates a first set of expandable chambers from a second set of expandable chambers. In an example, light emitters which are closer to an oblique virtual plane spanning the upper outer quadrant and the lower inner quadrant can be farther apart than light emitters which are farther from this virtual plane. In an example, there can be an array of light emitters to one side of a virtual plane which passes through a bra cup, wherein there are between 5 and 20 light emitters in the array.

In an example, a light emitter can be oriented to emit light along a vector which is substantially perpendicular to the closest surface of a breast. In an example, a light emitter can be positioned so as to emit light along a vector which is substantially perpendicular to a breast surface and/or directed toward a particular light receiver. In an example, light emitters can be configured to direct light into breast tissue along vectors which are substantially orthogonal to the breast surface. In an example, a light emitter can emit a radially-rotating beam of light. In an example, the angles at which an array of light receivers receive light from breast tissue can be changed (e.g. controlled) by an array of movable micromirrors.

In an example, a light emitter can emit a first pulse of light with a first duration followed by a second pulse of light with a second duration, wherein the second duration is greater than the first duration. In an example, a light emitter can emit light with a wavelength and/or frequency which changes in a repeated cyclical pattern over time. In an example, different light emitters in an array of light emitters can emit light at different times. In an example, light emitters can all emit a pulse of light at the same time. In an example, light emitters in a first quadrant can emit (a pulse of) light at a first time and light emitters in a second quadrant can emit (a pulse of) light at a second time. In an example, light emitters in an array of light emitters can be activated in a linear sequence. In an example, light emitters on the top half of a cup can emit (a pulse of) light at a first time and light emitters on the bottom half of the cup can emit (a pulse of) light at a second time, or vice versa In an example, light emitters one a first of light emitters can emit (a pulse of) light at a first time and light emitters on a second ring of light emitters can emit (a pulse of) light at a second time.

In an example, a light receiver can be a photoconductor. In an example, a light receiver can be a photoreceptor. In an example, a light receiver can be an organic phototransistor. In an example, a light receiver can be made from silver nanowires. In an example, a light receiver can be made from Germanium. In an example, a light receiver can be selected from the group consisting of: photodetector, photoresistor, avalanche photodiode (APD), charge-coupled device (CCD), complementary metal-oxide semiconductor (CMOS), infrared detector, infrared photoconductor, infrared photodiode, light dependent resistor (LDR), optoelectric sensor, photoconductor, photodiode, photomultiplier, and phototransistor. In an example, a light receiver can have an organic photoactive channel layer, a dielectric layer, and electrodes. In an example, a light receiver can be made with polyethylene naphthalate. In an example, a light receiver can be made from polydimethylsiloxane (PDMS).

In an example, an array of light receivers can comprise an array of nested (e.g. concentric and/or coaxial) rings, wherein light receivers are located around the rings. In an example an array of light receivers can be configured in concentric (e.g. nested) rings. In an example an array of light receivers can be configured in a star-burst array. In an example an array of light receivers can be configured in a honeycomb array (e.g. hexagonal grid or mesh). In an example an array of light receivers can be configured in a checkerboard array. In an example, an array of light receivers can comprise an array of nested (e.g. concentric and/or coaxial) semicircles, wherein light receivers are located along the semicircles. In an example, an array of light receivers can be arranged in (e.g. distributed along) rings. In an example, an array of light receivers can be arranged in (e.g. distributed along) a hexagonal (e.g. honeycomb) mesh, grid, and/or matrix.

In an example, light receivers in the portion of a cup which covers the upper outer quadrant and the Tail of Spence of the breast can be closer together than light receivers in other portions of the cup. In an example, light receivers which are farther from an oblique virtual plane spanning the upper outer quadrant and the lower inner quadrant can be farther apart than light receivers which are closer to this virtual plane. In an example, light receivers which are closer to an oblique virtual plane spanning the upper outer quadrant and the lower inner quadrant can be farther apart than light receivers which are farther from this virtual plane. In an example, the density of light receivers in the portion of a cup covering the upper outer quadrant of a breast can be less (and/or the distance between light receivers can be greater) than for the portion of the cup covering the lower inner quadrant of the breast. In an example, the density of light receivers in a cup can be greater (and/or the distance between light receivers can be less) for portions of the optical layer which are farther from the center (and/or apex) of the cup than for portions of the optical layer which are closer to the center (and/or apex) of the cup.

In an example, light receivers can be located directly on the perimeters of expandable chambers. In an example, an array of light receivers can be on one side of a virtual plane which passes through a bra cup, wherein on one side of a virtual plane means being in space which is on one side of the virtual plane, not being located directly on the side of the virtual plane. For example, the array of light receivers can be on one side of a virtual plane, but still be centimeters away from the virtual plane. As used herein, the phrase on one side of can also be interpreted as to one side of.

In an example, a cup can comprise light emitters and light receivers which are in direct contact with the surface of a breast. In an example, an optical module can include one light receiver and at least four light emitters which are evenly-distributed around the light receiver. In an example, an optical module can include one light receiver and a plurality of light emitters distributed around the light receiver. In an example, an optical module can include one light emitter and a plurality of light receivers which are evenly-distributed around the light emitter. In an example, light emitters and light receivers can be on opposite sides of a breast.

In an example, a bra for optical detection of abnormal breast tissue can comprise an optical layer with a plurality of light emitters and light receivers, wherein a first part of the optical layer is on the first side of the virtual plane and a second part of the optical layer is on the second side of the virtual plane. In an example, light emitted from the light emitters on the first side of a virtual plane can be received by light receivers on a second side of the virtual plane. In an example, optical components, which each comprise one or more light emitters and one or more light receivers, can be distributed on both sides of a virtual plane. In an example, a layer of light emitters and receivers can span between 60% and 85% of the interior concavity of a cup. In an example, a layer of light emitters and receivers can span all of the interior concavity of a cup.

In an example, a bra cup can include a component (e.g. layer of material) between light receivers and expandable chambers. In an example, a bra cup can include a rigid component between expandable chambers and light receivers. In an example, an inner component (e.g. an inner ring or layer) between light receivers and expandable chambers can comprise: a first portion on a first side of the virtual plane, a second portion on a second side of the virtual plane, and two undulating and/or pleated portions which cross the virtual plane. In an example, an inner component (e.g. inner ring or band) between light emitters or receivers and expandable chambers can have undulating and/or pleated portions.

In an example, there can be a concave component between expandable chambers and a breast, wherein light receivers are imbedded within this component. In an example, there can be a concave component between expandable chambers and a breast, wherein light emitters are imbedded within this component. In an example, there can be a layer of material between expandable chambers and light emitters. In an example, there can be an arcuate layer of material between expandable chambers and light receivers, wherein this is on one side of the virtual plane and spans across multiple expandable chambers. In an example, there can be an arcuate layer of material between expandable chambers and light emitters, wherein this layer spans multiple expandable chambers.

In an example, a cup can comprise light emitters, light receivers, and electroconductive pathways which are printed on an elastomeric substrate. In an example, a cup can have a plurality of elastic electroconductive pathways which are configured in nested (e.g. concentric) rings. In an example, a cup can have a plurality of elastic electroconductive pathways which are configured in a star burst pattern. In an example, a cup can have a plurality of elastic electroconductive pathways which are configured in a radial strips which extend outward from the apex of a cup. In an example, a cup can have a plurality of elastic electroconductive pathways which are configured in a helical or half-helical pattern. In an example, an optical layer can further comprise flexible electroconductive pathways which are in electrical communication with light emitters and light receivers.

In an example, electroconductive pathways in a cup can be made with a silicone-based polymer which has been doped, impregnated, and/or coated with electroconductive material. In an example, electroconductive pathways in a cup can be made with poly(3,4-ethylenedioxythiophene) polystyrene sulfonate (PEDOT:PSS). In an example, flexible electroconductive pathways can be made from an elastomeric polymer (such as PDMS) which has been impregnated with carbon nanotubes. In an example, light emitters can receive electrical power through undulating wires which are embedded in an elastomeric polymer. In an example, light emitters can receive electrical power through carbon nanotubes in an elastomeric polymer (e.g. PDMS). In an example, these electroconductive pathways can be undulating and/or elastic so that they do not constrain expansion of expandable chambers.

In an example, a cup can comprise a plurality of endpoints (or side locations) on a plurality of optical fibers which emit light, wherein these optical fibers transmit this light from light sources (e.g. LEDs) around the chest-wall perimeter of the optical layer. In an example, a cup can further comprise optical fibers which guide light from a plurality of light emitters to a plurality of light-exiting points. In an example, a cup can further comprise undulating (e.g. sinusoidal) optical fibers which guide light to light-exiting points. In an example, optical fibers in a cup can transmit light which originates from light sources (e.g. LEDs) outside the cup.

In an example, a cup can comprise an air-gap-reducing layer, wherein a first part of the layer spans the lower inner quadrant, the lower outer quadrant, the upper outer quadrant, and the Tail of Spence of a breast. In an example, a first part of an air-gap-reducing layer can be on one side of a virtual plane and a second part of the air-gap-reducing layer can be on the opposite side of the virtual plane. In an example, a wearable cup for optical scanning of breast tissue can comprise three layers: an air-gap-reducing inner layer, an optical layer containing light emitters and receivers, and a structural outer layer. In an example, an air-gap-reducing layer can enable light from light emitters in the optical layer to enter breast tissue with minimal refraction or scattering from air gaps.

In an example, an air-gap-reducing layer of a cup can be made with a silicone-based material such as polydimethylsiloxane (PDMS). In an example, an air-gap-reducing layer of a cup can be made from a xerogel. In an example, an air-gap-reducing layer of a cup can be made from a PDMS-hydrogel composite. In an example, an air-gap-reducing layer of a cup can be made from a cryogel. In an example, an air-gap-reducing layer of a cup can be made from fibrin. In an example, an air-gap-reducing layer of a cup can be made from poly-vinyl Chloride Plastisol (PVCP). In an example, an air-gap-reducing layer of a cup can be made from polyurethane (PU). In an example, an air-gap-reducing layer of a cup can be made from a copolymeric polymer gel. In an example, an air-gap-reducing layer of a cup can be made from polyethylene Glycol (PEG). In an example, an air-gap-reducing layer of a cup can be made from poly-acrylo-nitrile (PAN).

In an example, an air-gap-reducing layer of a cup can be transparent. In an example, an air-gap-reducing layer of a cup can contain a gel. In an example, an air-gap-reducing layer of a cup can have an optical scattering coefficient with a value like the average value for normal breast tissue. In an example, an air-gap-reducing layer of a cup can have an optical absorption coefficient with a value like the average value for normal breast tissue. In an example, an air-gap-reducing layer of a cup can have an anisotropy factor with a value like that of breast tissue. In an example, an air-gap-reducing layer of a cup can have a Shore durometer level which is less than 30. In an example, central portions of first and second parts of an air-gap-reducing layer can be moved a greater distance than non-central portions of the first and second parts when an expanding layer is expanded and a cup is changed from its first configuration to its second configuration.

In an example, the air-gap-reducing layer of a cup can be transparent. In an example, the inner air-gap-reducing layer of a cup can comprise a conformable gel which is either transparent or has optical qualities which are similar to those of normal breast tissue. In an example, the thickness, elasticity, and/or compressibility of an air-gap-reducing layer can be adjusted by changing the pressure of a gas inside the layer. In an example, the thickness, elasticity, and/or compressibility of an air-gap-reducing layer can be adjusted in order to better conform the layer to the shape and size of a breast.

In an example, a bra can comprise one or more other components selected from the group consisting of: power source, data processor, wireless data transmitter, wireless data receiver, air pump, and liquid pump. In an example, a smart bra can further comprise one or more other components selected from the group consisting of: power source (e.g. battery), flexible electroconductive wires and/or textile channels, data processor (local, remote, or both local and remote), wireless data transmitter, wireless data receiver, pressure sensors, motion sensors (e.g. accelerometer and gyroscope), inclinometer, mirrors (e.g. micromirror array), pump and/or impellor (e.g. air or liquid pump or impellor), tubes (e.g. air or liquid conducting tubes), air or liquid reservoir, and electromagnetic actuator. In an example, one or more components such as a power source, data processor, data transmitter/detector, and pump can be located on the posterior portion (e.g. the back strap) of a bra.

In an example, a bra can include a data processing unit which controls the operation of the expandable chambers. In an example, a smart bra can be a component of a system which includes a local data processor, a local data transmitter (which are contiguous parts of the smart bra) and a remote (non-contiguous) data processor which receives data from the data transmitter. In an example, a smart bra can further comprise a local data processor which is located on the back strap. In an example, a smart bra can further comprise a data processor which controls the operation of an air pump or liquid pump.

In an example, a bra can include a data transmitter which transmits data from the light receivers to a remote data processor, wherein this data is analyzed in the remote data processor to detect abnormal breast tissue. In an example, a system can comprise a local data processor and data transmitter which are part of a smart bra and a remote data processor which receives data from the local data processor via the data transmitter. In an example, data from a smart bra can be wirelessly transmitted to a data processor in a different wearable device (e.g. a smart watch), a handheld device (e.g. a cell phone), or a remote server (e.g. battery). In an example, a bra can include an energy transducer which generates electricity from body heat. In an example, a power source (e.g. battery) can be located on a posterior portion of a smart bra.

In an example, a device with optical sensors to detect abnormal breast tissue can be a bra insert which is inserted between the cup of a conventional bra (e.g. bra cup) and a breast. In an example, a device with optical sensors to detect abnormal breast tissue can be bra insert. In an example, a device with optical sensors to detect abnormal breast tissue can be separate from a bra. In an example, a device with optical sensors to detect abnormal breast tissue can be inserted between a bra cup and a breast. In an example, a device with optical sensors to detect abnormal breast tissue can be removably-attached to the concave interior of a bra cup by an attachment mechanism (e.g. hook-and-loop material, snap, clip, or magnet) so that it is held in place for optical scanning, but can also be removed for washing the bra without exposing optical sensors (or other electronics) to water and soap. In an example, a device with optical sensors to detect abnormal breast tissue can be an adhesive patch, sticker, or bandage. In an example, a device with optical sensors to detect abnormal breast tissue can be embodied in a flexible patch, bandage, or sticker which is attached to a breast.

In an example, a modular device (e.g. bra insert) can be used on the right-side and then on the left-side of a conventional bra. In an example, one or more of these components can be temporarily removed from a bra so that the rest of the bra can be washed. In an example, a bra and/or cup can further comprise one or more marks, indicators, openings, or sensors which help to register (e.g. identify) its location relative to a selected location, anatomical feature, or orientation of a breast. In an example, a bra and/or cup can have marks or openings which are aligned anatomical features on a person's body to register its location and ensure placement alignment during repeated uses at different times. This can be useful for tracking possible changes in specific locations of breast tissue over time.

In an example, a cup or cup insert can be rotated for scanning from a different angle so that the same light emitters and light receivers are on either side of a vertical plane, with the upper and lower outer quadrants on one side of the plane and the upper and lower inner quadrants on the other side of the plane. In an example, a modular insert inserted between a bra cup and a breast can be rotated and/or flipped between being used on a right breast and being used on a left breast. Such rotation and/or flipping can enable it to be designed with to provide more accurate scanning of the upper outer quadrant and/or Tail of Spence because this area has a higher probability of developing malignant tissue. In an example, one or more layers of a cup or a cup insert can be rotated to change the orientation of a virtual plane and to compress the breast from different angles. In an example, one or more portions of a cup or cup insert can be rotated relative to other portions of a bra in order to adjust the portions of the breast which are compressed and/or optically scanned.

In an example, changes in light caused by interaction with breast tissue can be used to identify attributes of abnormal breast tissue such as location, size, shape, and composition. In an example, a smart bra can detect and/or image abnormal tissue by analyzing light transmission. In an example, changes in the direction of light caused by its transmission through breast tissue can be analyzed to identify the levels, concentrations, and/or locations of specific biological substances (e.g. markers associated with abnormal tissue) in the breast tissue. In an example, changes in the intensity (e.g. amplitude) of light emitted from light emitters and received by light receivers which are caused by transmission of the light through breast tissue can be analyzed to evaluate the molecular composition of breast tissue and detect abnormal breast tissue. In an example, changes in the intensity of light caused by its transmission through breast tissue can be analyzed to identify the levels, concentrations, and/or locations of specific biological substances (e.g. markers associated with abnormal tissue) in the breast tissue. In an example, light from the light emitters can be received by the light receivers after it has passed through breast tissue.

In an example, changes in light reflected from breast tissue can be analyzed to detect and/or image abnormal tissue. In an example, changes in the intensity of light caused by its reflection from breast tissue can be analyzed to identify the levels, concentrations, and/or locations of specific biological substances (e.g. markers associated with abnormal tissue) in the breast tissue. In an example, light from the light emitters which has been transmitted through and/or reflected from breast tissue and received by the light receivers can be analyzed to detect and/or image abnormal breast tissue.

In an example, a smart bra can detect abnormal tissue via one or more optical scanning methods selected from the group consisting of: Diffuse Optical Imaging (DOI). Diffuse Optical spectroscopic Imaging (DOSI), Diffuse Optical spectroscopy (DOS), Diffuse Optical Tomography (DOT), Frequency-Domain Photon Migration (FDPM), Functional Near-Infrared spectroscopy (fNIRS), Near-Infrared spectroscopy (NIRS), Raman spectroscopy, reflectance Diffuse Optical Tomography (RDOT), Transillumination Imaging (TI), and/or Transmittance Diffuse Optical Tomography (TDOT).

In an example, a smart bra can use Diffuse Optical Tomography (DOT) to analyze the molecular composition of breast tissue, detect abnormal breast tissue, evaluate the size and shape of abnormal breast tissue, identify selected biometric parameters in breast tissue, identify the location of abnormal breast tissue, and/or image breast tissue. In an example, a smart bra can use time of flight Diffuse Optical Tomography (DOT) to analyze the molecular composition of breast tissue, detect abnormal breast tissue, evaluate the size and shape of abnormal breast tissue, identify selected biometric parameters in breast tissue, identify the location of abnormal breast tissue, and/or image breast tissue.

In an example, changes in amplitude and/or spectrum of light from the light emitters which are caused by reflection of the light by breast tissue and/or transmission of the light through breast tissue can be analyzed to image breast tissue and/or detect breast cancer. In an example, changes in the intensity and/or spectral distribution of light caused by transmission through breast tissue can be analyzed to create a (3D) image which shows the locations, sizes, and shapes of abnormal breast tissue. In an example, changes in the intensity and/or spectral distribution of light caused by transmission through breast tissue can be analyzed to create a (3D) image which shows levels and/or concentrations of biological substances (e.g. markers) which are associated with abnormal tissue.

In an example, changes in the spectrum (e.g. spectral distribution) of light emitted from light emitters and received by light receivers which are caused by transmission of the light through breast tissue can be analyzed to image breast tissue. In an example, changes in the spectrum of light caused by its transmission through breast tissue can be analyzed to identify the levels, concentrations, and/or locations of specific biological substances (e.g. markers associated with abnormal tissue) in the breast tissue. In an example, changes in the spectrum of light caused by its reflection from breast tissue can be analyzed to create an image of the breast tissue.

In an example, spectroscopic analysis of near-infrared light which has been transmitted through and/or reflected from breast tissue can be done to identify the sizes, configurations, and/or locations of specific biological structures associated with abnormal tissue. In an example, spectroscopic analysis of near-infrared light which has been reflected from breast tissue can be done to identify the levels, concentrations, and/or locations of specific biological substances (e.g. markers associated with abnormal tissue) in the breast tissue. In an example, spectroscopic analysis of light which has been transmitted through breast tissue can be done to identify the levels, concentrations, and/or locations of specific biological substances (e.g. markers associated with abnormal tissue) in the breast tissue.

In an example, light which has been transmitted through and/or reflected from breast tissue can be analyzed to detect and/or image the locations and configurations of lymphatics in breast tissue. In an example, light which has been transmitted through and/or reflected from breast tissue can be analyzed to detect and/or image levels, concentrations, and/or locations of water in breast tissue. In an example, light which has been transmitted through and/or reflected from breast tissue can be analyzed to detect and/or image levels, concentrations, and/or locations of lipids in breast tissue.

Although light energy is significantly scatter and/or diffused through the depth of breast tissue, joint three-dimensional analysis of light transmitted through multiple intersecting vectors between multiple pairs of light emitters and light receivers can provide parallel pathway data which increases the accuracy and locational precision of spectroscopic analysis in order to identify and locate abnormal tissue. Joint analysis of the intensity and spectral changes of light beams traveling through the breast tissue along different three-dimensional vectors can identify whether there is abnormal tissue within the breast and, if so, where the abnormal tissue is located.

In an example, a smart bra can be a particularly good for periodic imaging of breast tissue and tracking longitudinal changes because the bra tends to place light emitters and receivers over the same locations on a breast each time the bra is worn. This is an advantage over hand-held devices which are unlikely to scan a breast along the same paths and same locations in periodic scans at different times. In an example, changes in tissue composition over time can be identified which could indicate abnormal tissue growth.

In an example, a device can be embodied as a smart bra. In an example, a cup on one side (e.g. the right side or left side) of a smart bra can have a similar (e.g. same, but symmetric) configuration of expanding components, light emitters, and light receivers as a cup on the other side (e.g. the left side or right side) of the smart bra. In an example, a device can be embodied in a smart bra with a right-side cup and a left-side cup, each having light emitters and light receivers. In an example, the results from optically-scanning right and left breasts can be compared and/or contrasted to each other to help detect abnormal breast tissue.

In an example, a bra can identify the optimal compression profile for a specific breast (e.g. customized for the right or left breast of a specific person), wherein this compression profile includes expansion parameters for expandable chambers in the bra. In an example, the size of an interior concavity of a smart bra cup can correspond to the cup size of a conventional smart bra, but the exterior size of the smart bra cup can be larger due to space occupied by expandable chambers, light emitters, and light receivers.

In an example, a bra cup can encompass the Tail of Spence, the upper outer quadrant, the upper inner quadrant, the lower inner quadrant, and the lower outer quadrant of a breast. In an example, a cup can have a concave shape. In an example, a bra cup can have a frustal shape, wherein the distal perimeter of a cup can be completely open to allow maximum forward expansion of a breast during compression. In an example, an (outer) structural layer of a cup can have a shape which is selected from the group consisting of: hemisphere, half of an oblate spheroid, half of an ellipsoid, half of a 3D teardrop shape, and conic section. In an example, a bra cup can have an elliptical, oval, and/or oblate-circular shape. In an example, the proximal perimeter of a bra cup can be substantially elliptical and/or oval.

In an example, a bra cup can have a teardrop-shaped perimeter, wherein the point of the teardrop is oriented toward the upper-outer quadrant of the breast. In an example, a cross-section of a cup can have a shape whose two-dimensional parametric equation is X=cos(T) and Y=[sin(T)][sinM(T/2)]. In an example, a cup can have a teardrop-shaped perimeter. In an example, a teardrop shape of a cup perimeter can have an apex and/or vertex which is placed over the Tail of Spence. In an example, the apex of a teardrop shape of a cup can span the Tail of Spence. In an example, the perimeter of a portion of a cup which contacts the chest wall can have a teardrop shape, wherein the cup is rotated between right breast and left breast applications so that the apex and/or vertex of this perimeter is aligned with the Tail of Spence in both applications. In an example, the proximal perimeter of a bra cup can be teardrop shaped, wherein the longest axis of the teardrop shape has a vertical orientation (e.g. when the person wearing the bra is standing up).

In an example, a bra cup can have a crescent-shaped perimeter. In an example, the proximal perimeter of a bra cup can be substantially crescent shaped, wherein the longest axis of the crescent has an oblique orientation (e.g. from the upper outer breast quadrant to the lower inner breast quadrant) (e.g. when the person wearing the bra is standing up). In an example, a cross-section of a cup can have a paisley shape. In an example, portions of a cup which cover the upper inner, lower inner, and lower outer quadrants of the breast can have quarter-circle (e.g. quarter pie slice) cross-sectional perimeters. In an example, the height or width of a cup can be measured in a plane which is substantially orthogonal (and/or perpendicular) to the chest wall.

In an example, a cup can further comprise an adhesive ring which is gently adhered to the chest wall where a breast is attached to the chest wall—encompassing the Tail of Spence, the upper outer quadrant, the upper inner quadrant, the lower inner quadrant, and the lower outer quadrant of the breast. In an example, the perimeter of a cup can be coated with gentle adhesive to gently engage tissue close to the chest wall to keep light emitters as close as possible to the chest wall. This helps to image tissue which is as close to the chest wall as possible in addition to the main body of the breast.

In an example, a bra cup can be made from a soft, flexible, and elastic fabric. In an example, a cup can further comprise relatively inelastic components (e.g. wires or plastic strips) which are embedded in some portions of the cup. In an example, a structural layer can be less flexible, less elastic, and/or more rigid than other layers of a cup. In an example, portions of a cup which are closer to an oblique virtual plane which spans between the upper outer quadrant and the lower inner quadrant of the breast can be more elastic (e.g. lower Young's modulus) and/or more rigid than other portions of the cup. In an example, the flexibility, elasticity, and/or rigidity of the cup can be non-uniform.

In an example, a bra cup with optical sensors can be made from one or more materials selected from the group consisting of: Zinc Oxide Nanoparticles, one-dimensional nanomaterial, zero-dimensional nanomaterial, poly-Ethylene Naphthalate (PEN), poly-Ethylene Terephthalate (PET), a carbon nanomaterial, poly Lactic-co-Glycolic Acid (PLGA), a hydrogel, poly-Glycolic Acid (PGA), poly Methyl Meth-Acrylate (PMMA), poly-Ethlylene Terephthalate (PET), nanocrystals, Transition Metal Dichalcogenide (TMD), organic-inorganic composite/hybrid material, inorganic graphene, silk, graphene, and silicone rubber.

In an example a proximal portion of a cup can be less flexible, less elastic, and/or more rigid than a distal portion of a cup. In an example a proximal portion of a cup can be less flexible, less elastic, and/or more rigid than a distal portion of a cup so that the proximal portion resists outward expansion of the expandable chambers (and directs their expansion inward toward the breast) but the distal portion examples outward (e.g. away from the chest wall) expansion of the breast. In an example, the perimeter of a cross-section of a proximal portion of a bra cup can have an elliptical, oval, circular, and/or oblate-circular shape. In an example, the perimeter of a cross-section of a proximal portion of a bra cup can have a kidney-bean and/or banana shape. In an example, a proximal portion of a cup can comprise a band (e.g. cylinder or frustum) with a height (e.g. distance outward from chest wall) in the range of 2 cm to 10 cm. In an example, a proximal portion of a cup can have a perimeter diameter in the range of 7 cm to 15 cm. In an example, a proximal portion of a cup can span between one quarter and two-thirds of the proximal-to-distal distance of the overall cup.

In an example, a cup can have an outer structural layer which face away from the breast. In an example, an outer structural layer of a cup can restrict outward expansion of expandable chambers in the expandable layer so that expansion of these components is directly primarily inward toward the breast. In an example, a proximal portion of a bra cup can be reinforced with wires, strips, or bands to make it less flexible and more rigid. In an example, some portions of a bra cup (e.g. of a structural layer of the cup) can be selectively reinforced with wires while other portions of the cup are not. In an example, proximal and distal portions of a bra cup can be made separately and then attached (e.g. sewn or adhered) to each other. In this disclosure, distal means farther from the chest wall and proximal means closer to the chest wall, when the cup is worn on a person's breast. In an example, a distal portion of a cup can be more flexible and/or more elastic than a proximal portion of a cup.

In an example, a distal portion of a cup can have a concave shape. In an example, a distal portion of a cup can have a shape which is a section of an ellipsoid. In an example, the perimeter of a cross-section of a distal portion of a bra cup can have a teardrop shape. In an example, a distal portion of a cup can have a pre-expansion configuration (before expansion of the expandable chambers and outward expansion of a breast) and a post-expansion configuration (after expansion of the expandable chambers and outward expansion of the breast), wherein the distal portion is folded and/or undulating in the pre-expansion configuration. In an example, there can be a distal opening on a cup to allow maximum forward expansion of a breast during compression.

In an example, a distal portion of a cup can comprise a band (e.g. cylinder or frustum) with a height (e.g. distance outward from chest wall) in the range of 4 cm to 18 cm. In an example, a distal portion of a cup can have a pre-expansion configuration before expansion of expandable chambers and outward expansion of a breast and a second post-expansion configuration after expansion of the expandable chambers and outward expansion of a breast, wherein the post-expansion height (e.g. width along axis extending orthogonally outward from chest wall) of the distal portion is at least 10% greater than the pre-expansion height of the distal portion. In an example, a distal portion of a cup can have a perimeter diameter in the range of 4 cm to 18 cm. In an example, the height or width (measured in a plane which is orthogonal and/or perpendicular to the chest wall) of a distal portion of a cup can be less than the height of the proximal portion of the cup. In an example, the height or width (measured in a plane which is orthogonal and/or perpendicular to the chest wall) of a distal portion of a cup can be between 110% and 150% of the height of the proximal portion of the cup.

In an example, an expandable chamber can be a balloon or inflatable bladder. In an example, an expandable chamber can be an inflatable chamber with a flexible surface which is impermeable to air. In an example, a bra can comprise a plurality of hydraulic chambers which are expanded by being filled with a liquid. In an example, an expandable chamber can be expanded by liquid pressure. In an example, an expandable chamber can be expanded by being filled with a liquid (e.g. saline solution). In an example, expandable chambers can be expanded by electromagnetic actuators. In an example, expandable chambers can be expanded by being filled with a liquid.

In an example, a first expandable chamber can be in a first half of a cup which is on a first side of a virtual plane which intersects the cup and no closer than ½ inch from the plane. In an example, a first expandable chamber can be in a first (e.g. right) half of a cup which is on a first side of a vertical virtual plane which intersects the cup and a second expandable chamber can be in a second (e.g. left) half of a cup which is on a second side of the plane. In an example, a first part of an expandable layer which is on a first side of an oblique virtual plane can have at least one expandable chamber in each of: a portion of the expandable layer over the upper outer quadrant of a breast; a portion of the expandable layer over the lower outer quadrant of the breast; and a portion of the expandable layer over the lower inner quadrant of the breast.

In an example, a first part of an expandable layer on a first side of a virtual plane can have more expandable chambers than a second part of the expandable layer on a second (e.g. opposite) side of the virtual plane. In an example, a first set of expandable chambers and also light emitters can be located on one side of a virtual plane and a second set of expandable chambers and also light receivers can be located on the other side of the virtual plane. In an example, a virtual plane which separates two sets of expandable chambers can be an oblique virtual plane which is neither horizontal (e.g. axial) nor vertical (e.g. sagittal). In an example, an expandable chamber can be in a half of a cup which is on a side of a virtual plane and no closer than 1 inch from the plane. In an example, expandable chambers can be between 1 cm and 10 cm away from a virtual plane which passes through a bra cup.

In an example, expandable chambers on one side of a virtual plane can be symmetric with respect to expandable chambers on the other side of the virtual plane. In an example, first and second expandable chambers can be located to the left and to the right, respectively, of a vertical anterior-to-posterior virtual plane which intersects the cup. In an example, one part of an expandable layer which is on one side of the virtual plane can have a plurality of expandable chambers. In an example, part of an expandable layer which is on a side of an oblique virtual plane can comprise: at least one expandable chamber on a portion of the expandable layer over the upper outer quadrant of a breast; at least two expandable chambers on a portion of the expandable layer over the upper inner quadrant of the breast; and at least one expandable chamber on the portion of the expandable layer over the lower inner quadrant of the breast.

In an example, there can be a set of one or more expandable chambers on only one side of a virtual plane through a bra cup. In an example, there can be a set of one or more expandable chambers on only one side (e.g. upper outer or inner lower) of an oblique plane through a bra cup. In an example, there can be a single expandable chamber on each side of an oblique plane through a bra cup. In an example, there can be an arcuate sequence and/or series of expandable chambers in a cup on each side of a virtual plane. In an example, there can be multiple expandable chambers on each side of an oblique plane through a bra cup.

In an example, there can be multiple expandable chambers on each side of a virtual plane through a bra cup, wherein a second expandable chamber is closer to the center of the bra cup than a first expandable chamber, and wherein the second expandable chamber is larger than the first expandable chamber. In an example, there can be three expandable chambers on each side of a vertical plane through a bra cup. In an example, there can be three expandable chambers on each side of an oblique plane through a bra cup, wherein the oblique plane spans from an upper outer quadrant of a breast to a lower inner quadrant of a breast.

In an example a virtual plane which passes through a bra cup and separates sets of expandable chambers can be oriented to span between the upper outer quadrant (and the Auxiliary Tail of Space) to the lower inner quadrant so that light emitters can be as close as possible to the chest wall near the upper outer quadrant when the breast is compressed. This is desirable for optical identification of abnormal breast tissue because breast cancer is most prevalent in this quadrant. In an example, a virtual plane which is between two sets of expandable chambers can be substantially orthogonal and/or perpendicular to a person's chest wall. In an example, a virtual plane which is between two sets of expandable chambers can be horizontal when a person wearing the bra is standing (or sitting) upright.

In an example, a virtual plane which passes through a bra cup and separates sets of expandable chambers can be oblique (e.g. from upper outer breast quadrant to lower inner breast quadrant), horizontal (e.g. axial), or vertical (e.g. sagittal). In an example, an oblique virtual plane can diagonally bisect the upper outer quadrant and diagonally bisect the lower inner quadrant. In an example, the virtual plane can be an oblique virtual plane which spans from the upper outer quadrant to the lower inner quadrant of the breast. In an example, this virtual plane can be vertical (e.g. spanning between outer quadrants of a breast and lower quadrants of the breast) when a person wearing the bra is standing.

In an example, there can be expandable chambers only on the side of the cup where light emitters are located. In an example a cup can comprise a first expandable chamber on (or in) the right side of the concave interior of the cup and a second expandable chamber on (or in) the left side of the concave interior of the cup. In an example, a cup can comprise a first expandable chamber on (or in) the upper half of the interior of the cup and a second expandable chamber on (or in) the lower half of the interior of the cup. In an example, the majority of the perimeters of expandable chambers can be located interior to the proximal portion of the bra cup, with the remaining perimeters of the expandable chambers located interior to the distal portion of the bra cup.

In an example, expansion of expandable chambers can compress a breast which is between them. This can enable more accurate optical scanning of breast tissue because light rays travel a shorter distance through tissue, with less scattering. In an example, expandable chambers can be expanded until a breast is sufficiently compressed that light received by light receivers reaches a target level of light transmission intensity or resolution. In an example, the amount of light passing through breast tissue between light emitters and light receivers can be monitored during expansion of the expandable chambers.

In an example, a cup can comprise a sequence and/or series of expandable chambers. In an example, an expandable chamber can be shaped like a half of a circle, ring, or torus. In an example, an expandable chamber can have a crescent or banana shape. In an example, an expandable chamber can have a keystone or trapezoidal shape. In an example, an expandable chamber can have a toroidal or doughnut shape. In an example, an expandable chamber can have with undulating-width sections along its length. In an example, expandable chambers can have keystone and/or trapezoidal shapes when they are expanded. In an example, there can be a single crescent or banana shaped expandable chamber on each side of a virtual plane through a bra cup. In an example, there can be a single crescent or banana shaped expandable chamber on each side of an oblique plane through a bra cup, wherein the oblique plane spans from an upper outer quadrant of a bra cup to a lower inner quadrant of a bra cup.

In an example, a set of one or more expandable chambers on one side of a virtual plane through a bra cup can comprise three expandable chambers, wherein a second chamber is between first and third chambers, and wherein the second chamber is more globular in shape than the first and third chambers. In an example, there can be different numbers, sizes, shapes, and/or elasticities of expandable chambers in different quadrants of a cup. In an example, a plurality of expandable chambers can span between 50% and 75% of the perimeter of a cup. In an example, a plurality of expandable chambers can span between 60% and 85% of the interior concavity of a cup.

In an example, an expandable chamber between two other expandable chambers on a side of a virtual plane can be larger (and/or expanded more) than the two other expandable chambers on the same side. This can help to gently compress the wider central portion of a breast more than peripheral portions of the breast. This helps to flatten the breast for more accurate optical scanning. In an example, expandable chambers and/or sections which are closer to the center of a cup can be expanded to a greater extent than components and/or sections which are farther from the center of the cup. This can compress wider portions of the breast more than narrower portions of the breast, thereby reducing variation in the transmission distances of light traveling through breast tissue from light emitters to light receivers. In an example, expandable chambers in the expandable layer may be expanded to different extents so as to custom fit the bra to breasts of different sizes and shapes. In an example, expandable chambers which are farther from a virtual plane can be expanded more than expandable chambers which are closer to the virtual plane.

In an example, a plurality of hydraulic chambers can be individually and selectively expanded so that they compress wider portions of the breast to a greater degree than narrower portions of the breast, enabling better (e.g. more uniform) transmission of light through the breast. In an example, an expandable layer can comprise a plurality of hydraulic chambers which can be individually and selectively expanded so that they are expanded by different degrees, by different amounts, to different sizes, and/or to different internal pressures, thereby compressing different portions of the breast by different extents.

In an example, each expandable chamber can be connected to a separate tube, thereby enabling independent (e.g. differential and/or sequential) expansion of different expandable chambers. In an example, expansion of first and second expandable chambers in a cup can be selectively and individually controlled. In an example, hydraulic chambers can be individually and selectively expanded so that they are expanded by different degrees, by different amounts, to different sizes, and/or to different internal pressures, thereby exerting different levels of pressure on different portions of the breast. In an example, the timing of expansion of two or more expandable chambers can be individually and selectively controlled. In an example, expandable chambers in a set of expandable chambers which are closer to the center of a bra cup can be expanded (e.g. inflated) to a greater internal pressure level than expandable chambers which are farther from the center of the bra cup.

In an example, areas of a cup which are closer to a virtual plane separating other layers can be more flexible, more elastic, and/or less rigid than other areas of the cup in order to allow the breast to expand along one axis (e.g. dorsal to ventral) when it is compressed along another axis (e.g. oblique). In an example, one side of an expandable chamber can be more elastic (e.g. more elastic, more flexible, and/or less rigid) than other sides of the expandable chamber. In an example, the side and/or surface of an expandable chamber which faces away from the perimeter of a cup can be more elastic than the side of the component which faces toward the perimeter of the cup. In an example, there can be multiple expandable chambers on each side of a virtual plane through a bra cup, wherein a second expandable chamber is closer to the center of the bra cup than a first expandable chamber, and wherein the second expandable chamber is more elastic than the first expandable chamber.

In an example, one side of an expandable chamber can be thinner than other sides of the expandable chamber. In an example, different expandable chambers can be expanded at different times. In an example, expandable chambers in a set of expandable chambers which are closer to the center of a bra cup can be expanded (e.g. inflated) before expandable chambers which are farther from the center of the bra cup. In an example, expandable chambers which are closer to wider portions of a breast can be expanded before expandable chambers which are farther from wider portions of the breast. In an example, the sequence of expansion of expandable chambers can be adjusted and/or programmed to achieve different post-compression tissue width outcomes.

In an example, a bra can include tubes which conduct a flowable substance (e.g. air or water) from a pump to expandable chambers, wherein these tubes are imbedded in (or attached to) in an upper (e.g. arm) strap of the bra. In an example, a smart bra can further comprise tubes which convey a flowable substance (e.g. air or liquid) from a pump into the expandable chambers. In an example, expandable chambers can be expanded by being filled with a flowable substance (e.g. a gas or liquid) delivered through a tube, and wherein the tube goes through the interiors of the expandable chambers.

In an example, all expandable chambers (on the same side of a virtual plane) can be connected to a common tube, wherein the tube goes through the interiors of the expandable chambers. In an example, the tube can have a number of holes along its length which enable fluid communication between the interiors of the expandable chambers. In an example, there can be a common flowable-substance tube which is connected to all expandable chambers in a given set of expandable chambers (e.g. all expandable chambers on the same side of a virtual plane).

In an example, there can be a separate flowable-substance tube connected to each expandable chamber, enabling each chamber to be separately and independently expanded (e.g. inflated) or contracted (e.g. deflated). In an example, a bra can include pump which pumps a flowable substance (e.g. air or water) into the one or more expandable chambers. In an example, the back strap of the bra can include a built-in air pump which pumps air into expanding components when pressed repeatedly. In an example, a pump can be separate and only connected to the smart bra when the expandable chambers need to be expanded. In an example, a smart bra can have ports and/or valves which connect internal air tubes through the body of the bra to external air tubes from a separate air pump.

In an example, a bra can further comprise a liquid pump on the back strap of the bra, wherein the liquid pump is manually operated to pump liquid into one or more expandable chambers to compress a breast and improve the fit of the bra to the contours of breasts. In an example, a pump can be manually operated by a person who presses the pump with their hand. In an example, expandable chambers can be manually expanded by manually pumping a flowable substance (e.g. air or water) into them. In an example, a pump can be automatically operated by an impellor which is rotated by an electromagnetic motor. In an example, the pump can be automatic, but expansion can be limited based on data from sensors (e.g. pressure or motion sensors).

In an example, a bra cup can include one or more pressure sensors which measure the pressure and/or force applied to a breast by expansion of expandable chambers in order to avoid uncomfortable levels of pressure and/or force. In an example, a compression-monitoring mechanism can be an optical mechanism. In an example, expansion can be automatically stopped if a maximum target pressure is reached. In an example, is expansion is automatic, then a bra can further comprise one or more pressure sensors which monitor compression pressure on the breast and regulate pressure levels to avoid undue (potentially painful) breast compression. In an example, there can be pressure sensors which are in fluid communication with one or more expandable chambers.

In an example, a light emitter can be a Light Emitting Diode (LED). In an example, a light emitter can be a Monochromatic LED (MLED). In an example, a light emitter can be a Phosphorescent OLED (PHOLED). In an example, a light emitter can be a Side-Emitting polymer Optical Fiber (SEPOF). In an example, a light emitter can be a Vertical Cavity Surface Emitting Laser (VCSEL). In an example, a light emitter can be an Organic Light Emitting Diode (OLED). In an example, light emitters can be Light Emitting Diodes (LEDs). In an example, light emitters can emit polarized light.

In an example, one or more light emitters in a cup can be Single Photon Avalanche Diodes (SPADs). In an example, one or more light emitters in a cup can be pulsatile lasers. In an example, one or more light emitters in a cup can be monochromatic LEDs. In an example, one or more light emitters in a cup can be lasers. In an example, one or more light emitters in a cup can be an Organic Light Emitting Diodes (OLEDs). In an example, one or more light emitters in a cup can be a green-light laser.

In an example, a first light emitter can emit light with a wavelength in the range of 650 to 750 nm at a first time; a second light emitter can emit light with a wavelength in the range of 750 nm to 850 nm at a second time; and a third light emitter can emit light with a wavelength in the range of 850 nm to 950 nm at a third time. In an example, a first light emitter can emit light with a wavelength in the range of 650 to 700 nm; a second light emitter can emit light with a wavelength in the range of 700 nm to 750 nm; and a third light emitter can emit light with a wavelength in the range of 750 nm to 800 nm. In an example, a first light emitter can emit light with a wavelength in the range of 600 to 900 nm at a first time; a second light emitter can emit light with a wavelength in the range of 900 nm to 1200 nm at a second time; and a third light emitter can emit light with a wavelength in the range of 1200 nm to 1500 nm at a third time.

In an example, a first light emitter can emit light with a wavelength in the range of 600 to 800 nm at a first time; a second light emitter can emit light with a wavelength in the range of 800 nm to 1000 nm at a second time; and a third light emitter can emit light with a wavelength in the range of 1000 nm to 1200 nm at a third time. In an example, a first light emitter can emit light with a wavelength in the range of 600 to 700 nm; a second light emitter can emit light with a wavelength in the range of 700 nm to 800 nm; and a third light emitter can emit light with a wavelength in the range of 800 nm to 900 nm.

In an example, a light emitter can emit light at different wavelengths at different times. In an example, a light emitter can emit light at different wavelengths over time, within the range of 600 to 1100 nm. In an example, a light emitter can emit light at a constant frequency and/or in a constant spectral range. In an example, a light emitter can emit light with a variable frequency. In an example, different light emitters in a cup can emit light with different wavelengths selected from the group consisting of: 600, 650, 660, 680, 690, 750, 775, 780, 785, 800, 808, 810, 830, 850, and 1000 nm. In an example, different light emitters in an array of light emitters can emit light at different frequencies and/or wavelengths.

In an example, light emitters can emit light in the near-infrared (NIR) portion of the light spectrum. In an example, light emitters in a cup can emit light at different wavelengths selected from the group consisting of: 600, 650, 660, 680, 690, 750, 775, 780, 785, 800, 808, 810, 830, 850, and 1000 nm. In an example, light emitters in a cup can emit near-infrared light. In an example, the wavelengths and/or frequencies of light from light emitters can be changed. In an example, the wavelengths and/or frequencies of light from light emitters can be shifted up and down in a repeated pattern. In an example, a first set of light emitters can emit light at a first intensity or amplitude level (or at a first time) and a second set of light emitters can emit light at a second intensity or amplitude level (or at a second time).

In an example, an array of light emitters can comprise an array of nested (e.g. concentric and/or coaxial) semicircles, wherein light emitters are located along the semicircles. In an example, an array of light emitters can be a radial array, wherein spokes of the array along which light emitters are located extend out radially from an apex of the bra cup. In an example, light emitters can be configured in an orthogonal matrix (e.g. quadrilateral grid or mesh). In an example, light emitters can be configured in a hub-and-spoke array. In an example, light emitters can be configured in a half-helical array. In an example, light emitters can be configured along undulating (e.g. sinusoidal) pathways. In an example, light emitters can be evenly spaced along latitudinal lines around a breast. In an example, light emitters in a cup can be arranged in (e.g. distributed along) rings. In an example, light emitters in a cup can be arranged in (e.g. distributed along) a hexagonal (e.g. honeycomb) mesh, grid, and/or matrix.

In an example, light emitters can be closer together in the upper-right quadrant of the cup on left breast, as compared to other quadrants on that cup. In an example, light emitters in the portion of a cup which covers the upper outer quadrant of the breast can be closer together than light emitters in other portions of the cup. In an example, light emitters which are farther from the chest wall can be farther apart than light emitters which are closer to the chest wall. In an example, light emitters which are closer to the apex of a cup can be farther apart than light emitters which are farther from the apex of a cup. In an example, the density of light emitters in the portion of a cup covering the upper outer quadrant of a breast can be greater (and/or the distance between light emitters can be less) than for the portion of the cup covering the lower inner quadrant of the breast.

In an example, the distances between light emitters and detectors in the portion of a cup covering the upper outer quadrant of a breast can be greater than for the portion of the cup covering the lower inner quadrant of the breast. In an example, the distances between light emitters and detectors on a cup can be less for portions of the cup which are farther from the center (and/or apex) of the cup than for portions of the cup which are closer to the center (and/or apex) of the cup. In an example, there can be a first maximum distance between light emitters and light receivers when a cup is in its first (unexpanded) configuration and a second maximum distance between light emitters and light receivers when the cup is in its second (expanded) configuration, wherein the second average distance is less than the first average distance.

In an example, an optical layer can comprise light emitters and light receivers which are attached to, or integrated into, a flexible polymer substrate. In an example, light emitters can be attached to a fabric or textile. In an example, light emitters can be located directly on the perimeters of expandable chambers. In an example, not all light emitters may be between expandable chambers and the breast.

In an example, an array of light emitters can be to one side or the other of an oblique virtual plane which separates a first set of expandable chambers from a second set of expandable chambers. In an example, in an example, there can be equal numbers of light emitters on either side of an oblique virtual plane which spans from the Tail of Spence to the lower inner quadrant of a breast. In an example, light emitters which are farther from an oblique virtual plane spanning the upper outer quadrant and the lower inner quadrant can be farther apart than light emitters which are closer to this virtual plane. In an example, there can be an array of light emitters to one side of a virtual plane which passes through a bra cup, wherein there are between 10 and 40 light emitters in the array.

In an example, a light emitter can be positioned so as to emit light along a vector which is substantially perpendicular to a breast or cup surface. In an example, angles between the focal vectors of light emitted from light emitters and the surface of a breast or cup can increase with the distance of the light emitters from the apex of the concave surface of a breast or cup. In an example, a light emitter can emit light at an angle and/or along a focal vector which varies over time. In an example, the angles at which an array of light emitters direct light into breast tissue can be changed (e.g. controlled) by an array of movable micromirrors.

In an example, a first light emitter at a first location can emit (a pulse of) light at a first time and a second light emitter at a second location can emit (a pulse of) light at a second time. In an example, a light emitter can emit light pulses. In an example, different light emitters can be activated at different times to isolate different optical pathways from light emitters to light receivers at different times. In an example, light can be emitted from light emitters in very short pulses. In an example, light emitters can emit short pulses of light. In an example, light emitters in an array of light emitters can be activated in a circumferential sequence. In an example, light emitters in an array of light emitters can be activated in a radial sequence.

In an example, a light receiver can be a photodetector. A photodetector absorbs photons and generates electrical current, thereby converting light to electricity. In an example, a light receiver can be a thin-film photoreceptor. In an example, a light receiver can be an organic photodiode. In an example, a light receiver can be made from silicon. In an example, a light receiver can be made from carbon nanotubes. In an example, a light receiver can comprise a bicontinuous interpenetrating network of donor and acceptor materials. In an example, a light receiver in an optical layer can be selected from the group consisting of: photodiode, photomultiplier, photoconductor, avalanche photodiode, organic photodiode, organic photo-detector, silicon photodiode, photon multiplier, polarization-sensitive photodetector, Charge-Coupled Device (CCD), and Silicon Photo-Multiplier (SiPM).

In an example, a light receiver can be made with polydimethylsiloxane (PDMS) or another silicone-based polymer. In an example, light receivers can be made from one or more materials selected from the group consisting of: poly-Ethylene Terephthalate (PET), a perovskite, poly-Imide (PI), polymer and non-fullerene acceptor composite/hybrid, Transition Metal Dichalcogenide (TMD), one-dimensional nanomaterial, organic material, organic-inorganic composite/hybrid material, Jaskonite, and Zinc Oxide Nanoparticles, poly-Ethylene Naphthalate (PEN), zero-dimensional nanomaterial, colloidal quantum, Molybdenum Disulfide (MD), two-dimensional nanomaterial, a multi-conjugated organic semiconductor, and inorganic graphene.

In an example, an array of light receivers can comprise an array of nested (e.g. concentric and/or coaxial) arcs, wherein light receivers are located along the arcs. In an example an array of light receivers can be evenly spaced along longitudinal lines around a breast. In an example an array of light receivers can be configured in concentric (e.g. nested) half rings. In an example an array of light receivers can be configured in a spiral array. In an example an array of light receivers can be configured in a helical array. In an example an array of light receivers can be configured along undulating (e.g. sinusoidal) rings around a breast. In an example, an array of light receivers can be arranged in radial spokes. In an example, an array of light receivers can be arranged in (e.g. distributed along) nested (e.g. concentric) rings. In an example, there can be an array of light receivers to one side of a virtual plane which passes through a bra cup, wherein there are between 5 and 20 light receivers in the array.

In an example, light receivers can be farther apart toward the apex of a cup and closer together toward the periphery of the cup. In an example, light receivers in the portion of a cup which covers the lower outer quadrant of the breast can be closer together than light receivers in other portions of the cup. In an example, light receivers which are farther from a chest wall can be farther apart than light receivers which are closer to the chest wall. In an example, light receivers which are closer to a chest wall can be farther apart than light receivers which are farther from the chest wall. In an example, the density of light receivers in the portion of a cup covering the upper outer quadrant and Tail of Spence of a breast can be greater (and/or the distance between light receivers can be less) than for the portion of the cup covering the lower inner quadrant of the breast.

In an example, light receivers can be located between expandable chambers and a person's breast. In an example, an array of light receivers can be to the right or to the left of a vertical plane which separates a first set of expandable chambers from a second set of expandable chambers. In an example, an array of light receivers can be above or below a horizontal plane which separates a first set of expandable chambers from a second set of expandable chambers. In an example, the angles at which light receivers receive light from breast tissue can be changed (e.g. controlled) by electromagnetic actuators.

In an example, a cup can comprise a plurality of optical modules, wherein each module includes at least one light emitter and at least one light receiver. In an example, an optical layer can comprise a plurality of light receivers around the chest-wall perimeter of the optical layer and a plurality of light emitters around the concave interior of the optical layer. In an example, an optical module can include one light receiver and a plurality of light emitters. In an example, an optical module can include one light emitter and a plurality of light receivers. In an example, an optical module can include one light emitter and a plurality of light receivers distributed around the light emitter. In an example, light emitters and light receivers can be attached to, or integrated into, an elastomeric polymer layer.

In an example, a first part of a cup on a first side of a virtual plane can comprise light emitters and a second part of the cup on a second (e.g. opposite) side of the virtual plane can comprise light receivers. In an example, light emitted from the light emitters on a first side of a virtual plane can be received by light receivers on the first side of the virtual plane. In an example, there can be light emitters to a first side of the virtual plane and light receivers to a second side of the virtual plane. In an example, a cup can further comprise optical shielding and/or barriers between light emitters and light receivers on the same side of a virtual plane to reduce the direct transmission of light from light emitters to light receivers without having been reflected by, or transmitted through, breast tissue. In an example, a layer of light emitters and receivers can span the entire perimeter of a cup. In an example, a layer of light emitters and receivers can span between 50% and 75% of the perimeter of a cup.

In an example, a bra cup can include a flexible component between expandable chambers and light receivers. In an example, a bra cup can include a rigid component between expandable chambers and light emitters. In an example, an inner component (e.g. an inner ring or layer) and arrays of light emitters and receivers can surround an elliptical (or oval) shaped inner space which is configured to receive a breast in an uncompressed state. In an example, portions of an inner component (e.g. inner ring or band) which are closer to the virtual plane can be undulating and/or pleated. These undulating and/or pleated portions are more flexible and less rigid than the other portions of the inner component. This enables the inner component (e.g. inner ring or band) to compress the breast more effectively, without distorting the locations and orientations of light emitters and receivers relative to the breast.

In an example, there can be a concave component between expandable chambers and a breast, wherein light receivers are attached to this component. In an example, there can be a concave component between expandable chambers and a breast, wherein light emitters are attached to this component. In an example, there can be an arcuate layer of material between expandable chambers and light receivers. In an example, there can be an arcuate layer of material between expandable chambers and light emitters, wherein this is on one side of the virtual plane and spans across multiple expandable chambers.

In an example, a cup can further comprise undulating (e.g. sinusoidal or zigzag) electroconductive pathways which are in electrical communication with light emitters and light receivers. In an example, a cup can have a plurality of elastic electroconductive pathways which are configured in an undulating (e.g. sinusoidal) pattern. In an example, a cup can have a plurality of elastic electroconductive pathways which are configured in a spiral pattern. In an example, a cup can have a plurality of elastic electroconductive pathways which are configured in a hub-and-spoke pattern. In an example, an optical layer can comprise light emitters, light receivers, and undulating microwires which are attached to, or integrated into, an elastomeric polymer layer.

In an example, electroconductive pathways in a cup can be made with a flexible and/or elastomeric material. In an example, electroconductive pathways in a cup can be made with polyethylene terephthalate (PET). In an example, electroconductive pathways which provide power to light emitters and detectors can be embroidered onto fabric. In an example, flexible electroconductive pathways can be made from an elastomeric polymer (such as PDMS) which has been impregnated with conductive metal particles. In an example, light emitters can receive electrical power through electroconductive channels in an elastomeric polymer. In an example, light emitters can receive power from undulating (e.g. sinusoidal) wires.

In an example, a cup can comprise a plurality optical fibers (e.g. fibers, tubes, channels, or threads) which emit light from light emitters located elsewhere. In an example, a cup can further comprise optical fibers which guide light to light-exiting points which are distributed around the interior concavity of a cup. In an example, light emitters in a cup can be part of yarns or fibers which are woven to make a fabric or textile used to make the cup.

In an example, a cup can comprise an air-gap-reducing layer, wherein a second part of the layer spans the lower inner quadrant, the lower outer quadrant, the upper outer quadrant, and the Tail of Spence of a breast. In an example, a first part of an air-gap-reducing layer on a first side of a virtual plane can be pushed closer to a second part of an air-gap-reducing layer on a second (e.g. opposite) side of the virtual plane when the expandable layer is expanded and the cup is changed from its first (unexpanded) configuration to its second (expanded) configuration, thereby compressing the breast between the first and second parts. In an example, an air-gap-reducing layer can be sufficiently soft, compressible, elastomeric, and/or flexible to conform to the shape of the breast, thereby reducing air gaps between a cup and the breast. In an example, an air-gap-reducing layer of a cup can be between 0.5 mm and 2 mm thick.

In an example, an air-gap-reducing layer of a cup can be made from an alginate. In an example, an air-gap-reducing layer of a cup can be made from a starch. In an example, an air-gap-reducing layer of a cup can be made from a hydrogel. In an example, an air-gap-reducing layer of a cup can be made from collagen. In an example, an air-gap-reducing layer of a cup can be made from xanthan gum. In an example, an air-gap-reducing layer of a cup can be made from polyvinyl Chloride (PVC). In an example, an air-gap-reducing layer of a cup can be made from polymethyl Methacrylate (PMMA). In an example, an air-gap-reducing layer of a cup can be made from an interpenetrating polymer gel. In an example, an air-gap-reducing layer of a cup can be made from poly-di-methyl-siloxane (PDMS). In an example, an air-gap-reducing layer of a cup can be made from polyacrylic Acid (PA).

In an example, an air-gap-reducing layer of a cup can comprise polyethylene terephthalate (PET). In an example, an air-gap-reducing layer of a cup can have optical characteristics like those of breast tissue. In an example, an air-gap-reducing layer of a cup can have an optical scattering coefficient with a value within plus or minus 20% of the average value for normal breast tissue. In an example, an air-gap-reducing layer of a cup can have an optical absorption coefficient with a value within plus or minus 20% of the average value for normal breast tissue. In an example, an air-gap-reducing layer of a cup can have an anisotropy factor with a value like that of breast tissue. In an example, an air-gap-reducing layer of a cup can have a Shore durometer level between 5 and 30.

In an example, optical components can be separated from the surface of a breast by an air-gap-reducing layer which transmits light, but protects the optical components when a bra is washed. In an example, the air-gap-reducing layer of a cup can have the same values for one or more optical parameters as normal breast tissue. In an example, the perimeter of a first part of an air-gap-reducing layer on one side of a virtual plane can have a half-ring or horseshoe shape. In an example, the thickness, elasticity, and/or compressibility of an air-gap-reducing layer can be adjusted by changing the pressure level of fluid (or gel) in the layer. In an example, two parts of the air-gap-reducing layer on opposite sides of the virtual plane can be closer to parallel when a cup is in a second configuration than when the cup is in a first configuration.

In an example, a bra can include a data transmitter which transmits data from the light receivers to a different wearable device (e.g. a wrist-worn device), wherein this data is analyzed in the different wearable device to detect abnormal breast tissue. In an example, a smart bra can further comprise other components selected from the group consisting of: power source, data processor, wireless data transmitter, wireless data receiver, air pump, and liquid pump. In an example, a bra can include a compartment located on the back strap of the bra, wherein this compartment houses one or more of the following components: energy source (e.g. battery), data processor, data transmitter, data receiver, air pump, fluid pump, tubes, and user interface buttons. In an example, one or more other components can be located on the back strap of a bra.

In an example, a smart bra can include a data processor which controls light emitters and light receivers on the bra. In an example, a bra can include a data processing unit which analyzes data from the light receivers to detect abnormal breast tissue. In an example, a smart bra can further comprise a local data processor which is physically integrated into the smart bra. In an example, a smart bra can further comprise a local data processor which is an integral part of a smart bra. In an example, this analysis can be done in a local data processor which is part of a smart bra.

In an example, a remote data processor can be in another wearable device (e.g. a smart watch), a mobile device (e.g. a cell phone), or a remote server with which a smart bra is in wireless communication. In an example, a system can comprise a smart bra which is in wireless communication with a cell phone, smart watch, smart glasses, tablet computer, laptop computer, or remote server. In an example, data from light receivers can be wirelessly transmitted to a separate and/or remote data processor to identify and/or image abnormal breast tissue. In an example, a bra can have power source which is a battery. In an example, a bra can include an energy transducer which generates electricity from body motion. In an example, a power source which powers light emitters and other components can be an integral part of a smart bra.

In an example, a bra insert can be inserted into a right-side cup of a smart bra in a first orientation and inserted into a left-side cup of a smart bra in a second (e.g. reflected and/or rotated) orientation. In an example, a device with optical sensors to detect abnormal breast tissue can be an adhesive patch, sticker, or bandage. In an example, a device with optical sensors to detect abnormal breast tissue can be configured to be worn between a bra cup and a breast. In an example, a device with optical sensors to detect abnormal breast tissue can be a bra insert. In an example, a device with optical sensors to detect abnormal breast tissue can be removably inserted into a pocket, pouch, or other opening in a cup on a bra.

In an example, a device with optical sensors to detect abnormal breast tissue can be removably-attached to the concave interior of a bra cup (e.g. by hook-and-loop material) so that it is held in place for optical scanning, but can be removed for washing the bra without exposing optical sensors (or other electronics) to water and soap. In an example, a device with optical sensors to detect abnormal breast tissue can be inserted into a bra cup. In an example, a device with optical sensors to detect abnormal breast tissue can be modular. In an example, a system can comprise a specialized bra with one or more pockets in cups into which a concave insert with optical sensors is removably inserted. In an example, this device (e.g. cup) can be a modular device (e.g. bra insert) which is inserted between the cup of conventional bra and a breast.

In an example, a bra and/or cup can further comprise one or more marks, indicators, or sensors which help a person to place it on the same location on a breast and/or in the same orientation at different times during repeated uses (e.g. when it is worn at different times). In an example, a bra and/or cup can have one or more marks which align with specific locations on a breast, thereby achieving a desired location and/or orientation relative to a breast.

In an example, a cup or cup insert can be rotated so that light emitters and light receivers are on either side of an oblique virtual plane which spans between the upper outer quadrant of the breast and the lower inner quadrant of the breast. In an example, a cup or cup insert can be rotated for scanning from a different angle so that the same light emitters and light receivers are on either side of a horizontal plane, with the outer and inner upper quadrants on one side of the plane and the outer and lower quadrants on the other side of the plane. In an example, an optical layer of a cup can be rotated relative to a structural layer of a cup in order to change the quadrants of the breast which are most compressed and/or optically scanned. In an example, one or more layers of a cup or cup insert can be rotated to optically analyze the breast from different angles and/or provide more in-depth analysis of different breast quadrants. In an example, one or more portions of a cup or cup insert can be rotated relative to other portions of a bra in order to change the quadrants of the breast which are most compressed and/or optically scanned.

In an example, data from light receivers in a smart bra can be analyzed to identify and/or image abnormal breast tissue. In an example, changes in light caused by transmission of the light through breast tissue can be analyzed to image breast tissue and/or detect breast cancer. In an example, changes in the direction of light caused by its transmission through breast tissue can be analyzed to create an image of the breast tissue. In an example, changes in the intensity (e.g. amplitude) of light emitted from light emitters and received by light receivers which are caused by transmission of the light through breast tissue can be analyzed to detect abnormal breast tissue. In an example, changes in the intensity of light caused by its transmission through breast tissue can be analyzed to create an image of the breast tissue.

In an example, changes in the direction of light caused by its reflection from breast tissue can be analyzed to identify the levels, concentrations, and/or locations of specific biological substances (e.g. markers associated with abnormal tissue) in the breast tissue. In an example, changes in the intensity of light caused by its reflection from breast tissue can be analyzed to create an image of the breast tissue. In an example, both light transmitted through breast tissue and light reflected from breast tissue can be analyzed to detect and/or image abnormal tissue.

In an example, a smart bra can detect abnormal tissue via Frequency Domain (FD) optical analysis. In an example, a smart bra can use Diffuse Optical Imaging (DOI) to analyze the molecular composition of breast tissue, detect abnormal breast tissue, evaluate the size and shape of abnormal breast tissue, identify selected biometric parameters in breast tissue, identify the location of abnormal breast tissue, and/or image breast tissue. In an example, light which has been transmitted through and/or reflected from breast tissue and received by light receivers can be analyzed to detect and/or image abnormal breast tissue using one or more methods selected from the group consisting of: Time Reversal Optical Tomography (TROT), changes in the frequency spectrum of light transmitted through a breast, Diffuse Optical Imaging (DOI), Diffuse Optical Tomography (DOT), spectroscopic analysis, analysis of absorption and/or scattering of light transmitted through a breast, Near-Infrared spectroscopy (NIRS), functional Near-Infrared spectroscopy (fNIRS), changes in the intensity or amplitude of light transmitted through a breast, changes in the phase of light transmitted through a breast, Diffuse Correlation spectroscopy (DCS), Carlavian Curve analysis (CCA), machine learning, neural network analysis, broadband spectroscopy, and/or changes in the spectral distribution of light transmitted through a breast.

In an example, changes in amplitude and/or spectrum of light from the light emitters which are caused by transmission of the light through breast tissue can be analyzed to image breast tissue and/or detect breast cancer. In an example, changes in the intensity and/or spectral distribution of light caused by transmission through breast tissue can be analyzed to create a (3D) image which shows variation in breast tissue composition. In an example, changes in the intensity and/or spectral distribution of light caused by transmission through breast tissue can be analyzed to create a (3D) image of breast tissue. In an example, changes in the spectrum (e.g. spectral distribution) of light emitted from light emitters and received by light receivers which are caused by transmission of the light through breast tissue can be analyzed to evaluate the molecular composition of breast tissue and detect abnormal breast tissue. In an example, changes in the spectrum of light caused by its transmission through breast tissue can be analyzed to create an image of the breast tissue.

In an example, spectroscopic analysis of near-infrared light which has been transmitted through and/or reflected from breast tissue can be done to identify the levels, concentrations, and/or locations of specific biological substances (e.g. markers associated with abnormal tissue) in the breast tissue. In an example, spectroscopic analysis of light which has been transmitted through and/or reflected from breast tissue can be done to identify the sizes, configurations, and/or locations of specific biological structures associated with abnormal tissue. In an example, spectroscopic analysis of light which has been reflected from breast tissue can be done to identify the sizes, configurations, and/or locations of specific biological structures associated with abnormal tissue.

In an example, light which has been transmitted through and/or reflected from breast tissue can be analyzed to detect and/or image the locations, sizes, and/or configurations of extracellular matrix structures in breast tissue. In an example, light which has been transmitted through and/or reflected from breast tissue can be analyzed to detect and/or image levels, concentrations, and/or locations of deoxyhemoglobin in breast tissue. In an example, light which has been transmitted through and/or reflected from breast tissue can be analyzed to detect and/or image levels, concentrations, and/or locations of collagen in breast tissue. In an example, light which has been transmitted through and/or reflected from breast tissue can be analyzed to detect and/or image levels, concentrations, and/or locations of oxygen in breast tissue.

In an example, collecting data on changes in transmitted light from a large number of different light pathways (e.g. light emitter and light receiver combinations) enables more accurate detection of abnormal tissue and identification of tissue attributes (such as location, size, shape, and composition). In an example, data patterns concerning light changes in multiple pathways can be analyzed by machine learning and/or artificial intelligence to detect abnormal tissue and identify tissue attributes. In an example, a smart bra can be worn for a short period of time on a periodic basis (e.g. annually, monthly, weekly, or daily) in order to obtain a periodic longitudinal time series of optical of breast tissue for identification of changes in tissue composition. In an example, results from more recent breast scans using a smart bra can be compared and/or contrasted with earlier scans to help detect growth of abnormal breast tissue.

In an example, a device can be a bra. In an example, a device can be embodied in a smart bra which includes optical sensors and electronic components. In an example, a bra can have two cups with optical sensors and expandable chambers so that data concerning both breasts can be compared and contrasted to better detect abnormal tissue in one of the breasts. In an example, a cup on one side of a smart bra can have a similar (e.g. same, but reflected across a central vertical plane) configuration of expanding components, light emitters, and light receivers as a cup on the other side of the smart bra. In an example, a left-side version of a bra cup can be symmetric to a right-side version of the cup.

In an example, a smart bra can be custom-fitted to a specific breast. In an example, this device can be embodied in a smart bra which can be custom fitted to a particular breast size and shape by selective expansion of expandable chambers in an expandable layer in a bra cup. In an example, a cup can have a concave shape which fits over a breast. In an example, a bra cup can have a conical, partial-conical, and/or frustal shape. In an example, a bra cup can have a semi-ellipsoidal shape. In an example, a cross-section of a cup can have an elliptical or oval shape. In an example, the proximal perimeter of a bra cup can be substantially elliptical and/or oval, wherein the longest axis of the ellipse and/or oval has a vertical orientation (e.g. when the person wearing the bra is standing up).

In an example, a bra cup can have a teardrop shape. In an example, a cross-section of a cup can have a teardrop cross-sectional shape. In an example, a cross-section of a cup can have a paisley shape. In an example, a cup can have a teardrop-shaped perimeter. In an example, a teardrop-shaped cross-section of a cup can have a shape whose two-dimensional parametric equation is X=cos(T) and Y=[sin(T)][sinM(T/2)]. In an example, the perimeter of a cup which is closest to the chest wall can have a teardrop shape. A teardrop shape can be good for encompassing the Tail of Spence and fully spanning where the upper outer quadrant connects to the chest wall. In an example, the point of a teardrop-shaped cup can cover the Axillary Tail of Spence. In an example, the proximal perimeter of a bra cup can be teardrop shaped, wherein the longest axis of the teardrop shape has an oblique orientation (e.g. from the upper outer breast quadrant to the lower inner breast quadrant) (e.g. when the person wearing the bra is standing up). In an example, the proximal perimeter of a bra cup can be substantially crescent shaped. In an example, the bra cup has a kidney-bean and/or banana shape. In an example, the diameter of a cup can be measured in a plane which is substantially parallel to the chest wall.

In an example, a cup can further comprise disposable adhesive strips (or rings) which are removably attached to the inner layer of the cup and to breast tissue near the chest wall in order to gently engage tissue close to the chest wall. This enables light emitters to be as close as possible to the chest wall. In an example, the perimeter of a cup which is closest to the chest wall can be gently adhered to the chest wall. In an example, a bra cup can be flexible and/or elastic. In an example, a central portion of a cup can be less elastic (e.g. higher Young's modulus) and/or more rigid than other portions of the cup. In an example, a portion of a cup which is closer to the apex of a cup can be less elastic and/or more rigid than other portions of the cup. In an example, perimeter portions of a cup can be less elastic and/or more rigid. In an example, the base of a cup which is closest to the chest wall can be less flexible, less elastic, and/or more rigid than the rest of the cup. In an example, the perimeter of a cup which is closest to a chest wall can be less elastic (e.g. higher Young's modulus) and/or more rigid than other portions of the cup.

In an example, a bra cup can be made from a fabric and/or a textile. In an example, a bra cup with optical sensors can be made from one or more materials selected from the group consisting of: poly-Imide (PI), parylene, a perovskite, poly-Lactic Acid (PLA), acrylic, polymer and non-fullerene acceptor composite/hybrid, black phosphorus, poly-Styrene (PS), poly-Ethylene Glycol Di-Acrylate (PEGDA), cellulose, poly-Urethane (PU), chitosan, quantum dots, colloidal quantum, shape Memory Photonic Crystal Fiber (SMPF), and organic material.

In an example a proximal portion of a cup can be less flexible, less elastic, and/or more rigid than a distal portion of a cup so that it resists outward expansion of the expandable chambers and directs their expansion inward toward the breast. In an example, a proximal portion of a cup can comprise a relatively inflexible and/or inelastic band. In an example, a proximal portion of a cup can have a narrow cylindrical shape. In an example, the perimeter of a cross-section of a proximal portion of a bra cup can have a teardrop shape. In an example, the proximal perimeter of this proximal portion can have a circular, elliptical, oval, teardrop, or crescent shape. In an example, a proximal portion of a cup can comprise a band (e.g. cylinder or frustum) with a perimeter diameter in the range of 5 cm to 20 cm. In an example, a proximal portion of a cup can comprise a band (e.g. cylinder or frustum) with a height (e.g. distance outward from chest wall) in the range of 3 cm to 8 cm. In an example, a proximal portion of a cup can have a height (e.g. distance outward from chest wall) in the range of 2 cm to 10 cm.

In an example, a cup for optical scanning of breast tissue can comprise three layers: an inner optical layer, an expandable layer, and an outer structural layer. In an example, an outer structural layer of a cup can restrict outward expansion of expandable chambers in the expandable layer so that expansion of these components is directly primarily inward to compress the breast. In an example, a proximal portion of a bra cup can comprise fabric or a textile with imbedded (or attached) wires. In an example, an outer structural layer of a bra cup can be reinforced with wires so that pressure from expansion of an inner expandable layer is directed primarily inward toward a breast to compress the breast for better optical scanning. In an example, some portions of a bra cup can be reinforced by wires. In an example, proximal and distal portions of a bra cup can be made from a single (e.g. continuous) piece of material and then separately modified to change their respective levels of flexibility and/or elasticity. In an example, a distal portion of a cup can be more flexible and/or elastic than the proximal portion of the cup. This allows forward and/or outward expansion of a breast when expandable chambers are expanded and a breast is gently compressed.

In an example, a distal portion of a cup can have a shape which is a section of a sphere. In an example, a perimeter of a cross-section of a distal portion of a bra cup can have an elliptical, oval, circular, and/or oblate-circular shape. In an example, the perimeter of a cross-section of a distal portion of a bra cup can have a kidney-bean and/or banana shape. In an example, a distal portion of a cup can have a pre-expansion configuration before expansion of expandable chambers and outward expansion of a breast and a second post-expansion configuration after expansion of the expandable chambers and outward expansion of a breast, wherein the post-expansion configuration is between 10% and 50% greater than the pre-expansion configuration. In an example, a distal portion of a cup can have a pre-expansion configuration before expansion of expandable chambers and outward expansion of a breast and a second post-expansion configuration after expansion of the expandable chambers and outward expansion of a breast, wherein the post-expansion height (e.g. width along axis extending orthogonally outward from chest wall) of the distal portion is between 10% and 50% greater than the pre-expansion height of the distal portion. In an example, a distal portion of a cup can have a height (e.g. distance outward from chest wall) in the range of 2 cm to 10 cm. In an example, the height or width (measured in a plane which is orthogonal and/or perpendicular to the chest wall) of a distal portion of a cup can be greater than the height of the proximal portion of the cup.

In an example, an expandable chamber can be a liquid-filled chamber or bladder with a flexible surface which is impermeable to liquid. In an example, expandable chambers can be balloons. In an example, a cup can comprise a plurality of chambers which are expanded by being filled with a gas (e.g. air). In an example, an expandable chamber can be expanded by inflation with a gas (e.g. air). In an example, an expandable chamber can be expanded by a pneumatic mechanism. In an example, expandable chambers can be expanded by being filled with a flowable substance such as a gas (e.g. air) or liquid (e.g. water). In an example, expandable chambers can be filled with a flowable substance (e.g. air or some other gas).

In an example, a bra for optical detection of abnormal breast tissue can comprise an expandable layer with a plurality of expandable chambers, wherein a first part of the expandable layer is on the first side of the virtual plane and a second part of the expandable layer is on the second side of the virtual plane. In an example, a first expandable chamber can be in a first half of a cup which is on a first side of a virtual plane which intersects the cup and no closer than 1 inch from the plane. In an example, a first expandable chamber can be in a first (e.g. lower-left) half of a cup which is on a first side of an oblique virtual plane which intersects the cup and a second expandable chamber can be in a second (e.g. upper-right) half of a cup which is on a second side of the plane.

In an example, a first part of an expandable layer which is on a first side of an oblique virtual plane can comprise: at least one expandable chamber on a portion of the expandable layer over the upper outer quadrant of a breast; at least two expandable chambers on a portion of the expandable layer over the lower outer quadrant of the breast; and at least one expandable chamber on the portion of the expandable layer over the lower inner quadrant of the breast. In an example, a first set of expandable chambers can be on one side of a virtual plane which passes through a bra cup and a second set of expandable chambers can be on the other side of this virtual plane. In an example, a set of one or more expandable chambers on one side of a virtual plane through a bra cup can comprise three expandable chambers, wherein a second chamber is between first and third chambers, and wherein the second chamber is larger than the first and third chambers.

In an example, an expandable chamber can be in a half of a cup which is on a side of a virtual plane and no closer than ¼ inch from the plane. In an example, expandable chambers can be asymmetric with respect to reflection across a virtual plane. In an example, expandable chambers can be substantially symmetric with respect to reflection across a virtual plane. In an example, expandable chambers which are farther from the virtual plane can be expanded more than expandable chambers which are closer to the virtual plane. In an example, first and second expandable chambers can be located below and above, respectively, a horizontal anterior-to-posterior virtual plane which intersects the cup. In an example, part of an expandable layer which is on a side of the oblique virtual plane can have more expandable chambers over the upper inner quadrant of the breast than over the lower inner quadrant of the breast.

In an example, there can be a plurality of expandable chambers on each side of a virtual plane which passes through a cup. In an example, there can be a set of one or more expandable chambers on only one side (e.g. right or left) of a vertical plane through a bra cup. In an example, there can be a single expandable chamber on each side of a virtual plane through a bra cup. In an example, there can be a single expandable chamber on each side of an oblique plane through a bra cup, wherein the oblique plane spans from an upper outer quadrant of a bra cup to a lower inner quadrant of a bra cup. In an example, there can be multiple expandable chambers on each side of a virtual plane through a bra cup. In an example, there can be multiple expandable chambers on each side of an oblique plane through a bra cup, wherein the oblique plane spans from an upper outer quadrant of a bra cup to a lower inner quadrant of a bra cup.

In an example, there can be multiple expandable chambers on each side of a virtual plane through a bra cup, wherein a second expandable chamber is closer to the center of the bra cup than a first expandable chamber, and wherein the second expandable chamber has a thicker perimeter layer than the first expandable chamber. In an example, there can be three expandable chambers on each side of an oblique plane through a bra cup. In an example, this cup can be oriented around a virtual plane, with one set of optical and expandable chambers on one side of this virtual plan and a second set of optical and expandable chambers on the opposite side of this virtual.

In an example, a virtual plane can be oblique—spanning from the upper outer quadrant of the breast to the lower inner quadrant of the breast. This oblique configuration for the virtual plane can enable the cup to more accurately detecting abnormal tissue in the upper outer quadrant of the breast and also the associated Tail of Spence. These areas are disproportionately prone to malignancy as compared to other areas of the breast. In an example, a virtual plane which is between two sets of expandable chambers can be oblique, extending from the upper-outer quadrant of a breast to the lower-inner quadrant of the breast. In an example, a virtual plane which passes through a cup can intersect the upper inner quadrant and the lower outer quadrant of a breast. In an example, an oblique virtual plane can bisect the upper outer quadrant and bisect the lower inner quadrant. In an example, an oblique virtual plane can intersect the upper outer quadrant (and/or the Tail of Spence) and the lower inner quadrant of a breast. In an example, this virtual plan can be an oblique virtual plane. In another example, a virtual plane can be oblique (e.g. spanning from an upper outer quadrant of a breast to a lower inner quadrant of a breast). A design with an oblique virtual plane can be better for detecting abnormal tissue in the upper outer quadrant of a breast and the Tail of Spence. These areas are more prone to develop malignancy than other areas of the breast or chest wall.

In an example, there can be expandable chambers only on the side of the cup where light detectors are located. In an example, a cup can comprise a first expandable chamber on (or in) the upper right quadrant (from a frontal corona view) of the cup for a right-side breast and a second expandable chamber on (or in) the lower left quadrant (from a frontal corona view) of the interior of the cup. In an example, expandable chambers can be located interior to the proximal portion of a bra cup, but not the distal portion of the bra cup. In an example, a portion of a breast between two expandable chambers can have a first width when a cup is in a first (unexpanded) configuration and a second width when the cup is in a second (expanded) configuration, wherein the second width is less than the first width. In an example, gentle and partial outward flattening of the breast by this smart bra is less than flattening caused by traditional mammography, but still sufficient to enable greater light transmission through the breast (e.g. with less scattering).

In an example, expandable chambers can be expanded until either a target level of light transmission is achieved or the person wearing the bra indicates discomfort. In an example, expansion of expandable chambers can be adjusted to try to achieve breast compression without undue pressure. In an example, the expandable chambers are expanded until the amount of light scattering is reduced to a target level and/or percentage or the person wearing the bra indicates discomfort.

In an example, an expandable chamber can be shaped like a section of a circle, ring, or torus. In an example, an expandable chamber can have a conic-section shape. In an example, an expandable chamber can have a disk shape in a first configuration and an ellipsoidal shape in an expanded second configuration. In an example, an expandable chamber can have a pleated and/or folded shape like an accordion or bellows. In an example, an expandable chamber can have an arcuate shape. In an example, expandable chambers can comprise an undulating (e.g. sinusoidal) sequence of connected fluid (e.g. gas or liquid) filled chambers. In an example, there can are two (parallel) series and/or sequences of expandable chambers on each side of a virtual plane. In an example, there can be a single crescent or banana shaped expandable chamber on each side of a vertical plane through a bra cup. In an example, there can be multiple expandable chambers on each side of a virtual plane through a bra cup, wherein a second expandable chamber is closer to the center of the bra cup than a first expandable chamber, and wherein the second expandable chamber is more globular in shape than the first expandable chamber.

In an example, expandable chambers in portions of a cup which cover the lower outer and upper inner quadrants of the breast can have different shapes than expandable chambers in other portions of the cup. In an example, a plurality of expandable chambers can span between 50% and 75% of the interior concavity of a cup. In an example, a plurality of expandable chambers can span all of the interior concavity of a cup. In an example, a first expandable chamber can be expanded to a different size than a second expandable chamber. In an example, an expandable chamber which is central to one side of a cup can be larger, or expanded more, than other expandable chambers on that side of the cup.

In an example, expandable chambers in a first part of an expandable layer on a first side of a virtual plane can have different sizes and/or shapes than expandable chambers in a second part of the expandable layer on a second (e.g. opposite) side of the virtual plane. In an example, expandable chambers in the expandable layer may be expanded to different extents so as to provide uniform pressure across the surface of a breast. In an example, expandable chambers which are closer to the center of a bra cup can be expanded (e.g. inflated) more (e.g. to a greater degree or percentage) than expandable chambers which are farther from the center of the bra cup.

In an example, a breast can be compressed into a flatter configuration (for better analysis by transmitted light) by individual and selectively expanding central expandable chambers and/or sections more than non-central expandable chambers and/or sections. In an example, a plurality of inflatable chambers can be individually and selectively inflated so that they compress wider portions of the breast to a greater degree than narrower portions of the breast, enabling better (e.g. more uniform) transmission of light through the breast. In an example, differential expansion of different components can help to compress a portion of a breast into a shape with more uniform width (e.g. flatter) for better scanning. In an example, expandable chambers can be individually and selectively expanded so that they are configured to compress wider portions of the breast to a greater degree than narrower portions of the breast. In an example, expansion of individual expandable chambers in a cup can be individually and selectively controlled. In an example, inflatable chambers can be individually and selectively inflated y different degrees, by different amounts, to different sizes, and/or to different internal air pressures.

In an example, expandable chambers in portions of a cup which cover the lower outer and upper inner quadrants of the breast can be more elastic and/or stretchable than expandable chambers in other portions of the cup. In an example, the side and/or surface of an expandable chamber which faces toward the center of the breast can have a lower Young's modulus than the side of the component which faces away from the center of the breast. In an example, the side and/or surface of an expandable chamber which faces toward the center of the breast can be more elastic than the side of the component which faces away from the center of the breast. In an example, there can be multiple expandable chambers on each side of a virtual plane through a bra cup, wherein a second expandable chamber is closer to the center of the bra cup than a first expandable chamber, and wherein the second expandable chamber has a more-elastic perimeter layer than the first expandable chamber.

In an example, a side of an expandable chamber which faces toward a breast can be thinner than a side of the component which faces away from the breast. In an example, expandable chambers in a set of expandable chambers which are farther from the center of a bra cup can be expanded (e.g. inflated) before expandable chambers which are closer to the center of the bra cup. In an example, expandable chambers which are closer to the center of a cup can be expanded before expandable chambers which are farther from the center of the cup.

In an example, a bra can include tubes which conduct a flowable substance (e.g. air or water) from a pump to expandable chambers, wherein these tubes are imbedded in (or attached to) in a circumferential (e.g. side) strap of the bra. In an example, expandable chambers can be expanded by being filled with a flowable substance (e.g. a gas or liquid) delivered through the tube. In an example, all expandable chambers (or at least all chambers on the same side of a virtual plane) can be connected to a common tube. In an example, there can be a common flowable-substance tube which passes through all expandable chambers. In an example, each expandable chamber can be in fluid communication with a pump through a separate fluid tube or channel between the pump and the expandable chamber.

In an example, a smart bra can further comprise an air pump or liquid pump. In an example, the pump can be permanently integrated into (the back strap of) the smart bra. In an example, a pump can be separate from a wearable bra, but connected to the bra in order to expand the expandable chambers (e.g. via one or more tubes). In an example, air tubes from the air pump can be connected to the bra via the ports and/or valves for expanding components within the cups of the bra. In an example, a bra can include an air pump on the back strap, wherein the air pump is manually operated to pump air into one or more expandable chambers to compress a breast and improve the fit of the bra to the contours of breasts. In an example, a pump can be manually operated. In an example, expandable chambers can be manually expanded by air pumped from a manually-activated air pump. This provides the person wearing the bra with direct control over the pressure exerted by the expandable layer on the breast, which can be useful for avoiding undue (potentially painful) breast compression.

In an example, expandable chambers can be automatically expanded, such as by an automatically-activated air pump. In an example, a compression-monitoring mechanism can comprise one or more pressure sensors and/or strain sensor. In an example, a smart bra can further comprise pressure sensors. In an example, expansion of expandable chambers can be controlled (e.g. limited) based on pressure levels on those chambers in order to decrease or completely avoid uncomfortable compression of a breast. In an example, optimal inflation parameters for expandable chambers can be identified for a specific breast based on optical transmission results and/or pressure results. In an example, a bra cup can include one or more motion and/or bend sensors which measure the amount (e.g. relative distance) by which expansion of expandable chambers has compressed a breast.

In an example, a light emitter can be a laser LED. In an example, a light emitter can be a Light-Emitting Electrochemical Cell (LEC). In an example, a light emitter can be a Multi-Wavelength Light Emitting Diode (MWLED). In an example, a light emitter can be a Quantum Dot LED (QLED). In an example, a light emitter can be a Single Photon Avalanche Diode (SPAD). In an example, a light emitter can be an Active Matrix Organic Light-Emitting Diode (AMOLED). In an example, a light emitter can emit coherent light. In an example, light emitters can be made by 3D printing. In an example, one or more light emitters in a cup can be tunable LEDs. In an example, one or more light emitters in a cup can be Resonant Cavity Light Emitting Diodes (RCLEDs). In an example, one or more light emitters in a cup can be near-infrared light emitters. In an example, one or more light emitters in a cup can be MicroLEDs. In an example, one or more light emitters in a cup can be infrared light emitters. In an example, one or more light emitters in a cup can be Active Matrix Organic Light-Emitting Diodes (AMOLEDs).

In an example, a first light emitter can emit light with a wavelength in the range of 650 to 700 nm; a second light emitter can emit light with a wavelength in the range of 700 nm to 750 nm; and a third light emitter can emit light with a wavelength in the range of 750 nm to 800 nm. In an example, a first light emitter can emit light with a wavelength in the range of 650 to 750 nm; a second light emitter can emit light with a wavelength in the range of 750 nm to 850 nm; and a third light emitter can emit light with a wavelength in the range of 850 nm to 950 nm. In an example, a first light emitter can emit light with a wavelength in the range of 600 to 900 nm and a second light emitter can emit light with a wavelength in the range of 900 nm to 1200 nm.

In an example, a first light emitter can emit light with a wavelength in the range of 600 to 800 nm; a second light emitter can emit light with a wavelength in the range of 800 nm to 1000 nm; and a third light emitter can emit light with a wavelength in the range of 1000 nm to 1200 nm. In an example, a first set of light emitters can emit light at a first frequency and/or wavelength (or in a first spectral range), a second set of light emitters can emit light at a second frequency and/or wavelength (or in a second spectral range), and a third set of light emitters can emit light at a third frequency and/or wavelength (or in a third spectral range). In an example, a light emitter can emit light at different wavelengths over time, within the range of 600 to 1100 nm.

In an example, a light emitter can emit light at different wavelengths over time selected from the group consisting of: 600, 650, 660, 680, 690, 750, 775, 780, 785, 800, 808, 810, 830, 850, and 1000 nm. In an example, a light emitter can emit light within a spectral range which varies over time. In an example, different light emitters can emit light at different wavelengths. In an example, different light emitters in a ring can emit light at different wavelengths. In an example, different light emitters in an array can emit light at different wavelengths. In an example, light emitters can emit near-infrared (NIR) light. In an example, light emitters in a cup can emit light at different wavelengths over time, within the range of 600 to 1100 nm. In an example, light emitters on a cup can emit frequency and/or wavelength modulated light. In an example, the wavelengths and/or frequencies of light from light emitters can be changed in a sequential manner. In an example, the wavelengths and/or frequencies of light from light emitters can be varied in a repeated pattern. In an example, a light emitter can emit intensity or amplitude-modulated light.

In an example, an array of light emitters can comprise an array of nested (e.g. concentric and/or coaxial) rings, wherein light emitters are located around the rings. In an example, light emitters can be configured in concentric (e.g. nested) rings. In an example, light emitters can be configured in a star-burst array. In an example, light emitters can be configured in a honeycomb array (e.g. hexagonal grid or mesh). In an example, light emitters can be configured in a checkerboard array. In an example, light emitters can be equally distributed on a given side (e.g. right or left, lower or upper) of a cup. In an example, light emitters in a cup can be arranged in radial spokes. In an example, light emitters in a cup can be arranged in (e.g. distributed along) nested (e.g. concentric) rings.

In an example, light emitters can be closer together in the upper-left quadrant of the cup on right breast, as compared to other quadrants on that cup. In an example, light emitters in the portion of a cup which covers the upper outer quadrant and the Tail of Spence of the breast can be closer together than light emitters in other portions of the cup. In an example, light emitters which are farther from the apex of a cup can be farther apart than light emitters which are closer to the apex of a cup. In an example, the density of light emitters on a cup can be less (and/or the distance between light emitters can be greater) for portions of the cup which are farther from the center (and/or apex) of the cup than for portions of the cup which are closer to the center (and/or apex) of the cup.

In an example, the density of light emitters in the portion of a cup covering the upper outer quadrant of a breast can be less (and/or the distance between light emitters can be greater) than for the portion of the cup covering the lower inner quadrant of the breast. In an example, the distances between light emitters and detectors in the portion of a cup covering the upper outer quadrant of a breast can be less than for the portion of the cup covering the lower inner quadrant of the breast. In an example, the distances between light emitters and detectors on a cup can be greater for portions of the cup which are farther from the center (and/or apex) of the cup than for portions of the cup which are closer to the center (and/or apex) of the cup.

In an example, at least some light emitters can be configured to be between a set of one or more expandable chambers and a breast. In an example, light emitters can be encapsulated with a waterproof coating for protection from moisture. In an example, light emitters can be located between expandable chambers and a person's breast. In an example, an array of light emitters can be on one side of a virtual plane which passes through a bra cup, wherein on one side of a virtual plane means being in space which is on one side of the virtual plane, not being located directly on the side of the virtual plane. For example, the array of light emitters can be on one side of a virtual plane, but still be centimeters away from the virtual plane. As used herein, the phrase on one side of can also be interpreted as to one side of. In an example, light emitters can all be on the same side of this oblique virtual plane. In an example, there can be a plurality of light emitters to one side of a virtual plane which passes through a bra cup.

In an example, a light emitter can be positioned so as to emit light toward a particular light receiver. In an example, angles between the focal vectors of light beams emitted from light emitters and the surface of a breast or cup can vary with the distance of the light emitters from the apex of a cup. In an example, a cup can further comprise a plurality of mirrors which change the vectors of light rays from a plurality of light emitters. In an example, an optical component can include an electromagnetic actuator which changes the angle and/or focal vector of light emission over time. In an example, the angles at which light emitters direct light into breast tissue can be changed (e.g. controlled) by electromagnetic actuators.

In an example, a first light emitter can emit a pulse of light with a first duration and a second light emitter can emit a pulse of light with a second duration, wherein the second duration is greater than the first duration. In an example, a light emitter can emit light via Alternating Current Electroluminescence (ACEL). In an example, different light emitters can be activated (e.g. pulsed) at different times (e.g. sequentially). In an example, light emission from light emitters can be multiplexed. In an example, light emitters in a cup can emit light in pulses. In an example, light emitters in an array of light emitters can be activated in a sequential manner. In an example, light emitters on a right side of a cup can emit (a pulse of) light at a first time and light emitters on the left side of a cup can emit (a pulse of) light at a second time, or vice versa.

In an example, a light receiver can be a flexible organic photodetector (OPD). In an example, a light receiver can be a photodiode. In an example, a light receiver can be an avalanche photo diode (APDs) or PIN photodiode. In an example, a light receiver can be flexible. In an example, a light receiver can be made from PEDOT:PSS. In an example, a light receiver can be made with an acrylic elastomer. In an example, a light receiver can comprise a fast-gated detector. In an example, a light receiver can be made from semiconducting polymer.

In an example, an array of light receivers can be a radial array, wherein spokes of the array along which light receivers are located extend out radially from an apex of the bra cup. In an example an array of light receivers can be evenly spaced along latitudinal lines around a breast. In an example an array of light receivers can be configured in an orthogonal matrix (e.g. quadrilateral grid or mesh). In an example an array of light receivers can be configured in a hub-and-spoke array. In an example an array of light receivers can be configured in a half-helical array. In an example an array of light receivers can be configured along undulating (e.g. sinusoidal) pathways. In an example, an array of light receivers can be arranged in a hub-and-spoke configuration. In an example, an array of light receivers can be arranged in (e.g. distributed along) an orthogonal mesh, grid, and/or matrix. In an example, there can be an array of light receivers to one side of a virtual plane which passes through a bra cup, wherein there are between 10 and 40 light receivers in the array.

In an example, light receivers in the portion of a cup which covers the upper outer quadrant of the breast can be closer together than light receivers in other portions of the cup. In an example, light receivers which are farther from the apex of a cup can be farther apart than light receivers which are closer to the apex of the cup. In an example, light receivers which are closer to the apex of a cup can be farther apart than light receivers which are farther from the apex of the cup. In an example, the density of light receivers in the portion of a cup covering the upper outer quadrant of a breast can be greater (and/or the distance between light receivers can be less) than for the portion of the cup covering the lower inner quadrant of the breast. In an example, the density of light receivers in a cup can be less (and/or the distance between light receivers can be greater) for portions of the cup which are farther from the center (and/or apex) of the cup than for portions of the optical layer which are closer to the center (and/or apex) of the cup.

In an example, at least some light receivers can be between expandable chambers and the breast. In an example, not all light receivers need be between expandable chambers and the breast. In an example, an array of light receivers can be to one side or the other of an oblique virtual plane which separates a first set of expandable chambers from a second set of expandable chambers. In an example, there can be a plurality of light receivers to one side of a virtual plane which passes through a bra cup.

In an example, a cup can comprise light emitters and light receivers which are attached to, or integrated into, an elastomeric substrate. In an example, an optical layer can comprise a plurality of light emitters around the chest-wall perimeter of the optical layer and a plurality of light receivers around the concave interior of the optical layer. In an example, an optical module can include one light receiver and a plurality of light emitters which are evenly-distributed around the light receiver. In an example, an optical module can include one light emitter and at least four light receivers which are evenly-distributed around the light emitter. In an example, both light emitters and light receives can be on the same side of a virtual plan.

In an example, a first part of a cup on a first side of a virtual plane can comprise both light emitters and light receivers. In an example, light emitters can be on one side of a virtual plane which passes through a cup and light receivers can be on the other side of the virtual plane. In an example, there can be light emitters on a first side of a virtual plane and light receivers on a second side of the virtual plane. In an example, a cup can further comprise opaque shielding and/or barriers between light emitters and light receivers on the same side of a virtual plane to reduce the direct transmission of light from light emitters to light receivers without having been reflected by, or transmitted through, breast tissue. In an example, a layer of light emitters and receivers can span between 60% and 85% of the perimeter of a cup. In an example, a layer of light emitters and receivers can span between 50% and 75% of the interior concavity of a cup.

In an example, a bra cup can also comprise an inner component (e.g. an inner ring or layer) located between light emitters or receivers and expandable chambers. This inner component (e.g. inner ring or layer) can help to keep light emitters and receivers in the proper locations and orientations relative to a breast as the expandable chambers are expanded. In an example, a bra cup can include a flexible component between expandable chambers and light emitters. In an example, an inner component (e.g. an inner ring or layer) between light emitters and expandable chambers can comprise: a first portion on a first side of the virtual plane, a second portion on a second side of the virtual plane, and two undulating and/or pleated portions which cross the virtual plane.

In an example, an inner component (e.g. inner ring or band) can have a generally elliptical, oval, circular, or oblate-circular shape before expansion of the expandable chambers and a football shape after expansion of the expandable chambers. In an example, there can be a concave component between expandable chambers and light receivers, wherein this component spans multiple expandable chambers. In an example, there can be a concave component between expandable chambers and light emitters, wherein this component spans multiple expandable chambers. In an example, there can be a layer of material between expandable chambers and light receivers. In an example, there can be an arcuate layer of material between expandable chambers and light receivers, wherein this layer spans multiple expandable chambers. In an example, there can be an arcuate layer of material between expandable chambers and light emitters.

In an example, a cup can also comprise electroconductive pathways which are connected to light emitters and light receivers. In an example, a cup can further comprise undulating (e.g. sinusoidal or zigzag) wires which are in electrical communication with light emitters and light receivers. In an example, a cup can have a plurality of elastic electroconductive pathways which are configured in an orthogonal matrix (e.g. quadrilateral grid or mesh). In an example, a cup can have a plurality of elastic electroconductive pathways which are configured in a row-and-column pattern.

In an example, a cup can have a plurality of elastic electroconductive pathways which are configured in a honeycomb (e.g. hexagonal) grid or mesh. In an example, an optical layer can comprise light emitters, light receivers, and electroconductive pathways which are attached to, or integrated into, an elastomeric polymer layer. In an example, electroconductive pathways in a cup can be made with a combination of poly(3,4-ethylenedioxythiophene) polystyrene sulfonate (PEDOT:PSS) and carbon nanotubes. In an example, electroconductive pathways in a cup can be made with polydimethylsiloxane (PDMS) which has been doped, impregnated, and/or coated with electroconductive material.

In an example, electromagnetic energy can be transmitted to light emitters through undulating wires, conductive threads, or conductive yarns. In an example, light emitters can receive electrical power through undulating (e.g. undulating, wavy, zigzag, and/or sinusoidal) wires (e.g. microwires or nanowires). In an example, light emitters can receive electrical power through channels comprising an elastomeric polymer which has been embedded, impregnated, and/or coated with electroconductive material. In an example, light emitters can receive power from undulating (e.g. sinusoidal) electroconductive pathways.

In an example, a cup can comprise a plurality of endpoints (or side locations) on a plurality of optical fibers which emit light, wherein these optical fibers transmit this light into the optical layer from light sources (e.g. LEDs) outside the optical layer. In an example, a cup can further comprise elastic optical fibers which guide light to light-exiting points. In an example, a cup can further comprise optical fibers which emit light from their ends at a plurality of light-exiting points around the interior concavity of a cup. In an example, light from light emitters (e.g. LEDs) can be distributed across a concave inner surface via a plurality of optical fibers (e.g. light-conducting fibers, tubes, channels, or threads).

In an example, a bra for optical detection of abnormal breast tissue can comprise an air-gap-reducing layer. In an example, a cup can comprise three layers: an inner air-gap-reducing layer (closest to the surface of the breast), an optical layer, and an outer structural layer (farthest from the surface of the breast). In an example, a first part of an air-gap-reducing layer on a first side of a virtual plane can be pushed closer to a second part of an air-gap-reducing layer on a second (e.g. opposite) side of the virtual plane when the expandable layer is expanded and a cup is changed from its first (unexpanded) configuration to its second (expanded) configuration. In an example, an air-gap-reducing layer can be sufficiently soft, compressible, elastomeric, and/or flexible to conform to the shape of the breast under gentle pressure from the cup of a bra. In an example, an air-gap-reducing layer of a cup can be made with a transparent elastomeric material.

In an example, an air-gap-reducing layer of a cup can be made from an aerogel. In an example, an air-gap-reducing layer of a cup can be made from a silicon composite. In an example, an air-gap-reducing layer of a cup can be made from a gelatin. In an example, an air-gap-reducing layer of a cup can be made from chitosan. In an example, an air-gap-reducing layer of a cup can be made from poly-vinyl Pyrrolidone (PVP). In an example, an air-gap-reducing layer of a cup can be made from polyvinyl Alcohol (PVA). In an example, an air-gap-reducing layer of a cup can be made from a homopolymeric polymer gel. In an example, an air-gap-reducing layer of a cup can be made from poly-hydroxy-ethyl-methyl Acrylate (PHEMA). In an example, an air-gap-reducing layer of a cup can be made from polyamino acid. In an example, an air-gap-reducing layer of a cup can be the most elastic layer of the cup.

In an example, an air-gap-reducing layer of a cup can comprise an acrylic elastomer. In an example, an air-gap-reducing layer of a cup can have one or more optical parameters (e.g. optical absorption coefficient, optical scattering coefficient, and/or anisotropy factor) a value which is within plus or minus 20% of the mean value for normal breast tissue. In an example, an air-gap-reducing layer of a cup can have an optical scattering coefficient with a value within one standard deviation of the mean value (range) for normal breast tissue. In an example, an air-gap-reducing layer of a cup can have an optical absorption coefficient with a value within one standard deviation of the mean value (range) for normal breast tissue.

In an example, an air-gap-reducing layer of a cup can have a Young's modulus between 0.50 and 4.00. In an example, an air-gap-reducing layer of a cup can one or more optical parameters (e.g. optical absorption coefficient, optical scattering coefficient, and/or anisotropy factor) which are each within one standard deviation of the mean parameter value for normal breast tissue. In an example, part of an air-gap-reducing layer can have a first degree of concavity when a cup is in the first (unexpanded) configuration and a second degree of concavity with the cup is in the second (expanded) configuration, wherein the second degree is less than the first degree. In an example, the inner air-gap-reducing layer can be sufficiently conformable, flexible, and thick that it can greatly reduce (or even eliminate) air gaps between the optical layer and the surface of the breast, even for breasts with different sizes and shapes. In an example, the thickness, elasticity, and/or compressibility of an air-gap-reducing layer can be adjusted by inflation. In an example, the thickness, elasticity, and/or compressibility of an air-gap-reducing layer can be adjusted by pumping fluid (or gel) into the layer or out of the layer.

In an example, a device can further comprise an inclinometer which enables positioning the device in the same orientation relative to a vertical plane in repeated uses (e.g. wearing the device at different times). In an example, this device can further comprise an inclinometer in order to register (e.g. align) the device relative to a vertical plane. In an example, if this device is embodied in a bra, then one or more additional components can be located on the back strap of the bra. In an example, this device can further comprise one or more other components selected from the group consisting of: power source, data processor, wireless data transmitter, wireless data receiver, air pump, and liquid pump. In a smart bra embodiment, one or more of these other components can be located on the back (e.g. strap) portion of the bra.

In an example, a data processor can receive data from light receivers on a bra. In an example, a smart bra can further comprise a local data processor which is located on the posterior portion of a smart bra. In an example, a smart bra can further comprise a data processor. In an example, a smart bra can further comprise a local data processor which is in electronic communication with a remote data processor. In an example, analysis of changes in light intensity and/or spectral distribution caused by light transmission through and/or reflection from breast tissue can be analyzed in a remote data processor. In an example, data from light receivers can be transmitted to a separate and/or remote data processor for spectroscopic analysis to identify changes in breast tissue composition and/or to identify abnormal breast tissue. In an example, a bra can include an energy source such as a battery. In an example, a power source (e.g. battery) can be located on the back strap of a smart bra. In an example, a smart bra can further comprise a power source.

In an example, a cup component can be a modular and/or removable component of a smart bra which can be removed before the bra is washed. In an example, a device with optical sensors to detect abnormal breast tissue can be an insert which is placed between the cup of a bra and a breast. In an example, a device with optical sensors to detect abnormal breast tissue can be inserted into a bra cup. In an example, a device with optical sensors to detect abnormal breast tissue can be embodied in a patch or bandage which is gently adhered to a breast. In an example, a device with optical sensors to detect abnormal breast tissue can be removably inserted into the cup on a bra.

In an example, a device with optical sensors to detect abnormal breast tissue can be removably attached to the concave interior of a cup of a bra. In an example, a device with optical sensors to detect abnormal breast tissue can be worn between a bra cup and a breast. In an example, a modular device (e.g. bra insert) can be rotated between use on the right-side and then the left-side of a conventional bra. In an example, a system can comprise a specialized bra with attachment mechanisms (e.g. hook and loop fabric, snap, or clip) on the interior of cups to which an insert with optical sensors is removably attached.

In an example, a bra and/or cup can further comprise marks, indicators, openings, or sensors which are configured to help register and/or align it relative to anatomy of the breast. In an example, a cup or cup insert can be rotated and/or shifted to align it with anatomical locations and/or features of a breast in order to register and/or align optical scans taken at different times (e.g. when the person wears it at different times). In an example, a cup or cup insert can be rotated for use in different orientations on the same breast. In an example, a cup or cup insert can be rotated and/or flipped from right breast use to left breast use. In an example, an optical layer of a cup can be rotated relative to the structural layer of a cup in order to adjust the portions of the breast which are compressed and/or optically scanned. In an example, one or more layers of a cup or cup insert can be rotated.

In an example, changes in light emitted from light emitters and received by the light receivers which are caused by transmission of the light through the breast can be analyzed to detect abnormal breast tissue and/or identify attributes of the abnormal breast tissue. In an example, changes in the intensity (e.g. amplitude) of light emitted from light emitters and received by light receivers which are caused by the transmission of the light through breast tissue can be analyzed to image breast tissue. In an example, changes in the intensity (e.g. amplitude) of light emitted from light emitters and received by light receivers which are caused by transmission of the light through breast tissue can be analyzed to evaluate the size, shape, density, and/or location of abnormal breast tissue. In an example, light emitted by the light emitters can be received by the light receivers after this light has been transmitted through breast tissue.

In an example, a smart bra can detect and/or image abnormal tissue by analyzing light reflection. In an example, changes in the direction of light caused by its reflection from breast tissue can be analyzed to create an image of the breast tissue. In an example, light emitted by the light emitters can be received by the light receivers after this light has been reflected within breast tissue. In an example, light from light emitters which has been transmitted through and/or reflected from breast tissue and received by the light receivers is analyzed to detect and/or image abnormal breast tissue.

In an example, a smart bra can detect abnormal tissue via Time Domain (TD) optical analysis. In an example, a smart bra can detect abnormal tissue via Continuous Wave (CW) optical analysis. In an example, a smart bra can use Raman scattering to analyze the molecular composition of breast tissue, detect abnormal breast tissue, evaluate the size and shape of abnormal breast tissue, identify selected biometric parameters in breast tissue, identify the location of abnormal breast tissue, and/or image breast tissue.

In an example, changes in the intensity and/or spectrum of light caused by its transmission through breast tissue from light emitters in the first part of the optical layer to light receivers in the second part of the optical layer through breast tissue can be analyzed to detect and/or image abnormal tissue. In an example, changes in the intensity and/or spectral distribution of light caused by transmission through breast tissue can be analyzed to create a (3D) image which shows variation in breast tissue structure. In an example, changes in the spectrum (e.g. spectral distribution) of light emitted from light emitters and received by light receivers which are caused by transmission of the light through breast tissue can be analyzed to evaluate the size, shape, density, and/or location of abnormal breast tissue. In an example, changes in the spectrum (e.g. spectral distribution) of light emitted from light emitters and received by light receivers which are caused by transmission of the light through breast tissue can be analyzed to detect abnormal breast tissue. In an example, changes in the spectrum of light caused by its reflection from breast tissue can be analyzed to identify the levels, concentrations, and/or locations of specific biological substances (e.g. markers associated with abnormal tissue) in the breast tissue.

In an example, a smart bra can use Near-InfraRed Spectroscopy (NIRS) to analyze the molecular composition of breast tissue, detect abnormal breast tissue, evaluate the size and shape of abnormal breast tissue, identify selected biometric parameters in breast tissue, identify the location of abnormal breast tissue, and/or image breast tissue. In an example, spectroscopic analysis of near-infrared light which has been reflected from breast tissue can be done to identify the sizes, configurations, and/or locations of specific biological structures associated with abnormal tissue. In an example, spectroscopic analysis of light which has been transmitted through and/or reflected from breast tissue can be done to identify the levels, concentrations, and/or locations of specific biological substances (e.g. markers associated with abnormal tissue) in the breast tissue. In an example, spectroscopic analysis of light which has been reflected from breast tissue can be done to identify the levels, concentrations, and/or locations of specific biological substances (e.g. markers associated with abnormal tissue) in the breast tissue.

In an example, light which has been transmitted through and/or reflected from breast tissue can be analyzed to detect and/or image the locations, sizes, and/or configurations of vasculature sprouting in breast tissue. In an example, light which has been transmitted through and/or reflected from breast tissue can be analyzed to detect and/or image levels, concentrations, and/or locations of oxygenated hemoglobin in breast tissue. In an example, light which has been transmitted through and/or reflected from breast tissue can be analyzed to detect and/or image levels, concentrations, and/or locations of hemoglobin in breast tissue. In an example, light which has been transmitted through breast tissue between different pairs of light emitters and light receivers (at different times) can be triangulated in order to identify the presence, composition, shape, size, and/or location of abnormal tissue. In an example, a smart bra can be worn periodically in order to obtain a longitudinal time series of optical scans of breast tissue.

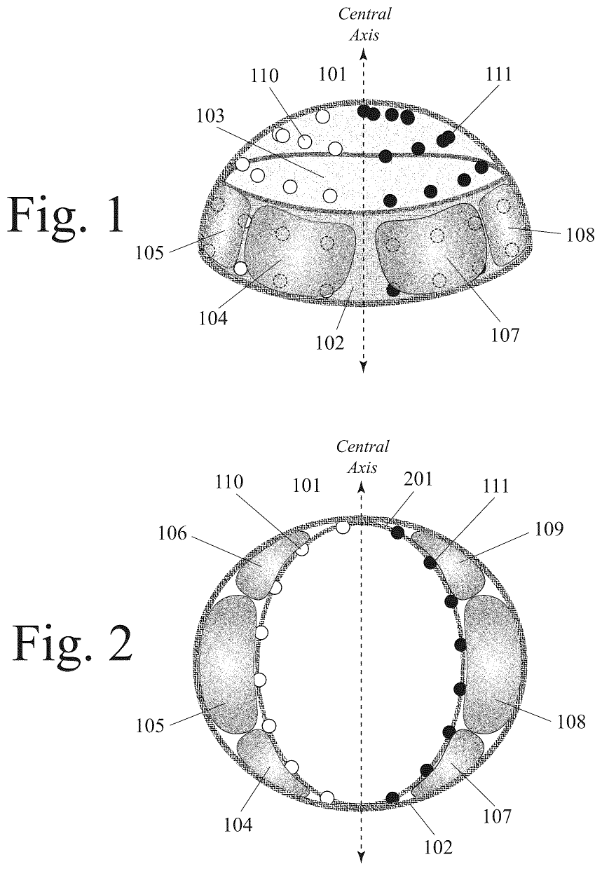

FIGS. 1 through 6 show six views, from two different perspectives and at three different times, of a smart bra cup which enables optical detection of abnormal breast tissue (e.g. breast cancer). FIG. 1 shows a semi-transparent lateral (e.g. left side or right side) view of the cup at a first time, before the cup has been expanded to gently compress a breast for optical scanning. FIG. 2 shows, also at this first time, a semi-transparent frontal (e.g. distal-to-proximal) view of a lateral cross-section of the cup. In this disclosure, distal means farther from the chest wall and proximal means closer to the chest wall, when the cup is worn on a person's breast.

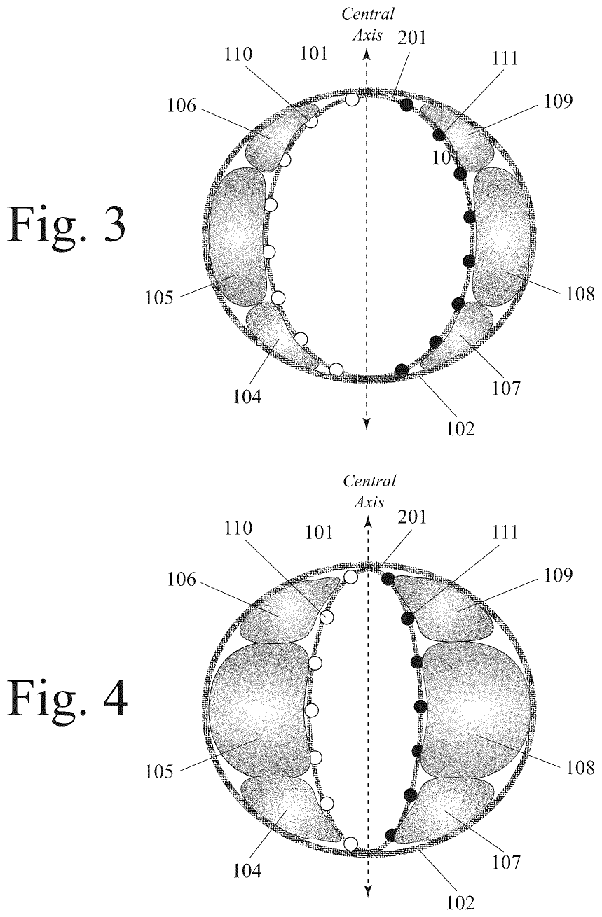

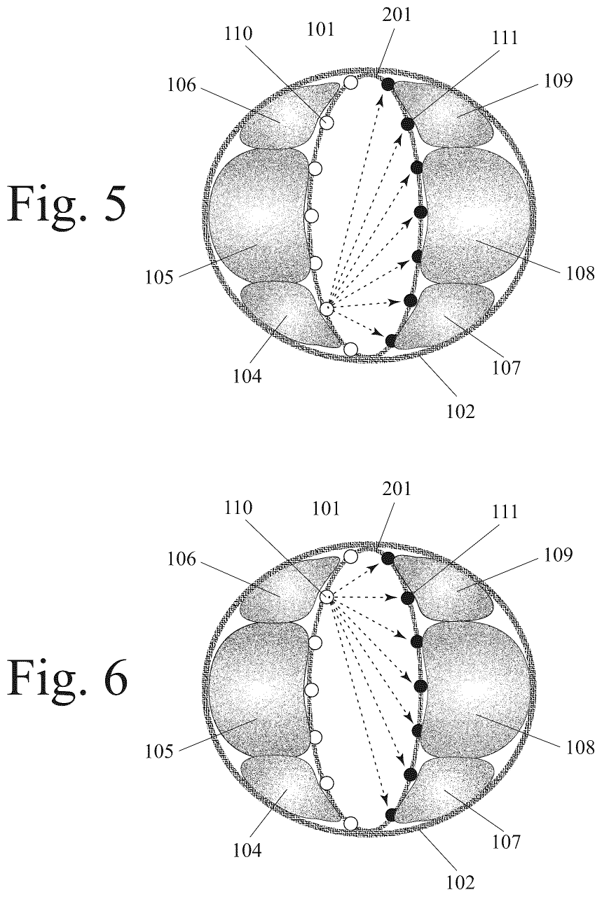

FIG. 3 shows the semi-transparent lateral view of the cup at a second time, after the cup has been expanded to gently compress a breast for optical scanning, but before optical scanning occurs. FIG. 4 shows, also at the second time, the semi-transparent frontal view of the lateral cross-section of the cup. FIGS. 5 and 6 show the semi-transparent frontal view of the lateral cross-section of the cup at a third time and a fourth time, while the breast is being optically scanned.

The bra cup which is shown in FIGS. 1 through 6 comprises: an overall cup 101 which is configured to be worn on a person's breast; a proximal portion 102 of the cup; a distal portion 103 of the cup, wherein the distal portion is farther from the chest wall than the proximal portion (when the cup is worn on a person's breast), and wherein the distal portion is more flexible or elastic than the proximal portion; a first set of one or more expandable chambers (104, 105, and 106) on a first side of a virtual plane which passes through the cup, wherein the virtual plane is along a vertical central axis of the cup in this example; a second set of one or more expandable chambers (107, 108, and 109) on a second side of the virtual plane; an array of light emitters (including light emitter 110), wherein at least some of the light emitters are configured to be between the first set of one or more expandable chambers and the breast; an array of light receivers (including light receiver 111), wherein at least some of the light receivers are configured to be between the second set of one or more expandable chambers and the breast; and an inner component (e.g. an inner ring or layer) 201 between the light emitters or light receivers and the expandable chambers; wherein changes in light emitted from the light emitters and received by the light receivers which are caused by transmission of the light through the breast are analyzed to detect abnormal breast tissue and/or identify attributes of the abnormal breast tissue.

In an example, a bra for optical detection of abnormal breast tissue can comprise: a cup which is configured to be worn on a person's breast; wherein the cup includes a proximal portion; wherein the cup includes a distal portion, wherein the proximal portion is configured to be closer to the person's chest wall than the distal portion the cup is worn on the person's breast, and wherein the distal portion is more flexible or elastic than the proximal portion; a first set of one or more expandable chambers on a first side of a virtual plane which passes through the cup; a second set of one or more expandable chambers on a second side of the virtual plane; an array of light emitters, wherein at least some of the light emitters are configured to be between the first set of one or more expandable chambers and the breast; and an array of light receivers, wherein at least some of the light receivers are configured to be between the second set of one or more expandable chambers and the breast; wherein changes in light emitted from the light emitters and received by the light receivers which are caused by interaction between the light and the breast are analyzed to detect abnormal breast tissue.

In an example, expandable chambers can be expanded by being filled with a flowable substance. In an example, the flowable substance can be a gas. In an example, the flowable substance can be a liquid. In an example, the virtual plane can be vertical when the person is standing. In an example, the virtual plane can be oblique, spanning from an upper outer quadrant of the breast to a lower inner quadrant of a breast. In an example, the proximal portion can have a ring, band, column-section, or frustal shape. In an example, a proximal perimeter of the proximal portion can have a circular, elliptical, oval, teardrop, or crescent shape.

In an example, an expandable chamber between two other expandable chambers on a side of a virtual plane can be larger and/or expanded more than the two other expandable chambers. In an example, a cup can further comprise an inner component located between light emitters and expandable chambers. In an example, a cup can further comprise an inner component located between light receivers and expandable chambers. In an example, each expandable chamber can be connected to a separate tube. In an example, an expandable chamber can have undulating, variable-width sections along its length. In an example, light from light emitters can be transmitted through the breast to the light receivers. In an example, light from light emitters can be reflected from the breast to the light receivers.

FIGS. 1 and 2 show this cup at a first point in time, before expandable chambers have been expanded. At this first time, the inner component and the arrays of light emitters and receivers surround an elliptical (or oval) shaped inner space which is configured to receive a breast in an uncompressed state. In this figure, light emitters including 110 are shown as open circles. In this example, light emitters are distributed along a side of an arcuate inner component (e.g. inner ring or layer) located to the left of a virtual plane. In this figure, light receivers including 111 are shown as filled circles. In this example, light receivers are distributed along the side of the arcuate inner component (e.g. inner ring or layer) located to the right of the virtual plane.

In this example, light emitters and light receivers are on opposite sides of a breast. Light from light emitters is received by light receivers after it has been transmitted through the breast tissue. Changes in light caused by interaction with breast tissue are used to detect abnormal breast tissue. Changes in light can also be used to identify attributes of abnormal breast tissue such as location, size, shape, and composition. This can be an advantage over traditional mammography which does not analyze the molecular composition of breast tissue.

FIGS. 3 and 4 show this cup at a second point in time, after the expandable chambers have been expanded. At this second time, the elliptical (or oval) shaped inner space has been narrowed by expansion of the chambers, gently compressing the breast within it. Gentle compression of the breast improves the accuracy of optical scanning to detect abnormal tissue in the breast. In an example, expandable chambers can be expanded by a manual pump which is operated by the person wearing the bra so that that person can easily avoid pain from over-compression by stopping expansion. In another example, a bra can include pressure sensors which control a pump, wherein expansion is stopped when pressure levels reach a selected level. In an example, an expandable chamber which is central to one side of a cup can be larger, or expanded more, than other expandable chambers on that side of the cup.

FIGS. 5 and 6 show this cup at two subsequent times, while the breast is being optically scanned. In this figure, light rays which are transmitted through breast tissue from a light emitter to light emitters are represented by dotted-line arrows. FIGS. 5 and 6 show two light ray pathways at two different times, but these are only a subset of the many different light pathways which occur between various combinations of light emitters and receivers during an optical scan. In an example, different light emitters can be activated (e.g. pulsed) at different times (e.g. sequentially). Collecting data on changes in transmitted light from a large number of different light pathways (e.g. light emitter and light receiver combinations) enables more accurate detection of abnormal tissue and identification of tissue attributes (such as location, size, shape, and composition). In an example, data patterns concerning light changes in multiple pathways can be analyzed by machine learning and/or artificial intelligence to detect abnormal tissue and identify tissue attributes.

In an example, there can be right-side and left-side versions of this bra cup. In an example, a left-side version of a cup can be symmetric to a right-side version of the bra cup. In an example, a cup can be permanently integrated into a smart bra. In an example, a cup component can be a modular and/or removable component of a smart bra which can be removed before the bra is washed. In an example, this device (e.g. cup) can be a modular device (e.g. bra insert) which is inserted between the cup of conventional bra and a breast. In an example, a modular device (e.g. bra insert) can be used on the right-side and then on the left-side of a conventional bra. In an example, a modular device (e.g. bra insert) can be rotated between use on the right-side and then the left-side of a conventional bra.

In an example, expandable chambers can be expanded by being filled with a flowable substance, such as a gas (e.g. air) or liquid (e.g. water). In an example, a cup can further comprise tubes which convey a flowable substance (e.g. air or liquid) from a pump into the expandable chambers. In an example, the pump can be permanently integrated into (the back strap of) the smart bra. In an example, the pump can be separate and only connected to the smart bra when the expandable chambers need to be expanded. In an example, the pump can be manually operated. In an example, the pump can be automatic, but expansion can be limited based on data from sensors (e.g. pressure or motion sensors). In another example, expandable chambers can be expanded by electromagnetic actuators.

In an example, a first set of expandable chambers and also light emitters can be located on one side of a virtual plane and a second set of expandable chambers and also light receivers can be located on the other side of the virtual plane. In an example, this virtual plane can be vertical (e.g. spanning between outer quadrants of a breast and lower quadrants of the breast) when a person wearing the bra is standing. In another example, a virtual plane can be oblique (e.g. spanning from an upper outer quadrant of a breast to a lower inner quadrant of a breast). A design with an oblique virtual plane can be better for detecting abnormal tissue in the upper outer quadrant of a breast and the Tail of Spence. These areas are more prone to develop malignancy than other areas of the breast or chest wall.

In an example, the proximal portion of a cup which is closer to the chest wall can have a ring, band, column-section, or frustal shape. In an example, the proximal perimeter of this proximal portion can have a circular, elliptical, oval, teardrop, or crescent shape. In an example, a proximal portion of a cup can be less flexible and more rigid than a distal portion of a cup so that it resists outward expansion when expandable chambers are expanded. In an example, a proximal portion of a cup can be reinforced with wires, strips, or bands to make it less flexible and more rigid. In an example, a proximal portion of a cup can span between one quarter and two-thirds of the proximal-to-distal distance of the overall cup.

In an example, a distal portion of a cup which is farther from the chest wall can have a hemispherical, semi-ellipsoidal, paraboloidal, dome, or other concave shape. In an example, a distal portion of a cup can be more flexible and/or elastic than the proximal portion of the cup. This it allows forward and/or outward expansion of a breast when expandable chambers are expanded and a breast is gently compressed. In another embodiment, a cup can comprise only a proximal portion, with its distal side being completely open to allow maximum forward expansion of a breast during compression.

In an example, there can be a plurality of expandable chambers on each side of a virtual plane which passes through a cup. In this example, there are three expandable chambers on each side of the virtual plane. In an example, an expandable chamber between two other expandable chambers on a side of a virtual plane can be larger (and/or expanded more) than the two other expandable chambers on the same side. This can help to gently compress the wider central portion of a breast more than peripheral portions of the breast. This helps to flatten the breast for more accurate optical scanning. It is anticipated that this breast compression will be less uncomfortable than compression in traditional mammography because: uniform breast flatness is not as critical in optical scanning as in traditional mammography; and the bra cup does not compress a breast with rigid rectangular surfaces as in traditional mammography.

In an example, light emitters can be on one side of a virtual plane which passes through a cup and light receivers can be on the other side of the virtual plane. In this configuration, light emitted by the light emitters is received by the light receivers after this light has been transmitted through breast tissue. In a second example, both light emitters and light receives can be on the same side of a virtual plan. In this second example, light emitted by the light emitters is received by the light receivers after this light has been reflected within breast tissue. In an example, optical components, which each comprise one or more light emitters and one or more light receivers, can be distributed on both sides of a virtual plane.

In an example, a cup can also comprise an inner component (e.g. an inner ring or layer) located between light emitters or receivers and expandable chambers. This inner component (e.g. inner ring or layer) can help to keep light emitters and receivers in the proper locations and orientations relative to a breast as the expandable chambers are expanded. In an example, a cup can also comprise electroconductive pathways which are connected to light emitters and light receivers. In an example, these electroconductive pathways can be undulating and/or elastic so that they do not constrain expansion of expandable chambers. Relevant variations discussed elsewhere in this disclosure or in priority-linked disclosures can also be applied to this example.

FIG. 7 shows a semi-transparent frontal view of the lateral cross-section of the same cup that was introduced in FIGS. 1 through 6 but is now shown in context as part of a smart bra 701. As was the case in FIGS. 1 through 6, the virtual plane which separates two sets of expandable chambers (and also the light emitters vs, light receivers) is vertical.

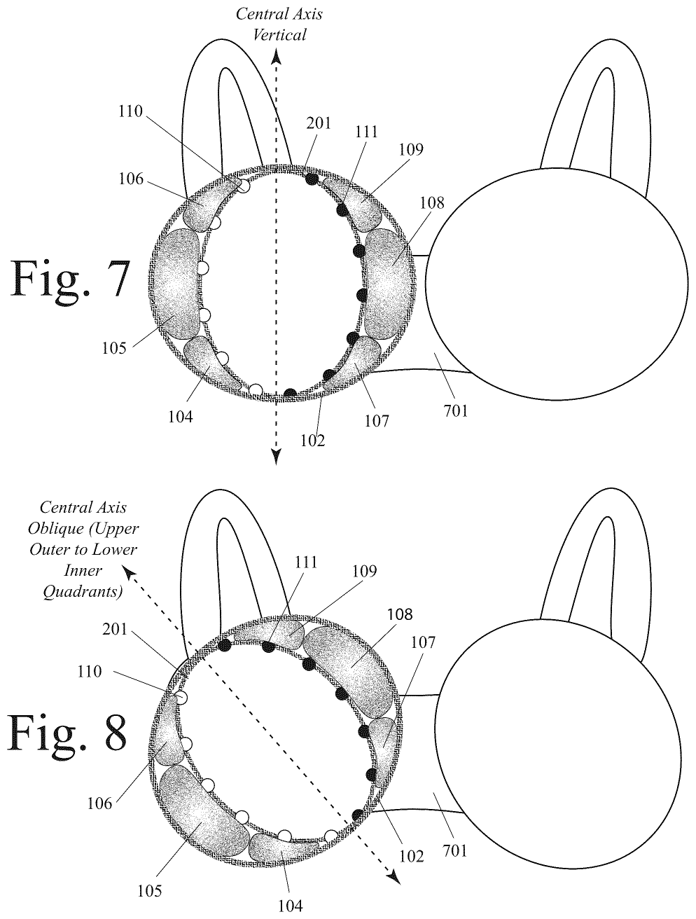

FIG. 8 shows this same cup, but now the virtual plane is oblique—spanning from the upper outer quadrant of the breast to the lower inner quadrant of the breast. This oblique configuration for the virtual plane can enable the cup to more accurately detecting abnormal tissue in the upper outer quadrant of the breast and also the associated Tail of Spence. These areas are disproportionately prone to malignancy as compared to other areas of the breast.

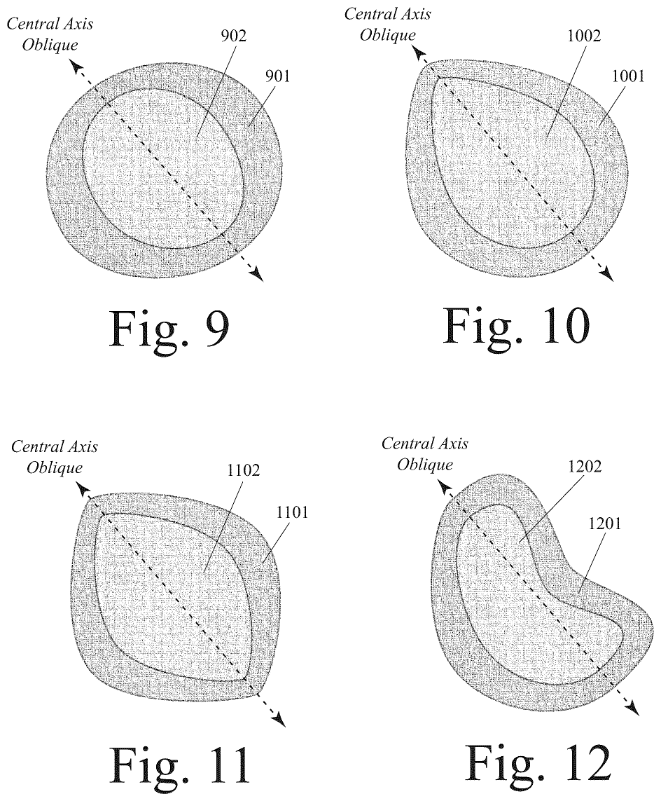

FIGS. 9 through 12 shows frontal (e.g. distal-to-proximal) views of bra cups with different shapes. FIG. 9 shows a front view of a bra cup with a proximal portion 901 and a distal portion 902. In this example, the bra cup has an elliptical, oval, and/or oblate-circular shape. In an example, a bra cup can have an elliptical, oval, circular, oblate-circular, and/or globular shape. In an example, the perimeter of a cross-section of a proximal portion of a bra cup can have an elliptical, oval, circular, and/or oblate-circular shape. In an example, the perimeter of a cross-section of a distal portion of a bra cup can have an elliptical, oval, circular, and/or oblate-circular shape. In this example, the cup has an oblique virtual plane which spans from the upper outer quadrant of a breast to the lower inner quadrant of the breast.

FIG. 10 shows a front view of a bra cup with a proximal portion 1001 and a distal portion 1002. In this example, the bra cup has a teardrop shape. In an example, a bra cup can have an teardrop shape. In an example, the perimeter of a cross-section of a proximal portion of a bra cup can have a teardrop shape. In an example, the perimeter of a cross-section of a distal portion of a bra cup can have a teardrop shape. In this example, the cup has an oblique virtual plane which spans from the upper outer quadrant of a breast to the lower inner quadrant of the breast.

FIG. 11 shows a front view of a bra cup with a proximal portion 1101 and a distal portion 1102. In this example, the bra cup has a football shape. In an example, the perimeter of a cross-section of a proximal portion of a bra cup can have a football shape. In an example, the perimeter of a cross-section of a distal portion of a bra cup can have a football shape. In this example, the cup has an oblique virtual plane which spans from the upper outer quadrant of a breast to the lower inner quadrant of the breast.

FIG. 12 shows a front view of a bra cup with a proximal portion 1201 and a distal portion 1202. In this example, the bra cup has a kidney-bean and/or banana shape. In an example, the perimeter of a cross-section of a proximal portion of a bra cup can have a kidney-bean and/or banana shape. In an example, the perimeter of a cross-section of a distal portion of a bra cup can have a kidney-bean and/or banana shape. In this example, the cup has an oblique virtual plane which spans from the upper outer quadrant of a breast to the lower inner quadrant of the breast.

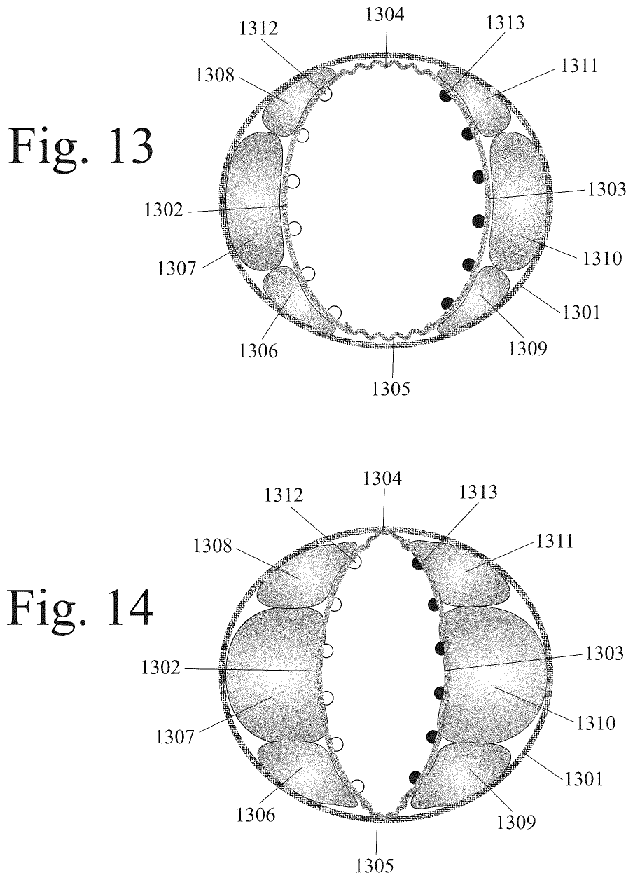

FIGS. 13 and 14 show two semi-transparent frontal (e.g. distal-to-proximal) views, at two different times, of a lateral cross-section of a cup with a partially-undulating inner component (e.g. inner ring or band) between light emitters or receivers and expandable chambers. FIG. 13 shows the cup at first time, before the expandable chambers have been expanded. FIG. 14 shows the cup at second time, after the expandable chambers have been expanded. This cup differs from the cup shown in FIGS. 1 through 6 because portions of the inner component (e.g. inner ring or band) which are closer to the virtual plane are undulating and/or pleated. These undulating and/or pleated portions are more flexible and less rigid than the other portions of the inner component. This enables the inner component (e.g. inner ring or band) to compress the breast more effectively, without distorting the locations and orientations of light emitters and receivers relative to the breast.

The bra cup which is shown in FIGS. 13 and 14 comprises: a proximal portion 1301 of the cup; a distal portion of the cup (not shown in this cross-sectional view), wherein the distal portion is farther from the chest wall than the proximal portion (when the cup is worn on a person's breast), and wherein the distal portion is more flexible or elastic than the proximal portion; a first set of one or more expandable chambers (1306, 1307, and 1308) on a first side of a virtual plane which passes through the cup; a second set of one or more expandable chambers (1309, 1310, and 1311) on a second side of the virtual plane; an array of light emitters (including light emitter 1312), wherein at least some of the light emitters are configured to be between the first set of one or more expandable chambers and the breast; an array of light receivers (including light receiver 1313), wherein at least some of the light receivers are configured to be between the second set of one or more expandable chambers and the breast; and an inner component (e.g. an inner ring or layer) between the light emitters or light receivers and the expandable chambers, wherein the inner component further comprises a first portion 1302 on a first side of the virtual plane, a second portion 1303 on a second side of the virtual plane, and two undulating and/or pleated portions (1304 and 1305) which cross the virtual plane; wherein changes in light emitted from the light emitters and received by the light receivers which are caused by transmission of the light through the breast are analyzed to detect abnormal breast tissue and/or identify attributes of the abnormal breast tissue.

In an example, the inner component (including portions 1302, 1303, 1304, and 1305) can have a generally elliptical, oval, circular, or oblate-circular shape before expansion of the expandable chambers and a football shape after expansion of the expandable chambers. Relevant variations discussed elsewhere in this disclosure or in priority-linked disclosures can also be applied to this example.

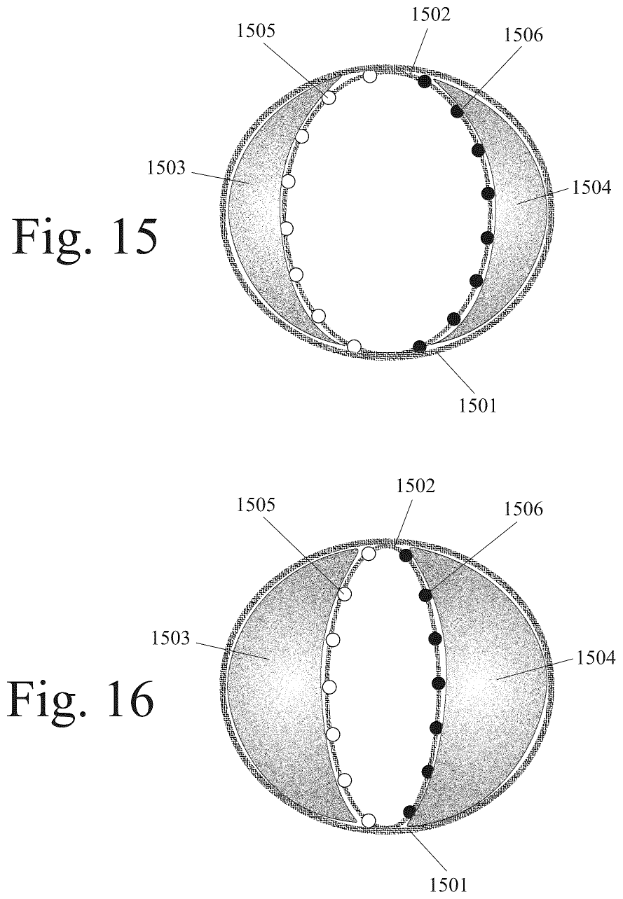

FIGS. 15 and 16 show two semi-transparent frontal (e.g. distal-to-proximal) views, at two different times, of a lateral cross-section of a cup, wherein there is only one expandable chamber on each side of a virtual plane. FIG. 15 shows the cup at first time, before the expandable chambers have been expanded. FIG. 16 shows the cup at second time, after the expandable chambers have been expanded. In this example, both expandable chambers have a crescent, partial moon, and/or banana shape. In this example, expandable chambers on one side of a virtual plane are symmetric with respect to expandable chambers on the other side of the virtual plane.

The bra cup which is shown in FIGS. 15 and 16 comprises: a proximal portion 1501 of the cup; a distal portion of the cup (not shown in this cross-sectional view), wherein the distal portion is farther from the chest wall than the proximal portion (when the cup is worn on a person's breast), and wherein the distal portion is more flexible or elastic than the proximal portion; a first expandable chamber 1503 on a first side of a virtual plane which passes through the cup; a second expandable chamber 1504 on a second side of the virtual plane; an array of light emitters (including light emitter 1505), wherein at least some of the light emitters are configured to be between the first expandable chamber and the breast; an array of light receivers (including light receiver 1506), wherein at least some of the light receivers are configured to be between the second expandable chamber and the breast; and an inner component (e.g. an inner ring or layer) 1502 between the light emitters or light receivers and the expandable chambers; wherein changes in light emitted from the light emitters and received by the light receivers which are caused by transmission of the light through the breast are analyzed to detect abnormal breast tissue and/or identify attributes of the abnormal breast tissue. Relevant variations discussed elsewhere in this disclosure or in priority-linked disclosures can also be applied to this example.

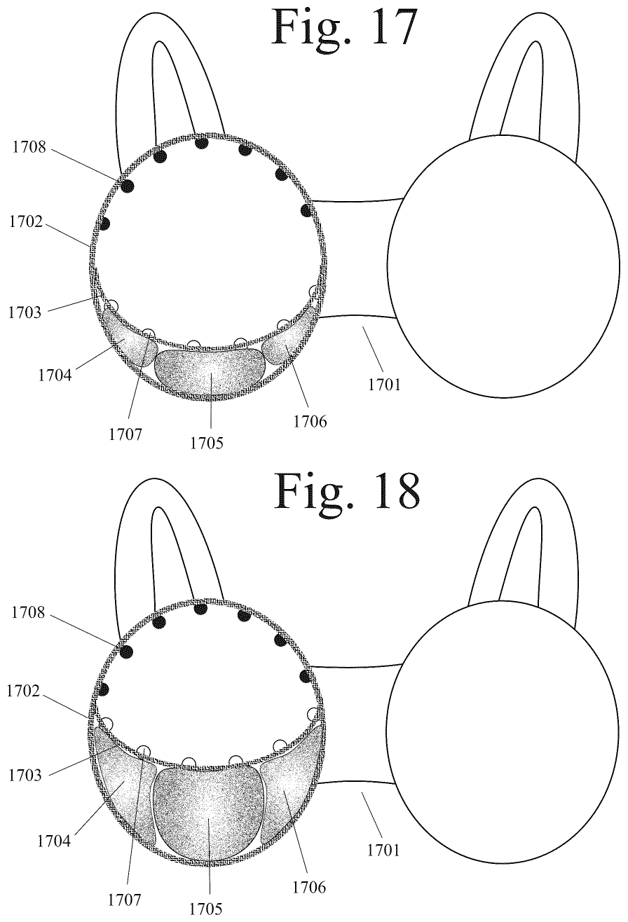

FIGS. 17 and 18 show two semi-transparent frontal (e.g. distal-to-proximal) views, at two different times, of a lateral cross-section of a cup (on a bra) wherein there are expandable chambers on only one side of a virtual plane which passes through the cup. FIG. 17 shows the cup at first time, before the expandable chambers have been expanded. FIG. 18 shows the cup at second time, after the expandable chambers have been expanded. In this example, there are expandable chambers only on the side of the cup where light emitters are located. In another example, there can be expandable chambers only on the side of the cup where light detectors are located.

The bra cup which is shown in FIGS. 17 and 18 comprises: a proximal portion 1702 of the cup on a bra 1701; a distal portion of the cup (not shown in this cross-sectional view), wherein the distal portion is farther from the chest wall than the proximal portion (when the cup is worn on a person's breast), and wherein the distal portion is more flexible or elastic than the proximal portion; expandable chambers (1704, 1705, and 1706) a first side of a virtual plane which passes through the cup; an array of light emitters (including light emitter 1707), wherein at least some of the light emitters are configured to be between the expandable chambers and the breast; an array of light receivers (including light receiver 1708) configured to be between the proximal portion and the breast; and an inner component (e.g. an inner ring or layer) 1703 between the light emitters and the expandable chambers; wherein changes in light emitted from the light emitters and received by the light receivers which are caused by transmission of the light through the breast are analyzed to detect abnormal breast tissue and/or identify attributes of the abnormal breast tissue. Relevant variations discussed elsewhere in this disclosure or in priority-linked disclosures can also be applied to this example.

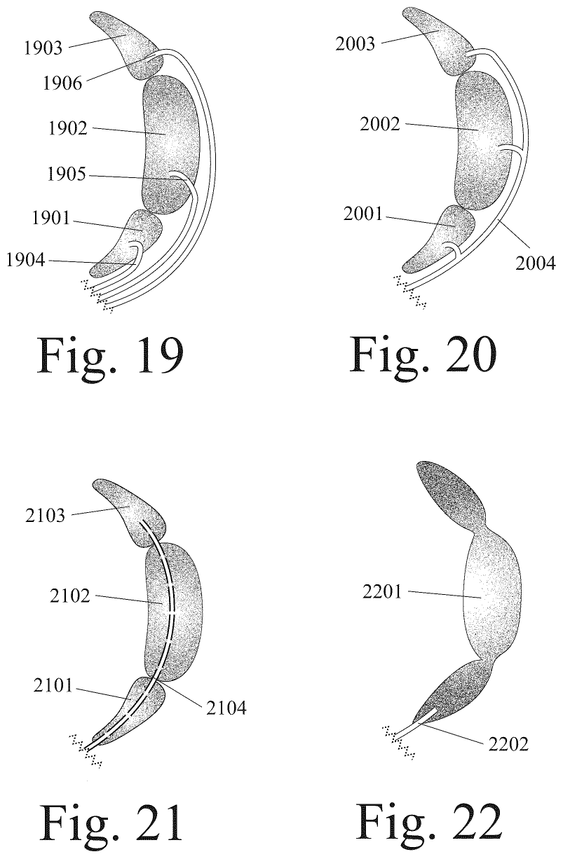

FIGS. 19 through 22 show examples of four different configurations of expandable chambers and tubes which are in fluid communication with those chambers. In an example, expandable chambers can be expanded by being filled with a flowable substance which is inserted into them through these tubes. In an example, this flowable substance can be a gas (e.g. air) or a liquid (e.g. water).

FIG. 19 shows a configuration of expandable chambers and tubes wherein each expandable chamber is connected to a separate tube. This enables independent (e.g. differential and/or sequential) expansion of different (e.g. selected) expandable chambers. The configuration of expandable chambers and tubes in FIG. 19 comprises: a plurality of expandable chambers (1901, 1902, and 1903); and a plurality of tubes (1904, 1905, and 1906), wherein the expandable chambers are expanded by being filled with a flowable substance (e.g. a gas or liquid) delivered through the tubes, and wherein each expandable chamber is connected to a separate tube, thereby enabling independent (e.g. differential and/or sequential) expansion of different expandable chambers.

FIG. 20 shows a configuration of expandable chambers and a tube, wherein all expandable chambers (on the same side of a virtual plane) are connected to a common tube. The configuration of expandable chambers and a tube in FIG. 20 comprises: a plurality of expandable chambers (2001, 2002, and 2003); and a tube 2004, wherein the expandable chambers are expanded by being filled with a flowable substance (e.g. a gas or liquid) delivered through the tube.

FIG. 21 shows a configuration of expandable chambers and a tube wherein all expandable chambers (on the same side of a virtual plane) are connected to a common tube and wherein the tube goes through the interiors of the expandable chambers. In this example, the tube has a number of holes along its length which enable fluid communication between the interiors of the expandable chambers. The configuration of expandable chambers and a tube in FIG. 21 comprises: a plurality of expandable chambers (2101, 2102, and 2103); and a tube 2104, wherein the expandable chambers are expanded by being filled with a flowable substance (e.g. a gas or liquid) delivered through the tube, and wherein the tube goes through the interiors of the expandable chambers.

FIG. 22 shows an expandable chamber and a tube, wherein the expandable chamber further comprises undulating (e.g. width-varying and/or sinusoidal) sections along its length. The configuration of an expandable chamber and a tube in FIG. 22 comprises: an expandable chamber 2201 with undulating-width sections along its length; and a tube 2202, wherein the expandable chamber is expanded by being filled with a flowable substance (e.g. a gas or liquid) delivered through the tube.

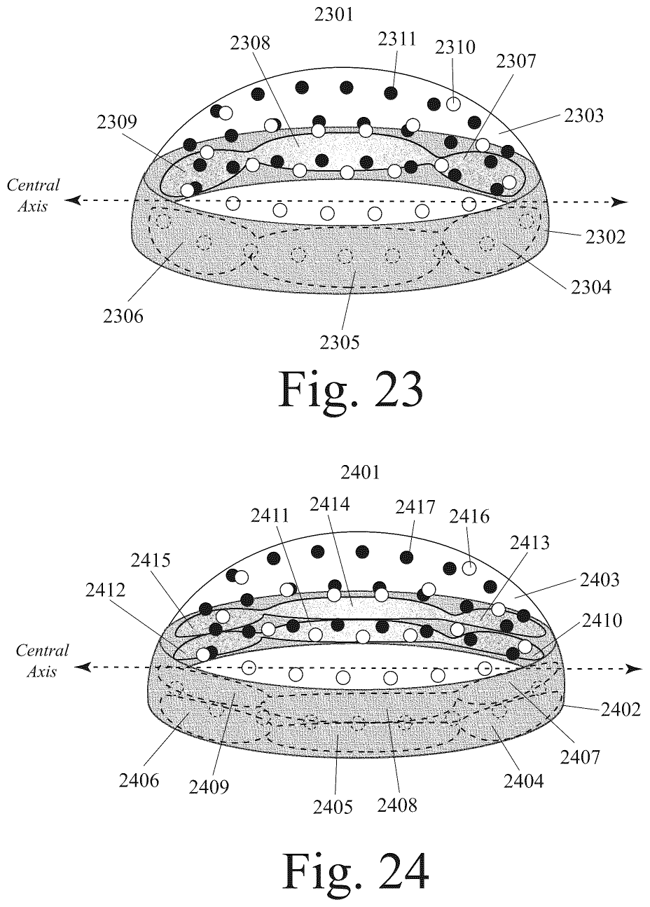

FIG. 23 shows a semi-transparent lateral (e.g. left side or right side) view of a cup which enables optical detection of abnormal breast tissue (e.g. breast cancer). In this example, there is an arcuate sequence and/or series of expandable chambers on each side of a virtual plane. In this example, the virtual plane passes through a central axis of the cup.

The bra cup shown in FIG. 23 comprises: an overall cup 2301 which is configured to be worn on a person's breast; a proximal portion 2302 of the cup; a distal portion 2303 of the cup, wherein the distal portion is farther from the chest wall than the proximal portion (when the cup is worn on a person's breast), and wherein the distal portion is more flexible or elastic than the proximal portion; a first sequence and/or series of expandable chambers (2304, 2305, and 2306) on a first side of a virtual plane which passes through the cup; a second sequence and/or series of expandable chambers (2307, 2308, and 2309) on a second side of the virtual plane; an array of light emitters (including light emitter 2310), wherein at least some of the light emitters are configured to be between the first sequence and/or series of expandable chambers and the breast; and an array of light receivers (including light receiver 2311), wherein at least some of the light receivers are configured to be between the second sequence and/or series of expandable chambers and the breast; wherein changes in light emitted from the light emitters and received by the light receivers which are caused by transmission of the light through the breast are analyzed to detect abnormal breast tissue and/or identify attributes of the abnormal breast tissue. Relevant variations discussed elsewhere in this disclosure or in priority-linked disclosures can also be applied to this example.

FIG. 24 shows a semi-transparent lateral (e.g. left side or right side) view of a cup which enables optical detection of abnormal breast tissue (e.g. breast cancer). In this example, there are two (parallel) series and/or sequences of expandable chambers on each side of a virtual plane. In this example, the virtual plane passes through a central axis of the cup.

The bra cup shown in FIG. 24 comprises: an overall cup 2401 which is configured to be worn on a person's breast; a proximal portion 2402 of the cup; a distal portion 2403 of the cup, wherein the distal portion is farther from the chest wall than the proximal portion (when the cup is worn on a person's breast), and wherein the distal portion is more flexible or elastic than the proximal portion; a first sequence and/or series of expandable chambers (2404, 2405, and 2406) on a first side of a virtual plane which passes through the cup; a second sequence and/or series of expandable chambers (2407, 2408, and 2409) on the first side of the virtual plane, wherein the second sequence and/or series is substantially parallel to the first set; a third sequence and/or series expandable chambers (2410, 2411, and 2412) on a second side of the virtual plane; a fourth sequence and/or series expandable chambers (2413, 2414, and 2415) on the second side of the virtual plane, wherein the fourth sequence and/or series is substantially parallel to the third set; an array of light emitters (including light emitter 2416), wherein at least some of the light emitters are configured to be between the first sequence and/or series of expandable chambers and the breast; and an array of light receivers (including light receiver 2417), wherein at least some of the light receivers are configured to be between the second sequence and/or series of expandable chambers and the breast; wherein changes in light emitted from the light emitters and received by the light receivers which are caused by transmission of the light through the breast are analyzed to detect abnormal breast tissue and/or identify attributes of the abnormal breast tissue. Relevant variations discussed elsewhere in this disclosure or in priority-linked disclosures can also be applied to this example.

I claim:

1. A bra for optical detection of abnormal breast tissue comprising:
a cup which is configured to be worn on a person's breast;
wherein the cup includes a proximal portion;
wherein the cup includes a distal portion, wherein the proximal portion is configured to be closer to the person's chest wall than the distal portion the cup is worn on the person's breast, and wherein the distal portion is more flexible or elastic than the proximal portion;
a first set of one or more expandable chambers on a first side of a virtual plane which passes through the cup;
a second set of one or more expandable chambers on a second side of the virtual plane;
an array of light emitters, wherein at least some of the light emitters are configured to be between the first set of one or more expandable chambers and the breast; and
an array of light receivers, wherein at least some of the light receivers are configured to be between the second set of one or more expandable chambers and the breast;
wherein changes in light emitted from the light emitters and received by the light receivers which are caused by interaction between the light and the breast are analyzed to detect abnormal breast tissue.

2. The bra in claim 1 wherein expandable chambers are expanded by being filled with a flowable substance.

3. The bra in claim 2 wherein the flowable substance is a gas.

4. The bra in claim 2 wherein the flowable substance is a liquid.

5. The bra in claim 1 wherein the virtual plane is vertical when the person is standing.

6. The bra in claim 1 wherein the virtual plane is oblique, spanning from an upper outer quadrant of the breast to a lower inner quadrant of a breast.

7. The bra in claim 1 wherein the proximal portion has a ring, band, column-section, or frustal shape.

8. The bra in claim 1 wherein a proximal perimeter of the proximal portion has a circular, elliptical, oval, teardrop, or crescent shape.

9. The bra in claim 1 wherein an expandable chamber between two other expandable chambers on a side of a virtual plane is larger and/or expanded more than the two other expandable chambers.

10. The bra in claim 1 wherein a cup further comprises an inner component located between light emitters and expandable chambers.

11. The bra in claim 1 wherein a cup further comprises an inner component located between light receivers and expandable chambers.

12. The bra in claim 1 wherein each expandable chamber is connected to a separate tube.

13. The bra in claim 1 wherein an expandable chamber has undulating, variable-width sections along its length.

14. The bra in claim 1 wherein light from light emitters is configured to be transmitted through the breast to the light receivers.

15. The bra in claim 1 wherein light from light emitters is configured to be reflected from the breast to the light receivers.
